(19) United States
(12) Patent Application Publication
(10) Pub. No.: US 2009/0170791 A1
(43) Pub. Date: Jul. 2, 2009

(54) MACROLONES

(75) Inventors: Sulejman Alihodzic, Zagreb (HR); John Michael Berge, Essex (GB); Catherine Simone Victoire Frydrych, Essex (GB); Samra Kapic, Zagreb (HR); Ivana Palej, Zagreb (HR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Smith Kline istrazivacki centar Zagreb d.o.o a corporation

(21) Appl. No.: 12/093,150

(22) PCT Filed: Nov. 7, 2006
(86) PCT No.: PCT/EP2006/010733

§ 371 Date: May 9, 2008

(30) Foreign Application Priority Data
Nov. 9, 2005 (EP) PCT EP2005 012038
May 11, 2006 (GB) 0609373.6

(51) Int. Cl. A61K 31/7042; C07H 17/08; A61P 31/04

(52) U.S. Cl. 514/28; 536/7.1

(57) ABSTRACT

A compound of formula (I)

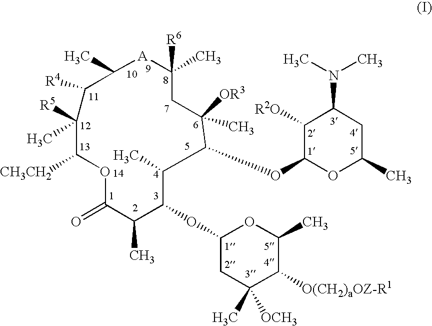

or a pharmaceutically acceptable derivative thereof, having antimicrobial activity, processes for their preparation, compositions containing them and to their use in medicine.

The present invention relates to novel semi-synthetic macrolides having antimicrobial activity, in particular antibacterial activity. More particularly, the invention relates to 15-membered macrolides substituted at the 4″ position, processes for their preparation, compositions containing them and to their use in medicine.

Macrolide antibacterial agents are known to be useful in the treatment or prevention of bacterial infections. However, the emergence of macrolide-resistant bacterial strains has resulted in the need to develop new macrolide compounds. For example, EP 0 895 999, WO 03/042228, WO 04/101585, WO 04/101586, WO 04/101587, WO 04/101588, WO 04/101589, WO 04/101590, WO 04/039822, WO 05/108412, and WO 05/108413 describe derivatives modified at the 4″ position of the macrolide ring having antibacterial activity.

According to the present invention, we have now found novel 15-membered macrolides substituted at the 4″ position which also have antimicrobial activity.

Thus, the present invention provides compounds of general formula (I)

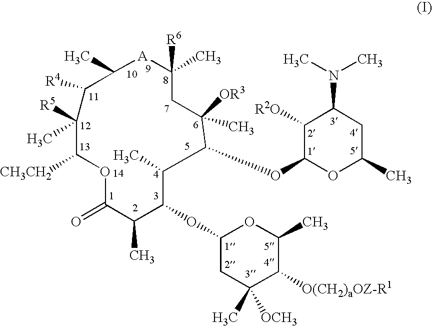

wherein:
Z represents C₃₋₆alkylene, C₃₋₆alkenylene or C₃₋₆alkynylene;
R¹ is a moiety:

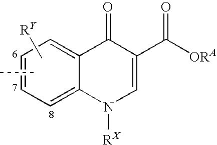

wherein
R^{A} is hydrogen, or C₁₋₆alkyl;
R^{X} represents —NR^{B}R^{C}, C₃₋₆cycloalkyl, C₁₋₆alkyl, C₃₋₆cycloalkylC₁₋₃alkyl, C₁₋₆alkoxy, —(CH₂)ₙC₁₋₄alkoxy or —(CH₂)ₙNR^{V}R^{W};
R^{Y} represents hydrogen or halogen;
R^{V} and R^{W} independently represent hydrogen or C₁₋₆alkyl;
R^{B} and R^{C} are each independently hydrogen, C₁₋₄alkyl or C₃₋₇cycloalkyl;
   or a moiety

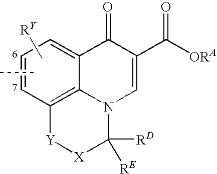

wherein:
R^{A} is hydrogen, or C₁₋₆alkyl;
R^{Y} represents hydrogen or halogen;
R^{D} and R^{E} independently represent hydrogen, or C₁₋₆alkyl such as methyl; and
Y and X independently represent O or CH₂,
with the proviso that when Y represents O, X represents CH₂ and when X represents O, Y represents CH₂;
   R² is hydrogen or a hydroxyl protecting group;
   R³ is hydrogen;
   R⁴ is hydroxy, C₃₋₆alkenyloxy, or C₁₋₆alkoxy,
   R⁵ is hydroxy, or
   R⁴ and R⁵ taken together with the intervening atoms form a cyclic group:

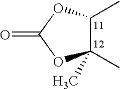

R⁶ is hydrogen;
A is a bivalent —N(R⁷)—CH₂— or —CH₂N(R⁷)—;
R⁷ is hydrogen or C₁₋₆alkyl;
a is an integer from 2 to 5
n is an integer from 2 to 4;
wherein the moiety R¹ is linked to the remainder of the molecule for the bicyclic moiety in the 6, 7 or 8 position or for the tricyclic moiety in the 6 or 7 position
and pharmaceutically acceptable derivatives thereof.

The term “pharmaceutically acceptable” as used herein means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Examples of pharmaceutically acceptable derivatives are salts, solvates, esters, carbamates and phosphate esters. Additional examples of pharmaceutically acceptable derivatives are salts, solvates and esters. Further examples of pharmaceutically acceptable derivatives are salts and esters, such as salts.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutically acceptable salt may be readily prepared by using a desired acid or base as appropriate. The salt may precipitate from solution and be collected by filtration or may be recovered by evaporation of the solvent. For example, an aqueous solution of an acid such as lactobionic acid may be added to a solution of a compound of formula (I) in a solvent such as acetonitrile, acetone or THF, and the resulting mixture evaporated to dryness, redissolved in water and lyophilised to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The skilled person will appreciate that where the compound of formula (I) contains more than one basic group bis salts (2:1 acid:compound of formula (I)) or tris salts (3:1 acid:compound of formula (I)) may also be formed and are salts according to the present invention.

Suitable addition salts are formed from inorganic or organic acids which form non-toxic salts and examples are lactobionate, mandelate (including (S)-(+)-mandelate, (R)-(−)-mandelate and (R,S)-mandelate), hydrochloride, hydrobromide, hydroiodide, sulfate, bisulfate, nitrate, phosphate, hydrogen phosphate, acetate, trifluoroacetate, maleate, malate, fumarate, lactate, tartrate, citrate, formate, gluconate, succinate, ethyl succinate (4-ethoxy-4-oxo-butanoate), pyruvate, oxalate, oxaloacetate, saccharate, benzoate, alkyl or aryl sulphonates (eg methanesulphonate, ethanesulphonate, benzenesulphonate or p-toluenesulphonate) and isethionate. In one embodiment, suitable salts include lactobionate, citrate, succinate, (L)-(+)-tartrate, (S)-(+)-mandalete and bis-(S)-(+)-mandalete, for example lactobionate, citrate, succinate and (L)-(+)-tartrate, such as lactobionate and citrate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Compounds of the invention may have both a basic and an acidic centre and may therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of formula (I) may form solvates (e.g. hydrates) and the invention also includes all such solvates.

The term “prodrug” as used herein means a compound which is converted within the body, e.g. by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series; Edward B. Roche, ed., “Bioreversible Carriers in Drug Design”, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of formula (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of formula (I) and their pharmaceutically acceptable derivatives.

With regard to stereoisomers, the compounds of formula (I) have more than one asymmetric carbon atom. In the general formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of structure (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or HPLC. A stereoisomeric mixture of the agent may also be prepared from a corresponding optically pure intermediate or by resolution, such as by HPLC, of the corresponding mixture using a suitable chiral support or by fractional crystallisation of the diastereoisomeric salts formed by reaction of the corresponding mixture with a suitable optically active acid or base, as appropriate.

The compounds of formula (I) may be in crystalline or amorphous form. Furthermore, some of the crystalline forms of the compounds of structure (I) may exist as polymorphs, which are included in the present invention.

In one aspect of the invention “a” represents 2 or 3.

In one aspect of the invention “a” represents 4 or 5.

In one aspect of the invention Z represents C₃₋₆alkylene such as n-propylene, n-butylene, or n-pentylene. In a further aspect, Z represents n-propylene or n-butylene. In another aspect, Z represents propylene, such as n-propylene.

In one aspect of the invention Z represents C₃₋₆alkenylene such as —CH₂CH═CH—, —CH═CHCH₂—, —CH═C(CH₃)— or C₃₋₆alkynylene such as —CH₂C≡C—, —CH₂CH₂C≡C—, —CH₂CH₂CH₂C≡C—.

In one aspect of the invention Z represents C₃₋₆alkenylene such as —CH₂CH═CH— or C₃₋₆alkynylene such as —CH₂C≡C—.

In one aspect of the invention ZR¹ represents —CH₂CH═CHR¹ or —CH₂C≡CR¹.

In one aspect of the invention R^{A} represents H.

In one aspect of the invention R^{A} represents ethyl.

In one aspect of the invention R^{B} represents C₁₋₆alkyl, such as methyl.

In one aspect of the invention R^{C} represents C₁₋₆alkyl, such as methyl.

In one aspect of the invention R^{D} represents methyl or hydrogen.

In one aspect of the invention R^{E} represents methyl or hydrogen.

In one aspect of the invention R^{X} represents represents —NR^{B}R^{C}, C₃₋₆cycloalkyl, C₁₋₆alkyl, C₁₋₆alkoxy, —(CH₂)ₙC₁₋₄alkoxy or —(CH₂)ₙNR^{V}R^{W}.

In one aspect of the invention R^{X} represents —NR^{B}R^{C}, such as —N(CH₃)₂.

In one aspect of the invention R^{X} represents C₁₋₆alkyl such as methyl, ethyl, n-propyl, i-propyl, n-butyl or t-butyl.

In a further aspect of the invention Rx represents C₁₋₆alkyl such as ethyl, i-propyl or t-butyl.

In one aspect of the invention R^{X} represents C₃₋₆ cycloalkyl such as cyclopropyl.

In one aspect of the invention R^{X} represents C₃₋₆cycloalkylC₁₋₃alkyl such as cyclopropylmethyl.

In one aspect of the invention R^{X} represents C₁₋₆alkoxy such as methoxy.

In one aspect of the invention R^{X} represents —CH₂CH₂C₁₋₆alkoxy such as —CH₂CH₂OCH₃.

In one aspect of the invention R^{X} represents —(CH₂)ₙNR^{V}R^{W} such as —CH₂CH₂N(CH₃)₂.

In one aspect of the invention R^{Y} may represent hydrogen, fluoro or chloro. In a further aspect, R^{Y} represents hydrogen.

In one aspect of the invention R^{Y} is located in the 5, 7 or 8 position on the quinolone moiety, such as the 7 position.

In one aspect of the invention A represents —N(R⁷)—CH₂—.

In one aspect of the invention X represents CH₂.

In one aspect of the invention Y represents CH₂.

In one aspect of the invention Y represents O.

In one aspect of the invention R¹ is a quinolone derived moiety.

In one aspect of the invention the quinolone moiety is linked to the remainder of the molecule through the 6 or 7 position.

In one aspect of the invention the quinolone moiety is linked as shown below:

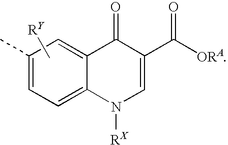

In one aspect of the invention the tricyclic moiety is linked as shown below:

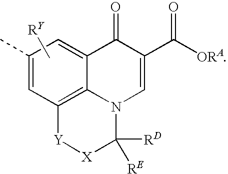

In one aspect of the invention the quinolone moiety is linked as shown below:

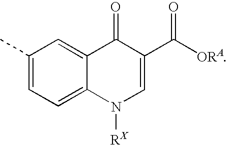

In one aspect of the invention the tricyclic moiety is linked as shown below:

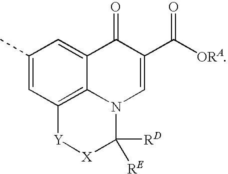

Compounds wherein R² represents a hydroxyl protecting group are in general intermediates for the preparation of other compounds of formula (I).

When the group OR² is a protected hydroxyl group this is conveniently an ether or an acyloxy group. Examples of particularly suitable ether groups include those in which R² is a trialkylsilyl (i.e. trimethylsilyl). When the group OR² represents an acyloxy group, then examples of suitable groups R² include acetyl or benzoyl.

In one aspect of the invention R² represents H.

In one aspect of the invention R⁴ represents hydroxyl.

In one aspect of the invention R⁴ represents hydroxyl or C₁₋₆alkoxy such as methoxy.

In one aspect of the invention R⁵ represents hydroxyl.

In one aspect of the invention R⁷ represents methyl.

The term “alkyl” as used herein as a group or a part of a group refers to a straight or branched hydrocarbon chain containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, isobutyl, isopropyl, t-butyl, hexyl. In one aspect, “alkyl” refers to a C₁₋₄alkyl group, for example methyl, ethyl, n-propyl, isopropyl, n-butyl, isobutyl or t-butyl.

The term “cycloalkyl” as used herein as a group or a part of a group refers to a non-aromatic monocyclic hydrocarbon ring containing the specified number of carbon atoms. For example, C₃₋₇cycloalkyl means a non-aromatic monocyclic hydrocarbon ring containing at least 3, and at most 7, carbon atoms such as, for example, cyclopropyl, cyclobutyl, cyclopentyl, cyclohexyl or cycloheptyl.

The term “cycloalkylalkyl” as used herein refers to a cycloalkyl group, as defined above, attached to the molecule via an alkyl group, as defined above, for example cyclopropylmethyl.

The term “alkoxy” as used herein refers to a straight or branched chain alkoxy group containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. In one aspect, “alkoxy” refers to a C₁₋₄alkoxy group, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Similarly, the term “C₃₋₆alkenyl” means a straight or branched alkenyl containing at least 3, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl.

The term “alkynyl” as used herein as a group or a part of a group refers to a straight or branched hydrocarbon chain containing the specified number of carbon atoms and containing at least one triple bond. For example, the term “C₃₋₆alkynyl” means a straight or branched alkynyl containing at least 3, and at most 6, carbon atoms and containing at least one triple bond. Examples of “alkynyl” as used herein include, but are not limited to, propynyl, 1-butynyl, 2-butynyl, 1-pentynyl and 3-methyl-1-butynyl.

The term “alkylene” as used herein refers to a linear or branched saturated hydrocarbon linker group containing the specified number of carbon atoms. Examples of such groups include methylene, ethylene, propylene and the like.

The term “alkenylene” as used herein refers to a linear or branched hydrocarbon linker group containing the specified number of carbon atoms, and containing one or more carbon-carbon double bonds. Examples of alkenylene groups include ethenylene, propenylene and the like.

The term “alkynylene” as used herein refers to a linear or branched hydrocarbon linker group containing the specified number of carbon atoms, and containing one or more carbon-carbon triple bonds. Examples of alkynylene groups include ethynylene, propynylene and the like.

The term “halogen” refers to a fluorine, chlorine, bromine or iodine atom.

It is to be understood that the present invention covers all combinations of the embodiments and representative examples described hereinabove.

Compounds of the invention include:
4″-O-{3-[3-(3-Carboxy-1-dimethylamino-4-oxo-1,4-dihydro-quinolin-6-yl)-prop-2-en-1-yloxy]-propyl}-azithromycin;
4″-O-{3-[3-(3-Carboxy-1-dimethylamino-4-oxo-1,4-dihydro-quinolin-6-yl)-prop-2-ynyloxy]-propyl}-azithromycin;
4″-O-{3-[3-(3-Carboxy-1-dimethylamino-4-oxo-1,4-dihydro-quinolin-6-yl)-propoxy]-propyl}-azithromycin;
4″-O-{3-[3-(3-Carboxy-1-cyclopropyl-4-oxo-1,4-dihydro-quinolin-6-yl)-prop-2-en-1-yloxy]-propyl}-azithromycin;
4″-O-{3-[3-(3-Carboxy-1-ethyl-4-oxo-1,4-dihydro-quinolin-6-yl)-prop-2-en-1-yloxy]-propyl}-azithromycin;
4″-O-{3-[3-(3-Carboxy-1-methoxyeth-2-yl-4-oxo-1,4-dihydro-quinolin-6-yl)-prop-2-en-1-yloxy]-propyl}-azithromycin;
4″-O-{3-[3-(3-Carboxy-1-t-butyl-4-oxo-1,4-dihydro-quinolin-6-yl)-prop-2-en-1-yloxy]-propyl}-azithromycin;
4″-O-{3-[3-(3-Carboxy-1-i-propyl-4-oxo-1,4-dihydro-quinolin-6-yl)-prop-2-en-1-yloxy]-propyl}-azithromycin;
4″-O-{3-[3-(3-Carboxy-1-dimethylaminoeth-2-yl-4-oxo-1,4-dihydro-quinolin-6-yl)-prop-2-en-1-yloxy]-propyl}-azithromycin;
4″-O-{3-[3-(3-Carboxy-1-cyclopropyl-4-oxo-1,4-dihydro-quinolin-6-yl)-propoxy]-propyl}-azithromycin;
4″-O-{3-[3-(3-Carboxy-1-ethyl-4-oxo-1,4-dihydro-quinolin-6-yl)-propoxy]-propyl}-azithromycin;
4″-O-{3-[3-(3-Carboxy-1-methoxyeth-2-yl-4-oxo-1,4-dihydro-quinolin-6-yl)-propoxy]-propyl}-azithromycin;
4″-O-{3-[3-(3-Carboxy-1-t-butyl-4-oxo-1,4-dihydro-quinolin-6-yl)-propoxy]-propyl}-azithromycin;
4″-O-{3-[3-(3-Carboxy-1-i-propyl-4-oxo-1,4-dihydro-quinolin-6-yl)-propoxy]-propyl}-azithromycin;
4″-O-{3-[3-(3-Carboxy-1-dimethylaminoethyl-4-oxo-1,4-dihydro-quinolin-6-yl)-propoxy]-propyl}-azithromycin;
2′-O-Acetyl-4″-O-{2-[3-(3-carboxy-1,4-dihydro-1-dimethylamino-4-oxo-6-quinolinyl)prop-2-enyl]-oxyethyl}-azithromycin 11,12-carbonate ethyl ester;
2′-O-Acetyl-4″-O-{2-[3-(3-carboxy-1,4-dihydro-1-dimethylamino-4-oxo-6-quinolinyl)propyl]-oxyethyl}-azithromycin 11,12-carbonate ethyl ester;
4″-O-{2-[3-(3-Carboxy-1,4-dihydro-1-dimethylamino-4-oxo-6-quinolinyl)propyl]-oxyethyl}-azithromycin;
2′-O-Acetyl-4″-O-{2-[3-(3-carboxy-1,4-dihydro-1-ethyl-4-oxo-6-quinolinyl)prop-2-enyl]-oxyethyl}-azithromycin 11,12-carbonate ethyl ester;
2′-O-Acetyl-4″-O-{2-[3-(3-carboxy-1,4-dihydro-1-ethyl-4-oxo-6-quinolinyl)propyl]-oxyethyl}-azithromycin 11,12-carbonate ethyl ester;
4″-O-{2-[3-(3-Carboxy-1,4-dihydro-1-ethyl-4-oxo-6-quinolinyl)propyl]-oxyethyl}-azithromycin;
2′-O-Acetyl-4″-O-{5-[3-(3-carboxy-1-ethyl-4-oxo-1,4-dihydro-quinolin-6-yl)-prop-2-en-1-yloxy]-pentyl}-azithromycin-11,12-cyclic carbonate;
4″-O-{5-[3-(3-carboxy-1-ethyl-4-oxo-1,4-dihydro-quinolin-6-yl)-prop-2-en-1-yloxy]-pentyl}-azithromycin;
4″-O-{5-[3-(3-carboxy-1-ethyl-4-oxo-1,4-dihydro-quinolin-6-yl)-propyloxy]-pentyl}-azithromycin;
2′-O-Acetyl-4″-O-{2-[3-(6-carboxy-3,3-dimethyl-7-oxo-1H,7H-[1,3oxazino]5,4,3-ij]-9-quinolinyl)prop-2-enyl]-oxyethyl}-azithromycin 11,12-carbonate ethyl ester;
2′-O-Acetyl-4″-O-{2-[3-(6-carboxy-3,3-dimethyl-7-oxo-1H,7H-[1,3oxazino]5,4,3-ij]-9-quinolinyl)prop-1-enyl]-oxyethyl}-azithromycin 11,12-carbonate ethyl ester;
2′-O-Acetyl-4″-O-{2-[3-(6-carboxy-3,3-dimethyl-7-oxo-1H,7H-[1,3oxazino]5,4,3-ij]-9-quinolinyl)prop-1-yl]-oxyethyl}-azithromycin 11,12-carbonate ethyl ester;
4″-O-{2-[3-(6-carboxy-3,3-dimethyl-7-oxo-1H,7H-[1,3oxazino]5,4,3-ij]-9-quinolinyl)prop-1-yl]-oxyethyl}-azithromycin;
4″-O-{3-[4-(3-Carboxy-1-dimethylamino-4-oxo-1,4-dihydro-quinolin-6-yl)-but-3-ynyloxy]-propyl}-azithromycin;
4″-O-{3-[4-(3-Carboxy-1-ethyl-4-oxo-1,4-dihydro-quinolin-6-yl)-but-3-ynyloxy]-propyl}-azithromycin;
4″-O-{3-[5-(3-Carboxy-1-dimethylamino-4-oxo-1,4-dihydro-quinolin-6-yl)-pent-4-ynyloxy]-propyl}-azithromycin;
4″-O-{3-[5-(3-Carboxy-1-ethyl-4-oxo-1,4-dihydro-quinolin-6-yl)-pent-4-ynyloxy]-propyl}-azithromycin;
4″-O-{3-[4-(3-Carboxy-1-cyclopropyl-4-oxo-1,4-dihydro-quinolin-6-yl)-but-3-ynyloxy]-propyl}-azithromycin;
4″-O-{3-[4-(3-Carboxy-1-dimethylamino-4-oxo-1,4-dihydro-quinolin-6-yl)-butoxy]-propyl}-azithromycin;
4″-O-{3-[4-(3-Carboxy-1-ethyl-4-oxo-1,4-dihydro-quinolin-6-yl)-butoxy]-propyl}-azithromycin;
4″-O-{3-[5-(3-Carboxy-1-dimethylamino-4-oxo-1,4-dihydro-quinolin-6-yl)-pentoxy]-propyl}-azithromycin;
4″-O-{3-[5-(3-Carboxy-1-ethyl-4-oxo-1,4-dihydro-quinolin-6-yl)-pentoxy]-propyl}-azithromycin;
4″-O-{3-[4-(3-Carboxy-1-cyclopropyl-4-oxo-1,4-dihydro-quinolin-6-yl)-butoxy]-propyl}-azithromycin;
4″-O-{3-[3-(5-Carboxy-3,3-dimethyl-6-oxo-1H,6H-2-oxa-3a-aza-phenalen-8-yl)-prop-2-ynyloxy]-propyl}-azithromycin;
4″-O-{3-[3-(5-Carboxy-3,3-dimethyl-6-oxo-1H,6H-2-oxa-3a-aza-phenalen-8-yl)-propoxy]-propyl}-azithromycin;
4″-O-{3-[4-(5-Carboxy-3,3-dimethyl-6-oxo-1H,6H-2-oxa-3a-aza-phenalen-8-yl)-but-3-ynyloxy]-propyl}-azithromycin;
4″-O-{3-[4-(5-Carboxy-3,3-dimethyl-6-oxo-1H,6H-2-oxa-3a-aza-phenalen-8-yl)-butoxy]-propyl}-azithromycin;
2′-O-Acetyl-4″-O-{2-[3-(3-carboxy-1,4-dihydro-1-(2-methoxyethyl)-4-oxo-6-quinolinyl)prop-2-enyl]-oxyethyl}-azithromycin 11,12-carbonate;
2′-O-Acetyl-4″-O-{2-[3-(3-carboxy-1,4-dihydro-1-(2-methoxyethyl)-4-oxo-6-quinolinyl)prop-1-enyl]-oxyethyl}-azithromycin 11,12-carbonate;
2′-O-Acetyl-4″-O-{2-[3-(3-carboxy-1,4-dihydro-1-(2-methoxyethyl)-4-oxo-6-quinolinyl)prop-1-yl]-oxyethyl}-azithromycin 11,12-carbonate;
4″-O-{2-[3-(3-Carboxy-1,4-dihydro-1-(2-methoxyethyl)-4-oxo-6-quinolinyl)prop-1-yl]-oxyethyl}-azithromycin;
2′-O-Acetyl-4″-O-{2-[3-(3-carboxy-1,4-dihydro-1-methoxy-4-oxo-6-quinolinyl)prop-2-enyl]-oxyethyl}-azithromycin 11,12-carbonate;
2′-O-Acetyl-4″-O-{2-[3-(3-carboxy-1,4-dihydro-1-methoxy-4-oxo-6-quinolinyl)prop-1-yl]-oxyethyl}-azithromycin 11,12-carbonate;
4″-O-{2-[3-(3-Carboxy-1,4-dihydro-1-methoxy-4-oxo-6-quinolinyl)prop-1-yl]-oxyethyl}-azithromycin;
4″-O-{2-[3-(3-carboxy-1,4-dihydro-1-propyl-4-oxo-6-quinolinyl)prop-2-enyl]-oxyethyl}-azithromycin;
4″-O-{2-[3-(3-carboxy-1,4-dihydro-1-propyl-4-oxo-6-quinolinyl)propyl]-oxyethyl}-azithromycin;
4″-O-{2-[3-(3-carboxy-1,4-dihydro-1-methyl-4-oxo-6-quinolinyl)prop-2-enyl]-oxyethyl}-azithromycin;
4″-O-{2-[3-(3-carboxy-1,4-dihydro-1-methyl-4-oxo-6-quinolinyl)prop-1-enyl]-oxyethyl}-azithromycin;
4″-O-{2-[3-(3-Carboxy-1,4-dihydro-1-methyl-4-oxo-6-quinolinyl)propyl]-oxyethyl}-azithromycin;
4″-O-{2-[3-(3-Carboxy-1,4-dihydro-4-oxo-1-iso-propyl-6-quinolinyl)prop-2-enyl]-oxyethyl}-azithromycin;
4″-O-{2-[3-(3-Carboxy-1,4-dihydro-4-oxo-1-iso-propyl-6-quinolinyl)prop-1-enyl]-oxyethyl}-azithromycin;
4″-O-{2-[3-(3-Carboxy-1,4-dihydro-4-oxo-1-iso-propyl-6-quinolinyl)propyl]-oxyethyl}-azithromycin;
4″-O-{2-[3-(3-Carboxy-1,4-dihydro-4-oxo-1-cyclopropyl-6-quinolinyl)prop-2-enyl]-oxyethyl}-azithromycin;
4″-O-{2-[3-(3-Carboxy-1,4-dihydro-4-oxo-1-cyclopropyl-6-quinolinyl)prop-1-enyl]-oxyethyl}-azithromycin;
4″-O-{2-[3-(3-Carboxy-1,4-dihydro-1-cyclopropyl-4-oxo-6-quinolinyl)propyl]-oxyethyl}-azithromycin;
4″-O-{2-[3-(2-Carboxy-6,7-dihydro-1H,5H-pyrido[3,2,1-ij]-1-oxo-9-quinolinyl)prop-2-enyl]-oxyethyl}-azithromycin;
4″-O-{2-[3-(2-Carboxy-6,7-dihydro-1H,5H-pyrido[3,2,1-ij]-1-oxo-9-quinolinyl)prop-1-enyl]-oxyethyl}-azithromycin;
4″-O-{2-[3-(2-Carboxy-6,7-dihydro-1H,5H-pyrido[3,2,1-ij]-1-oxo-9-quinolinyl)propyl]-oxyethyl}-azithromycin;
4″-O-{2-[3-(6-Carboxy-7-oxo-2,3-dihydro-7H-[1,4]oxazino[2,3,4-ij]quinolin-9-yl) prop-2-enyl]-oxyethyl}-azithromycin;
4″-O-{2-[3-(6-Carboxy-7-oxo-2,3-dihydro-7H-[1,4]oxazino[2,3,4-ij]quinolin-9-yl) prop-1-enyl]-oxyethyl}-azithromycin;
4″-O-{2-[3-(6-Carboxy-7-oxo-2,3-dihydro-7H-[1,4]oxazino[2,3,4-ij]quinolin-9-yl) propyl]-oxyethyl}-azithromycin;
4″-O-{2-[3-(6-Carboxy-3,3-dimethyl-7-oxo-2,3-dihydro-7H-[1,4]oxazino[2,3,4-ij]quinolin-9-yl)prop-2-enyl]-oxyethyl}-azithromycin;
4″-O-{2-[3-(6-Carboxy-3,3-dimethyl-7-oxo-2,3-dihydro-7H-[1,4]oxazino[2,3,4-ij]quinolin-9-yl)prop-1-enyl]-oxyethyl}-azithromycin;
4″-O-{2-[3-(6-Carboxy-3,3-dimethyl-7-oxo-2,3-dihydro-7H-[1,4]oxazino[2,3,4-ij]quinolin-9-yl)propyl]-oxyethyl}-azithromycin;
4″-O-{2-[3-(3-Carboxy-1,4-dihydro-4-oxo-1-cyclo-propylmethyl-6-quinolinyl)prop-2-enyl]-oxyethyl}-azithromycin;
4″-O-{2-[3-(3-Carboxy-1,4-dihydro-4-oxo-1-cyclo-propylmethyl-6-quinolinyl)prop-1-enyl]-oxyethyl}-azithromycin;
4″-O-{2-[3-(3-Carboxy-1,4-dihydro-1-cyclo-propylmethyl-4-oxo-6-quinolinyl)propyl]-oxyethyl}-azithromycin;
4″-O-{2-[3-(3-carboxy-1,4-dihydro-1-butyl-4-oxo-6-quinolinyl)prop-2-enyl]-oxyethyl}-azithromycin ethyl ester;
4″-O-{2-[3-(3-carboxy-1,4-dihydro-1-butyl-4-oxo-6-quinolinyl)propyl]-oxyethyl}-azithromycin ethyl ester;
4″-O-{2-[3-(3-carboxy-1,4-dihydro-1-butyl-4-oxo-6-quinolinyl)propyl]-oxyethyl}-azithromycin;
2′-O-Acetyl-4″-O-{2-[3-(3-carboxy-1,4-dihydro-1-ethyl-4-oxo-6-quinolinyl)prop-1-enyl]-oxyethyl}-azithromycin 11,12-carbonate;
4″-O-{2-[3-(3-Carboxy-1,4-dihydro-1-(2-methoxyethyl)-4-oxo-6-quinolinyl)prop-1-enyl]-oxyethyl}-azithromycin;
2′-O-Acetyl-4″-O-{2-[2-(3-carboxy-1,4-dihydro-1-ethyl-4-oxo-6-quinolinyl)-1-propenyl]-oxyethyl}-azithromycin 11,12-carbonate ethyl ester;
4″-O-{2-[2-(3-carboxy-1,4-dihydro-1-ethyl-4-oxo-6-quinolinyl)-prop-1-enyl]-oxyethyl}-azithromycin;
4″-O-{3-[3-(3-Carboxy-1-ethyl-4-oxo-1,4-dihydro-quinolin-6-yl)-prop-2-ynyloxy]-propyl}-azithromycin;
4″-O-{3-[3-(3-Carboxy-1-cyclopropyl-4-oxo-1,4-dihydro-quinolin-6-yl)-prop-2-ynyloxy]-propyl}-azithromycin;
4″-O-{2-[3-(3-Carboxy-1,4-dihydro-1-ethyl-4-oxo-6-quinolinyl)butyl]-oxyethyl}-azithromycin;
4″-O-{2-[3-(3-Carboxy-1,4-dihydro-1-ethyl-4-oxo-7-quinolinyl)propyl]-oxyethyl}-azithromycin;
4″-O-{2-[3-(3-Carboxy-1,4-dihydro-1-cyclopropyl-6-fluoro-4-oxo-7-quinolinyl)propyl]-oxyethyl}-azithromycin;
   or a pharmaceutically acceptable derivative thereof.

Additionally compounds of the invention may include one or more compounds:
4″-O-{4-[3-(3-carboxy-1-ethyl-4-oxo-1,4-dihydro-quinolin-6-yl)-prop-2-en-1-yloxy]-butyl}-azithromycin;
4″-O-{4-[3-(3-carboxy-1-cyclopropyl-4-oxo-1,4-dihydro-quinolin-6-yl)-prop-2-en-1-yloxy]-butyl}-azithromycin;
4″-O-{4-[3-(3-carboxy-1-dimethylamino-4-oxo-1,4-dihydro-quinolin-6-yl)-prop-2-en-1-yloxy]-butyl}-azithromycin;
4″-O-{4-[3-(3-carboxy-1-methoxyeth-2-yl-4-oxo-1,4-dihydro-quinolin-6-yl)-prop-2-en-1-yloxy]-butyl}-azithromycin;
4″-O-{4-[3-(3-carboxy-1-ethyl-4-oxo-1,4-dihydro-quinolin-6-yl)-propyloxy]-butyl}-azithromycin;
4″-O-{4-[3-(3-carboxy-1-cyclopropyl-4-oxo-1,4-dihydro-quinolin-6-yl)-propyloxy]-butyl}-azithromycin;
4″-O-{4-[3-(3-carboxy-1-dimethylamino-4-oxo-1,4-dihydro-quinolin-6-yl)-propyloxy]-butyl}-azithromycin;
4″-O-{4-[3-(3-carboxy-1-methoxyeth-2-yl-4-oxo-1,4-dihydro-quinolin-6-yl)-propyloxy]-butyl}-azithromycin;
   or a pharmaceutically acceptable derivative thereof.

Compounds according to the invention may exhibit a broad spectrum of antimicrobial activity, in particular antibacterial activity, against a wide range of clinical pathogenic microorganisms. Using a standard microtiter broth serial dilution test, compounds of the invention have been found to exhibit useful levels of activity against one or more of a range of pathogenic microorganisims, for example gram positive bacteria. The compounds of the invention may be active against strains which include Staphylococcus aureus, Streptopococcus pneumoniae, Moraxella catarrhalis, Streptococcus pyogenes, Haemophilus influenzae, Enterococcus faecalis, Chlamydia pneumoniae, Mycoplasma pneumoniae and Legionella pneumophila, such as Staphylococcus aureus, Streptopococcus pneumoniae, Enterococcus faecalis and Streptococcus pyogenes. The compounds of the invention may also be active against resistant strains, for example erythromycin resistant strains. Thus, for example, the compounds of the invention may be active against erythromycin resistant strains of Streptococcus pneumoniae, Streptococcus pyogenes and Staphylococcus aureus.

One or more compounds of the invention may show higher activity against Haemophilus influenzae, than corresponding compounds based on a clarithromycin template. Furthermore, one or more compounds of the invention may show better protein binding properties in humans and/or more preferable P450 interaction profiles than known compounds in this general class.

The compounds of the invention may therefore be useful for treating a variety of diseases caused by pathogenic microorganisms, in particular bacteria, in human beings and animals. It will be appreciated that reference to treatment includes acute treatment or prophylaxis as well as the alleviation of established symptoms.

One or more compounds of the invention may also be more efficacious, show greater selectivity, have fewer side effects, have a longer duration of action, be more bioavailable by the preferred route, have more suitable pharmacodynamic or pharmacokinetic properties, or have other more desirable properties, for example better physical properties such as crystallinity than similar known compounds.

Thus, according to another aspect of the present invention we provide a compound of formula (I) or a pharmaceutically acceptable derivative thereof for use in therapy.

According to a further aspect of the invention we provide a compound of formula (I) or a pharmaceutically acceptable derivative thereof for use in the treatment or prophylaxis of systemic or topical microbial infections in a human or animal body.

According to a further aspect of the invention we provide the use of a compound of formula (I) or a pharmaceutically acceptable derivative thereof in the manufacture of a medicament for use in the treatment or prophylaxis of systemic or topical microbial infections in a human or animal body.

According to a yet further aspect of the invention we provide a method of treatment of the human or non-human animal body to combat microbial infections comprising administration to a body in need of such treatment of an effective amount of a compound of formula (I) or a pharmaceutically acceptable derivative thereof.

Compounds of the invention may also have use in the treatment of conjunctiva and conjunctivitis.

While it is possible that, for use in therapy, a compound of the invention may be administered as the raw chemical it is preferable to present the active ingredient as a pharmaceutical formulation eg when the agent is in admixture with a suitable pharmaceutical excipient, diluent or carrier selected with regard to the intended route of administration and standard pharmaceutical practice.

Accordingly, in one aspect, the present invention provides a pharmaceutical composition or formulation comprising a compound of the invention or a pharmaceutically acceptable derivative thereof in association with a pharmaceutically acceptable excipient, diluent and/or carrier. The excipient, diluent and/or carrier must be “acceptable” in the sense of being compatible with the other ingredients of the formulation and not deleterious to the recipient thereof.

In another aspect, the invention provides a pharmaceutical composition comprising, as active ingredient, a compound of the invention or a pharmaceutically acceptable derivative thereof in association with a pharmaceutically acceptable excipient, diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention and a pharmaceutically acceptable excipient, diluent and/or carrier (including combinations thereof).

There is further provided by the present invention a process of preparing a pharmaceutical composition, which process comprises mixing a compound of the invention or a pharmaceutically acceptable derivative thereof, together with a pharmaceutically acceptable excipient, diluent and/or carrier.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, diluents and/or carriers. Acceptable excipients, diluents and carriers for therapetic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

It is to be understood that not all of the compounds need be administered by the same route. Likewise, if the composition comprises more than one active component, then those components may be administered by different routes.

The compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually). In one aspect of the invention, the agents are delivered orally, hence, the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent, and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (e.g. by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, e.g. sterile, pyrogen-free water, before use.

The compounds of the invention can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, calcium sulphate, dibasic calcium phosphate and glycine, manitol, pregelatinised starch, corn starch, potato starch, disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin or HPMC (hydroxypropyl methylcellulose) capsules. Preferred excipients in this regard include microcrystalline cellulose, lactose, calcium carbonate, calcium sulphate, dibasic calcium phosphate and, manitol, pregelatinised starch, corn starch, potato starch or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Capsules, may be filled with a powder (of medicament alone or as blend with selected filler(s)) or alternatively a liquid, each comprising one or more compounds of formula (I) and a carrier. Where the capsule is filled with a powder the compounds of formula (I) and/or the carrier may be milled or micronised to provide material with an appropriate particle size.

Compounds of the invention may be susceptible to acid degradation after ingestion and may therefore require a coating such as an enteric coating when administered orally as a tablet or capsule.

The tablet or capsule, as appropriate, may, for example be coated by a thin film such as a EUDRAGIT® film available from Röhm Pharma Polymers, which allows controlled dissolution in the gastrointestinal tract. The films are available as cationic polymers such as EUDRAGIT® E 100 (aminoalkyl methacylate copolymers) or as anionic acrylic polymers such as EUDRAGIT® L (methacrylic acid copolymers) and EUDRAGIT S. Permeable acrylic polymers such as EUDRAGIT® RL (ammino methacrylate copolymer) and EUDRAGIT® RS are also available.

These coating formulations may be prepared as an aqueous dispersion including optional ingredients such as talc, silicone antifoam emulsion, polyethylene glycol. Alternatively the coating formulation may be prepared as an organic polymer solution.

Alternatively, tablets may be coated using OPADRY® (Surelease®) coating systems, available from Colorcon. Aqueous systems generally comprise up to 15% w/w of OPADRY®. Organic solvent systems generally comprise up to 5% w/w of OPADRY®.

The coatings may be prepared by known techniques, for example by;
1. weighing the required quantity of OPADRY® film coating system,
2. weighing the required quantity of water or other solvent(s) into a mixing vessel,
3. with a mixing propeller in the centre of the vessel and as close to the bottom of the vessel as possible, stirring the solvents to form a vortex without drawing air into the liquid,
4. steadily and quickly adding the OPADRY® powder to the vortex, avoiding powder flotation on the liquid surface,
5. increasing the stirrer speed in order to maintain the vortex, if required, and
6. after all the powder ingredients have been added, reducing the mixer speed and continuing mixing for approximately 45 minutes.

Coatings can be applied by known techniques, using tablet coating machines.

The thickness of the coating applied is generally in the range 5 to 35 microns such as 10 to 30 microns, more specifically 10 or 20 microns, depending on the required effect.

Alternatively, the tablet or a capsule, as appropriate, may be filled into another capsule (preferably a HPMC capsule such as Capsugel®) to provide either a tablet in capsule or capsule in capsule configuration, which when administered to a patient yields controlled dissolution in the gastrointestinal tract thereby providing a similar effect to an enteric coating.

Thus in one aspect the invention provides a solid dose formulation of a compound of formula (I) wherein said formulation has an enteric coating.

In another aspect the invention provides a solid dose formulation comprising a protective capsules as outer layer, for example as a tablet in a capsule or a capsule in a capsule.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories e.g. containing conventional suppository bases for use in human or veterinary medicine or as pessaries e.g. containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g. eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be dermally or transdermally administered, for example, by use of a skin patch.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebuliser.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may for example be used for topical administration with other active ingredients such as corticosteroids or antifungals as appropriate.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compositions may contain from 0.01-99% of the active material. For topical administration, for example, the composition will generally contain from 0.01-10%, more preferably 0.01-1% of the active material.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

For systemic administration the daily dose as employed for adult human treatment it will range from 2-100 mg/kg body weight, preferably 5-60 mg/kg body weight, which may be administered in 1 to 4 daily doses, for example, depending on the route of administration and the condition of the patient. When the composition comprises dosage units, each unit will preferably contain 200 mg to 1 g of active ingredient. The duration of treatment will be dictated by the rate of response rather than by arbitrary numbers of days.

Compounds of general formula (I) and pharmaceutically acceptable derivatives thereof may be prepared by the general methods outlined hereinafter, said methods constituting a further aspect of the invention. In the following description, the groups generally have the meaning defined for the compounds of formula (I) unless otherwise stated.

A comprehensive discussion of the ways in which sensitive groups may be protected and methods for cleaving the resulting protected derivatives is given by for example T. W. Greene and P. G. M Wuts in Protective Groups in Organic Synthesis 2^{nd} ed., John Wiley & Son, Inc 1991 and by P. J. Kocienski in Protecting Groups, Georg Thieme Verlag 1994 which are incorporated herein by reference. Examples of suitable amino protecting groups include acyl type protecting groups (e.g. formyl, trifluoroacetyl and acetyl), aromatic urethane type protecting groups (e.g. benzyloxycarbonyl (Cbz) and substituted Cbz, and 9-fluorenylmethoxycarbonyl (Fmoc)), aliphatic urethane protecting groups (e.g. t-butyloxycarbonyl (Boc), isopropyloxycarbonyl and cyclohexyloxycarbonyl) and alkyl type protecting groups (e.g. benzyl, trityl and chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate. Hydroxy groups may be protected by reaction of for example acetic anhydride, benzoic anhydride or a trialkylsilyl chloride in an aprotic solvent. Examples of aprotic solvents are dichloromethane, N,N-dimethylformamide, dimethylsulfoxide, tetrahydrofuran and the like.

The compounds of general formula (I) and derivatives thereof may be purified by conventional methods known in the art. For example, the compounds may be purified by HPLC using an aqueous solution of an acid such as formic acid or trifluoroacetic acid with an organic co-solvent such as acetonitrile or methanol. Alternatively, said compounds may be purified by crystallisation, chromatograph and/or differential partition between aqueous and organic solvents.

Compounds of formula (I) wherein Z represents C₃₋₆alkylene may be prepared by the general method below, which comprises:
a) hydrogenation of a compound of formula (Ia)

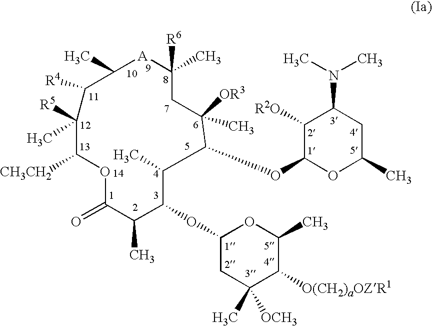

or a protected derivative thereof wherein:
R¹, R², R³, R⁴, R⁵, R⁶, a, and A, are as defined above for compounds of formula (I), or R⁴ and R⁵, taken together with the intervening atoms form a cyclic group:

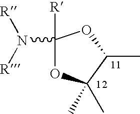

in which R′ represents hydrogen or methyl; R″ and R′″ independently represent C₁₋₃alkyl,
and
Z′ represents C₃₋₆alkenylene or C₃₋₆alkynylene;
b) optionally followed by deprotection, if necessary, to provide a compound of formula (I).

The hydrogenation step (step a) may, for example, be performed at greater than atmospheric pressue over a period of 3 to 20 hours, such as:
from about 7 hours to about 15 hours at about 3 to 5 bars for compounds of formula (Ia) where Z′ is C₃₋₆alkenylene, and
about 15 hours at about 2 bars such as 2.2 bars for compounds of formula (Ia) where Z′ is C₃₋₆alkynylene.

Compounds of formula (I) wherein a=2 and Z is n-propylene can be prepared according to the scheme A below via reaction of intermediates of the type of compounds of formula (IV), a=2, with intermediates of the type of compounds formula (II) or (III) described below.

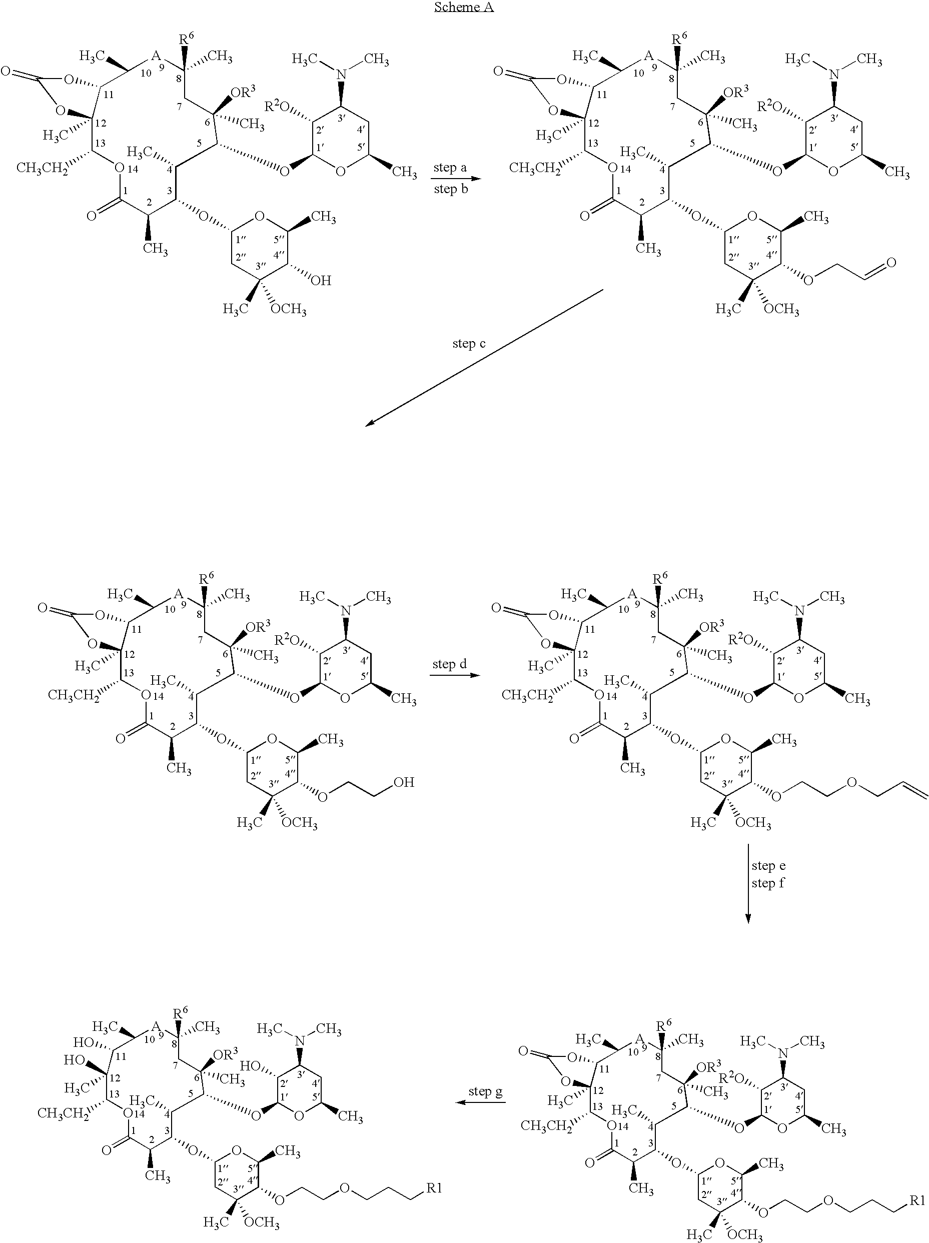

Step a involves the alkylation of the 4″-hydroxy group of, for example, 2′-acetyl-11,12-carbonate azithromycin with allyl t-butyl carbonate under palladium catalysed conditions, typically using excess allyl t-butyl carbonate in THF and tetrakis(triphenylphosphino)palladium (0) at reflux for 24-48 h.

Step b involves the ozonolysis of the product formed in step a, typically using ozone at −78° C. in DCM/MeOH protecting the amine functions from oxidation by low temperature salt formation with TFA. Cleavage of the intermediate ozonide is achieved, for example, with a mixture of triethylamine and dimethyl sulfide at −78° C. to room temperature for 0.5-1 h.

Step c involves the reduction of the product from step b, typically with sodium borohydride in MeOH at room temperature for 3 h.

Step d involves O-allylation of the product formed in step c with allyl t-butyl carbonate under palladium catalysed conditions, typically using excess allyl t-butyl carbonate in THF and tetrakis(triphenylphosphino)palladium (0) at reflux for 2-48 h.

Step e involves the reaction of the product formed in step d with an intermediate of type (II) or (III), wherein L represent iodo or bromo, under palladium catalysed conditions, typically palladium (II) diacetate in acetonitrile and triethylamine.

Step f involves the reduction of the products formed in step e, typically using 10% palladium on charcoal, hydrogen at atmospheric pressure and room temperature in DCM.

Step g involves the removal of the 11,12 and 2′-protecting groups, typically using 10% aqueous potassium carbonate solution in acetonitrile at 80° C. for 6-24 h.

It will be clear to persons skilled in the art that corresponding compounds of formula (I), wherein Z represents C₃alkenylene may be prepared by the methodology of scheme A above but omitting step f.

Compounds of formula (I), where a=3 and Z is n-propylene may be prepared by the route shown below in scheme B.

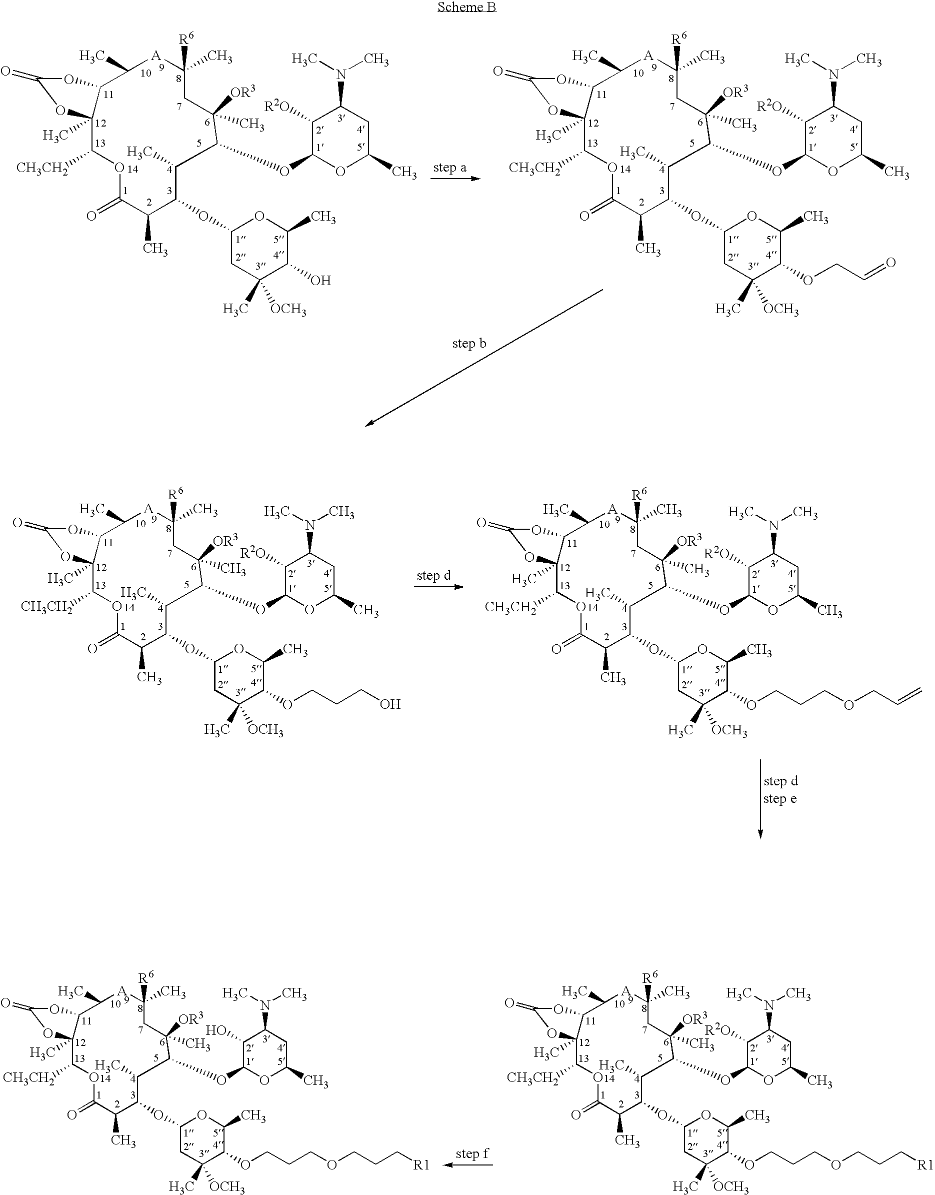

Step a involves the alkylation of the 4″-hydroxy group of, for example, 2′-acetyl-11,12-carbonate azithromycin with allyl t-butyl carbonate under palladium catalysed conditions, typically using excess allyl t-butyl carbonate in THF and tetrakis(triphenylphosphino)palladium (0) at reflux for 24-48 h.

Step b involves the hydroboration of the product formed in step a followed by oxidative cleavage of the carbon-boron bond to form an alcohol, typically reaction with 1M borane in THF at room temperature for 24 h followed by treatment with 27% hydrogen peroxide in 2M sodium hydroxide at room temperature for 0.5 h.

Step c involves O-allylation of the product formed in step b, which may be effected, with allyl t-butyl carbonate under palladium catalysed conditions, typically using excess allyl t-butyl carbonate in THF and tetrakis(triphenylphosphino)palladium (0) at reflux for 2-48 h.

Step d involves the reaction of the product formed in step c with an Intermediate of type (II) or (III), wherein L is iodo or bromo, under palladium catalysed conditions, such as palladium (II) diacetate in acetonitrle and triethylamine.

Step e involves the reduction of the products formed in step d, for example, using 10% palladium on charcoal, hydrogen at atmospheric pressure and room temperature in DCM.

Step f involves the removal of the 11,12 and 2′-protecting groups, for example, using 10% aqueous potassium carbonate solution in acetonitrile at 80° C. for 6-24 h.

Compounds of formula (I) wherein Z represents C₃₋₆alkenylene or C₃₋₆alkynylene, may be prepared by reacting a compound of formula (II) or (III)

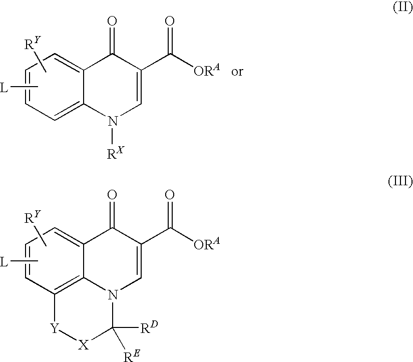

or a protected derivative thereof wherein:
R^{A}, R^{D}, R^{E}, R^{X} and R^{Y} are as defined above for compounds of formula (I), and
L represents a leaving group, for example halogen such as iodo or bromo, with a compound of formula (IV):

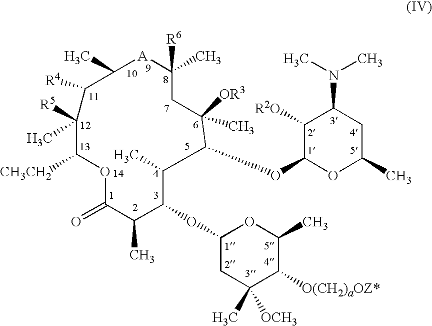

or a protected derivative thereof wherein:
R², R³, R⁴, R⁵, R⁶, A and a are as defined for compounds of formula (Ia),
and Z* represents C₃₋₆alkenyl or C₃₋₆alkynyl.

Generally the unsaturated bond in the moiety Z* is terminal, in that, it is in the part of the molecule away from the oxygen linker and able to “react” with the compounds of formula (II) or (III), as appropriate.

The reaction may be effected under a suitably inert atmosphere in the presence of an appropriate catalyst, generally under basic conditions, at a slightly elevated temperature.

Where Z* is C₃₋₆alkenyl the reaction may be effected by treating a compound of formula (IV) with a compound of formula (II) or (III) in a suitable solvent, for example, DMF or DMA under an inert atmosphere, such as argon, in the presence of a palladium catalyst, such as palladium acetate (Pd(OAc)₂) or trans-di-μ-acetatobis(2-(di-o-tolyl triphenylphosphine (TOTF), in the presence of a trialkylamine base, such as triethylamine, at elevated temperatures in the range 75° C.-125° C. After filtration and work-up the relevant product is obtained.

When Z* is C₃₋₆alkynyl the reaction may be effected by activating a compound of formula (II) or (III) in a suitable solvent, for example acetonitrile, in the presence of a catalyst such as copper iodide and an organic base such as triethylamine, under an inert atmosphere, for example nitrogen. The reaction mixture is generally stirred at about room temperature for less than 1 hour such as about 20 minutes followed by heating to about 50° C. To this mixture is added the macrolide of formula (IV), generally as a solution with Pd(PPh₃)Cl₂ in a suitable solvent, such as acetonitrile. The final mixture is then stirred for a period of 2-10 h, such as about 4 h.

Other compounds of formula (I) where the unsaturated bond is not linked directly to the R¹ moiety can be prepared by known methods for example using an aldehyde coupling reaction followed by dehydration of the alcohol product thereof.

Alternatively a metathesis reaction may be used to prepare said unsaturated compounds. For example, the reaction of a compound of formula (IV) where Z* represents C₃₋₆ alkenyl with the alkene group terminal, for example Z* is allyl, with a quinolone of formula (IIa) where p is 0-3, for example 1.

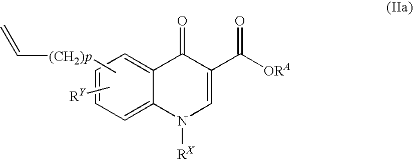

Compounds of formula (IIa) may be prepared from compounds of formula (II) by methods known in the art, for example treatment with bis(dibenzylideneacetone)palladium and Ph₃P and allyltrimethylsilane in a suitable solvent such as DMF, at a suitable temperature such as 50° C., gives a compound of formula (IIa) where p=1.

Compounds of formula (II) are either commercially available or can be readily prepared. For example, compounds of formula (II) wherein L is attached at the 6-position can be prepared as shown in scheme 1 below wherein R^{A} is C₁₋₆alkyl, L is iodo and R^{X} and R^{Y} are as defined above for compounds of formula (I).

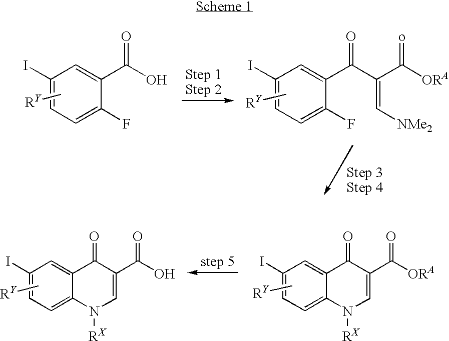

Other compounds of formula (II) may be prepared by analogous methods.

Step 1 the acid is converted to an acid chloride using a reagent such as phosgene, oxallyl chloride or thionyl chloride with DMF as catalyst at a temperature of 0-40° C., typically 20° C. for about 3 h in a solvent such as DCM

In step 2) reaction with ethyl 3-(dimethylamino)acrylate may be effected in DMF or another suitable high boiling solvent at a temperature of 60-120° C. typically 90° C. for about 2.5 h.

In step 3) reaction with R^{x}NH₂ occurs at temperatures between 0-40° C., typically 20° C. for about 1.5 h.

In step 4) cyclisation takes place in DMF or another suitable high boiling solvent in the presence of a base such as potassium carbonate at a temperature of 50-120° C. typically 70° C. for about 2 h.

In step 5) de-esterification may be accomplished by methods known in the art, for example treatment of the ester with LiOH in THF at room temperature.

Other compounds of formula (II) can be prepared by analogous methods as described in J. Med. Chem. 1995, 38, 973 and Aust. J. Chem., 1973, 26, 907.

Guidance for the preparation of compounds of formula (II) wherein Rx represents C₁₋₆alkyl can be obtained from WO 2004/101587 and WO 2004/101586.

Guidance for the preparation of compounds of formula (II) wherein R^{X} represents C₃₋₆cycloalkyl can be obtained from WO2004/101590 and WO 2004/101586.

Compounds of formula (III), wherein Y represents oxygen, L is iodo in the quinolone 6-position and R^{Y} is hydrogen can be prepared according to the general method shown in scheme 2 below.

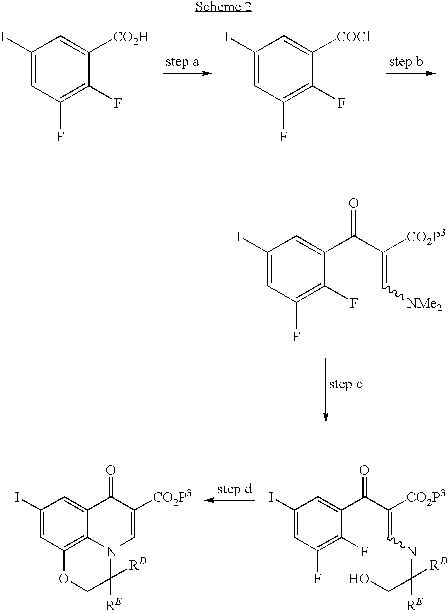

wherein:
P³ represents a carboxylic acid protecting group, for example methyl, ethyl or benzyl such as ethyl, and
R^{D} and R^{E} are as defined above for compounds of formula (I).

Step a typically involves the reaction of the acid with oxalyl chloride in DCM using DMF as catalyst at room temperature for 1-2 h.

Step b involves the reaction of the product form step a with ethyl 3-(dimethylamino)acrylate in toluene and a hindered organic base such as triiethylamine at elevated temperatures, typically reflux for about 2 hours.

Step c involves the reaction of the product from step b with an optionally substituted aminoethanol in ethanol at room temperature for about 1-2 h.

Step d involves the double cyclisation of the product from step c using a strong base such as DBU in a polar solvent such as DMF at elevated temperatures, typically 60° C. for about 16-20 h.

Compounds of formula (III), wherein X represents oxygen, L is iodo in the quinolone 6-position, R^{Y} is hydrogen and R^{D} and R^{E} independently represent hydrogen or methyl, can be prepared according to the general method shown in scheme 3 below.

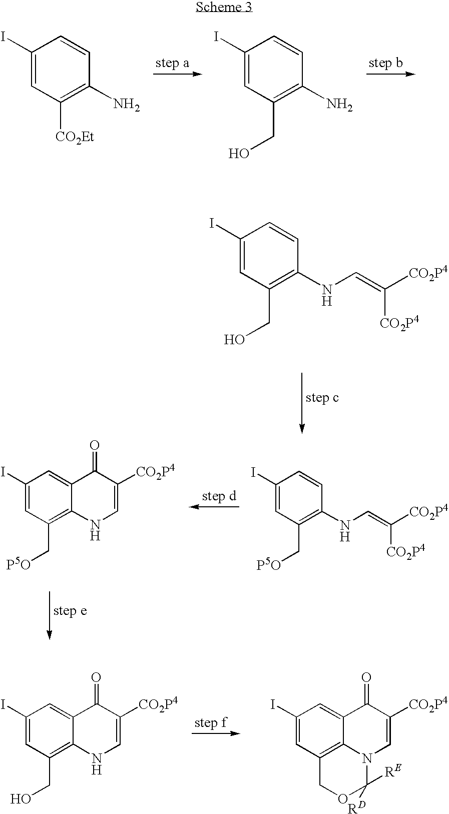

wherein:
P⁴ represents a carboxylic acid protecting group, for example methyl, ethyl or benzyl such as ethyl, and
P⁵ represents an alcohol protecting group such as acetyl.

Step a involves the reduction of the ester function with a reducing agent such as di-isobutyl aluminium hydride in an etheral solvent, typically THF at −30 to −15° C. for 18 h.

Step b involves the reaction of the product from step a with diethyl ethoxymethylene malonate at elevated temperature, typically 100-140° C. for about 2-10 h.

Step c involves the treatment of the product from step b with an acetylating agent in the presence of base in an appropriate solvent typically acetyl chloride, triethylamine in DCM at room temperature.

Step d involves heating the product from step c in an appropriate solvent, typically at 230-260° C. in diphenylether for about 0.5-2 h.

Step e involves the hydrolysis of the acyl group in the product from step d typically by reaction with sodium ethoxide in ethanol at room temperature for 10-20 h.

Step f involves the reaction of the product from step e with a dihalomethane in the presence of base, typically chloroiodomethane and potassium carbonate in DMF at 80° C. for 18 h or a dialkoxyalkane under acid catalysis, typically 2,2-dimethoxypropane and 4-toluene sulfonic acid monohydrate in NMP 80° C. for 1.5 h.

Compounds of formula (III), wherein both Y and X are —CH₂—, R^{Y} is hydrogen and L is bromo in the quinolone 6-position can be prepared by the general method set out in scheme 4 below.

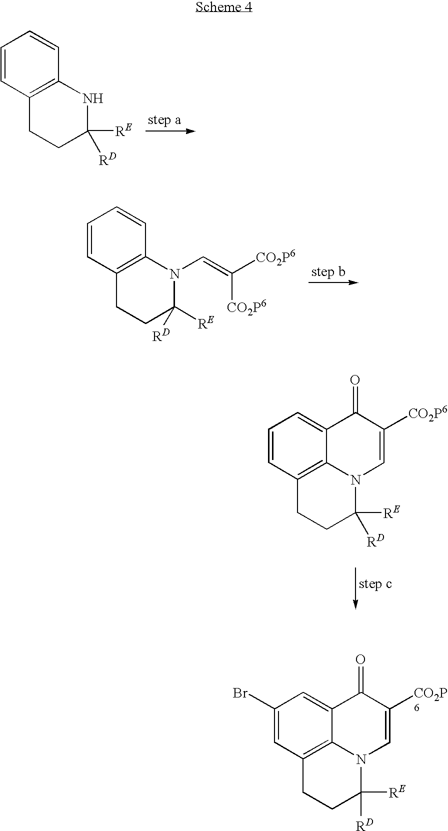

wherein:
P⁶ represents a carboxylic acid protecting group, for example methyl, ethyl or benzyl such as ethyl.

Step a involves the reaction of a 1,2,3,4-tetrahydroquinoline with diethyl ethoxymethylene malonate at elevated temperature, typically 100-150° C. for 1 h.

Step b involves the cyclisation of the product from step a by heating in polyphosphoric acid, typically at 100-150° C. for 4 h.

Step c involves brominating the product of step b, typically with bromine in acetic acid at room temperature for 4 h.

In schemes 2, 3 and 4 above the ester protecting groups may be chosen from a number of alternatives but benzyl protection may not be compatible with strongly acid conditions, such as polyphosphoric acid.

General guidance of the preparation of compounds of formula (III) wherein X and Y represent CH₂ can be obtained from WO2004/101587. General guidance in the preparation of cyclised compounds can be obtained from US patent application publication number 2002/0025959.

Compounds of the general type (IV), wherein Z* is alkenyl, can be prepared using the general methodology in step d of scheme A and step c of scheme B above.

Compounds of formula (IV) may also be prepared by the general process below, which comprises:

a) reacting a compound of formula (V)

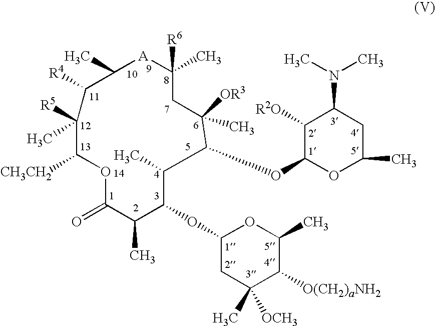

or a protected derivative thereof wherein:
R² represents a hydroxyl protecting group such as acetyl,
R³, R⁴, R⁵, R⁶, A and a are as defined above for compounds of formula (Ia), with an alcohol as shown below:

Z^{A}(CH₂)₁₋₄OH
wherein:
Z^{A} represents HC≡C— or CH₂═CH—,
to provide the required product, and
   b) optionally followed by removal of one or more protecting groups.

The reaction may be effected in an inert atmosphere in a suitable solvent in the presence of an acid and nitrite salt.

More specifically the reaction may be effected under an inert atmosphere such as nitrogen, in a suitable solvent, for example, propyn-1-ol also known as propargyl alcohol (for products of the reaction wherein Z* is —CH₂C≡CH) or allyl alcohol (for products of the reaction wherein Z* is CH₂C═CH₂) and formic acid, by introducing NaNO₂ generally in a portionwise manner, with stirring for a period of approximately 30 mins to 1.5 h after the addition of each portion.

After the reaction has been worked-up deprotection may be effected, for example:
where Z*, in products of the reaction, contains C≡CH by stirring the product of step a in aqueous methanol and aqueous potassium carbonate at about room temperature for about 12-24 h, such as overnight, followed by about 1 h heating at about 55° C., or
where Z*, in products of the reaction, contains CH═CH₂ stirring the product of step a in aqueous methanol and potassium carbonate at about room temperature for about 12-24 h, such as about 17 h.

Compounds of formula (IV) wherein Z* is C₃₋₆alkenyl or C₃₋₆alkynyl can also be prepared by reaction of an alcohol of formula (IX)

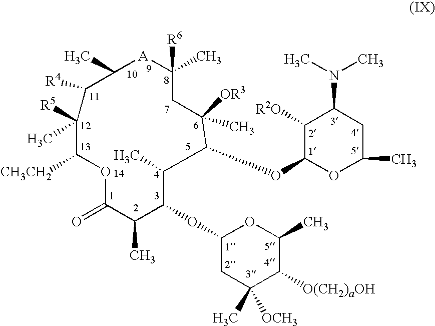

wherein R², R³, R⁴, R⁵, R⁶, A and a are as defined above for compounds of formula (Ia), with a moiety of formula (X)

L′—(CH₂)_{q}—CH═CH₂ or L′—(CH₂)_{q}—C≡CH (X)

wherein L′ is a leaving group such as chloro, bromo, iodo or mesylate and q is an integer from 1-4, in a suitable solvent such as THF, in the presence of a base such as NaH.

Alternatively, compounds of formula (IV) may be prepared by treatment of (IX) with a suitable reagent such as allyl t-butyl carbonate in the presence of a catalyst such as (Ph₃P)₄Pd.

Compounds of formula (IX) may be prepared from compounds of formula (VIa) via reduction with, for example, sodium borohydride, or from compounds of formula (VIII) via hydroboration.

Alternatively compounds of formula (IX) may be prepared by reaction of compounds of formula (V) with water. The reaction may be effected in an inert atmosphere in a suitable solvent in the presence of an acid and nitrite salt.

Compounds of formula (V), where a is 3, and R⁴ and R⁵ are as defined above for compounds of formula (I), may be prepared from compounds of formula (VI):

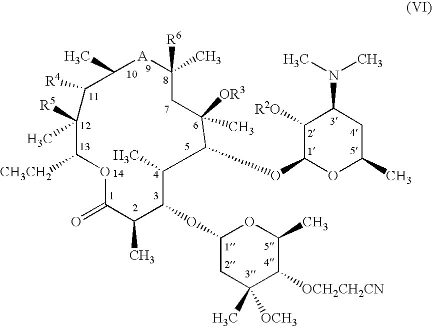

or a protected derivative thereof wherein:
R² represents a hydroxyl protecting group such as acetyl, and
R³, R⁴, R⁵, R⁶ and A are as defined above for compounds of formula (Ia), by treatment with a reducing agent, for example by hydrogenation.

The reduction may be effected by treatment of the compound of formula (VI) in a suitable solvent such as acrylonitrile at a reduced temperature, for example below 10° C., such as about 0° C., and under an inert atmosphere, such as nitrogen.

An example of such a preparation is given in Scheme 5:

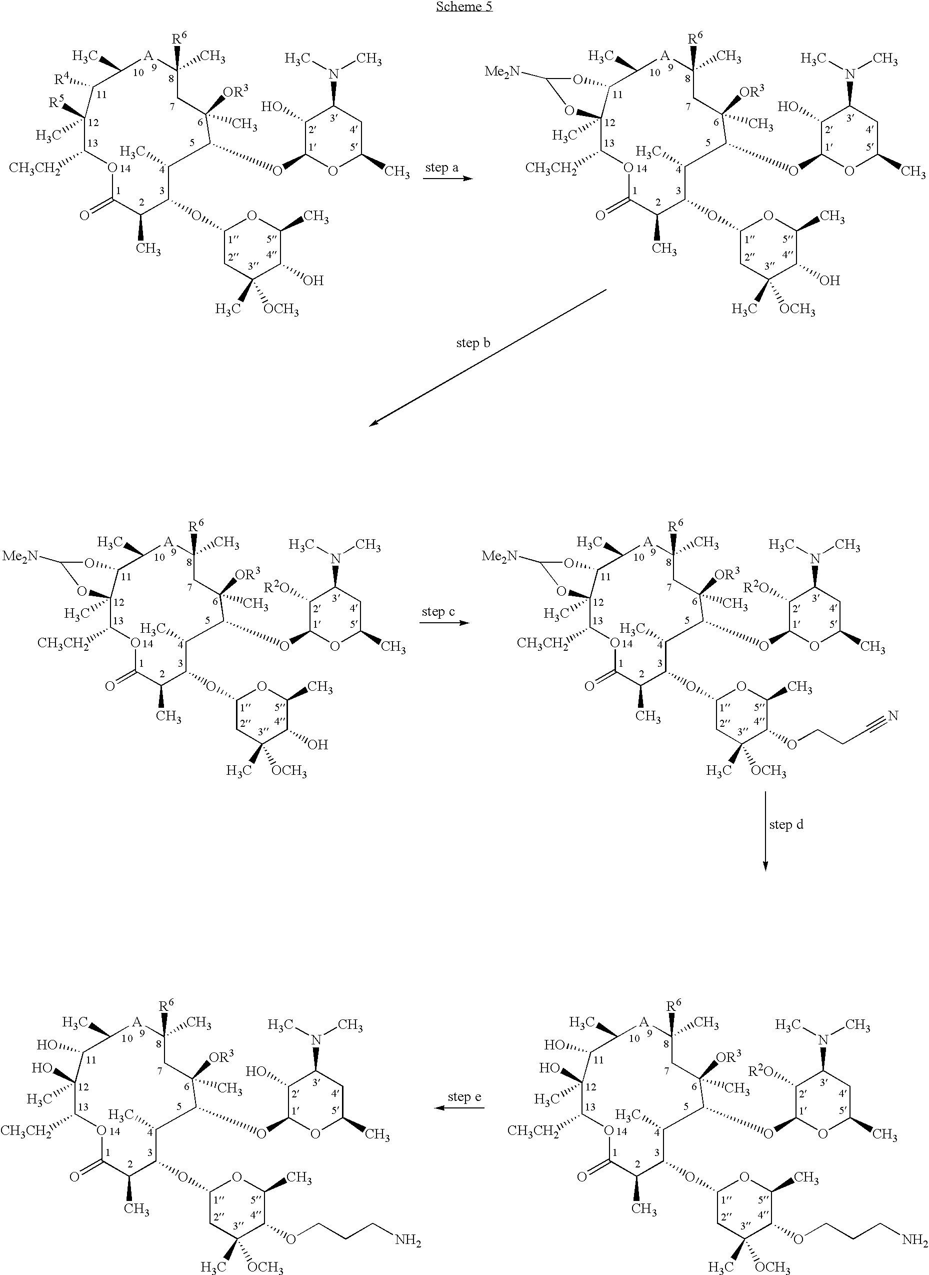

Step a involves the simultaneous protection of the 11 & 12 hydroxy groups in azithromycin by formation of the dimethylformamide cyclic acetal using alcohol exchange with dimethylformamide dimethylacetal at 65° C.

Step b involves protection of the 2′-hydroxy of the product formed in Step a by the formation of the corresponding 2′-acetate, or similar, using acetic or other carboxylic acid anhydrides.

Step c Michael reaction of the 4″-hydroxy group of the product formed in Step b with acrylonitrile under base catalysed conditions to give the corresponding 4″-propionitrile analogue.

Step d involves the simultaneous reduction of the nitrile functionality and cleavage of the 11,12-dimethylamino acetal in the product formed in Step c by catalytic hydrogenation using platinum oxide in acetic acid.

Step e effects removal of the 2′-acetate protecting group by treating the product formed in Step d with aqueous methanol and potassium carbonate at room temperature.

Alternatively compounds of formula (V) may be prepared by reacting a compound of formula (VIa):

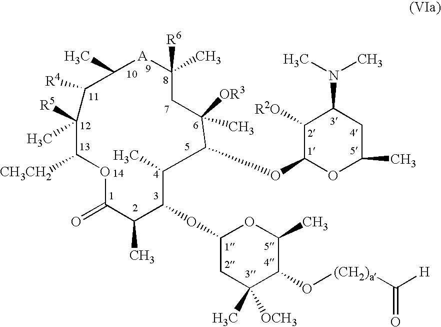

or a protected derivative thereof wherein:
R², R³, R⁴, R⁵, R⁶ and A are as defined above for compounds of formula (Ia) and
a′ represents an integer 1 to 4,
   with ammonia under suitable conditions.

For example, the reaction may be carried out as a reductive amination in a solvent such as methanol and DMF under neutral to mildly acidic conditions. Suitable reducing agents include, for example, sodium cyanoborohydride, sodium triacetoxyborohydride, tetrabutylammonium triacetoxyborohydride or a similar polymer bound borohydride. Sodium borohydride in a solvent such as acetic acid wherein the triacetoxyborohydride is formed in situ may also be employed. Alternatively, palladium on charcoal and hydrogen may be employed to effect the reduction. Suitable reagents for adjusting acidity are acetic acid and sodium acetate.

Specifically, compounds of formula (V) or a protected derivative thereof wherein:
a represents 4 or 5,
R² represents a hydroxyl protecting group such as acetyl,
R⁴ and R⁵ taken together with the intervening atoms form a cyclic group having the following formula:

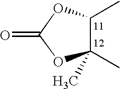

A, R³ and R⁶ are as defined above for compound of formula (I) may be prepared by reacting a compound of formula (VIa) wherein a′ is 2 with (triphenylphosphoranylidene)acetonitrile followed by hydrogenation of double bond and cyano group.

Compounds of formula (VI) may be prepared by reacting a compound of formula (VII):

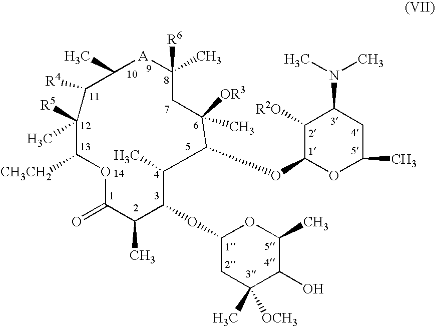

or a protected derivative thereof wherein:
R², R³, R⁴ and A are as defined above for compounds of formula (Ia), and
R⁴ and R⁵ taken together with the intervening atoms form a cyclic group:

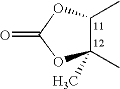

or R⁴ and R⁵, taken together with the intervening atoms form a cyclic group:

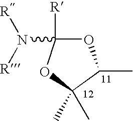

in which R′ represents hydrogen or methyl; R″ and R′″ independently represent C₁₋₃alkyl,
   with acrylonitrile.

A solution of a compound of formula (VII) in acrylonitrile at a reduced temperature such as 0° C. under an inert atmosphere, for example nitrogen, may be treated with a tert-butanol and a strong base, for example sodium hydride. After addition of the reagents the reaction mixture is stirred for about 30 minutes at a reduced temperature such as 0° C. followed by stirring for about 3 h at room temperature.

Compounds of formula (VIa) may be prepared from compounds of formula (VIII):

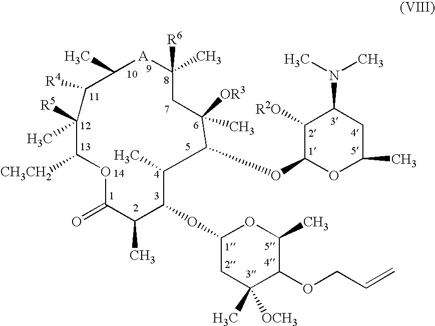

wherein:
R², R³, R⁴, R⁵, R⁶ and A are as defined above for compounds of formula (Ia).

For example, when a′ represents 1, the reaction may be achieved by oxidative cleavage for example using osmium tetroxide and sodium periodate.

Alternatively ozone at a low temperature such as 0° C. or below in the presence of an acid, for example TFA (trifluoroacetic acid) may be employed.

Alternatively, oxidation of the double bond in compounds of formula (VIII) to the corresponding aldehyde of formula (VIa) may be effect in the absence of acid. However, this may result in the oxidation of one or more amino groups in the molecule, for example on the moiety below:

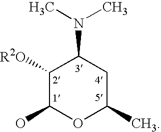

The N-oxide(s) may be reduced at various stages in the general process, as required, for example by treatment with a suitable reducing agent, for example, triphenyl phosphine under appropriate conditions, as described in J. of Antib. 41 (1988) 1029-1047 or with Zn powder as described in EP 0 985 679. Nevertheless it is generally advisable to protect the nitrogens as the salt.

Where a′ is 2, hydroboration of suitably protected compounds of formula (VIII) with 9-BBN (9-borabicyclo[3.3.1]nonane, or other suitable boranes, followed by treatment with peroxide, for example hydrogen peroxide and a base such as sodium hydroxide.

For a′=3 or 4, compounds of formula (VIII) may be chain extended using olefin cross-metathesis (H. E. Blackwell et. al. J. Am. Chem. Soc., 2000, 122, 58-71) with a suitably functionalised olefin, for example but-2-ene-1,4-diol, followed by double bond reduction and oxidation of the terminal alcohol, where appropriate, as under certain conditions the desired aldehyde can be isolated directly.

Compounds of formula (VII) may be prepared by the procedure described by S. Djokic et al. in J. Chem. Res. Synop (S) 1988, 152.

Compounds of formula (VIII) can be formed by palladium-catalysed allylation of suitably protected 4″ hydroxy compounds. Guidance to O-allylation can be gained from Stoner et al J. Org Chem 68, 8847-8852, references therein (palladium catalysed allylation of 6-OH in erythromycin derivatives) and Freiberg et al in U.S. Pat. No. 5,288,709 (4″-O allylation using NaN(TMS)₂/allyl bromide on a highly modified and protected erythromycin derivative).

General guidance for the preparation of compounds wherein R⁴ or R⁵ are hydroxy or R⁴ and R⁵ taken together with the intervening atoms form a cyclic group having the following structure:

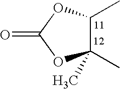

may be gained from EP 0 307 177, WO 00/78773 and WO 97/42204, EP 0 508 699, J. Chem. Res. Synop. (1988, pages 152-153) and U.S. Pat. No. 6,262,030.

Compounds wherein R⁴ and R⁵ taken together with the intervening atoms form a cyclic group having the following structure:

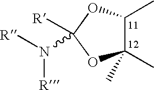

may be prepared from the corresponding diol (R⁴ and R⁵ are both OH) by alcohol exchange with the appropriate acetal, for example dimethylformamide dimethylacetal (R′ is H, R″ and R′″ are both Me) or dimethylacetamide dimethylacetal (R′, R″, and R′″ each represent Me).

All publications, including but not limited to patents and patent applications, cited in this specification are herein incorporated by reference as if each individual publication were specifically and individually indicated to be incorporated by reference herein as though fully set forth.

In order that the invention may be more fully understood the following examples are given by way of illustration only.

The following abbreviations are used in the text: 9-BBN for 9-borabicyclo[3.3.1]nonane, BOC for t-butoxycarbonyl, DBU for 1,8-diazabicyclo[5.4.0]undec-7-ene, DCM for dichloromethane, DMF for N,N-dimethylformamide, DMSO for dimethyl sulfoxide, EtOAc for ethyl acetate, HOAc for acetic acid, EtOH for ethanol, MeCN for acetonitrile, MeOH for methanol, TFA for trifluoroacetic acid, TOTF for trans-di-μ-acetatobis(2-(di-o-tolyl triphenylphosphine, dppb for 1,4-bis(diphenylphosphino)butane, Pd₂(dba)₃ for tris(dibezylideneacetone)dipalladium (0), Pd(PPh₃)₄ for tetrakis-(triphenylphosphin)-palladium, Pd(OAc)₂ for palladium acetate, EDC.HCl for 1-(3-dimethylaminopropyl)-3-ethylcarbodiimide hydrochloride, TEA for triethyl amine, DMAP for N,N-dimethylamino-pyridine, HOBT for 1-hydroxy benzotriazole hydrate, THF for tetrahydrofuran, MgSO₄ for anhydrous magnesium sulphate and Na₂SO₄ for anhydrous sodium sulphate.

In the procedures that follow, reference to an Intermediate by number is typically provided. This is provided merely for assistance to the skilled chemist to identify the starting material used. The starting material may not necessarily have been prepared from the batch referred to. In addition, the preparation of an Example compound is typically presented as a series of individual reaction steps, for example (a), (b), (c), etc. This is also provided merely for assistance to the skilled chemist to identify a suitable sequence of reaction steps to prepare the Example. Although each of the reaction steps indicated will have been carried out as described, the steps (a), (b), (c), etc. may not have been performed in one continuous sequence from the same batch of starting materials.

In the procedures that follow, reference is made to certain compounds being made “according to the procedure”. As is appreciated by those skilled in the art, such analogous processes may involve variations in synthetic procedure, for example in the solvent(s) used for extraction, or in the eluting solvent system used for chromatographic purification.

### EXAMPLES

2′-O-Acetyl-azithromycin and 2′-O-acetyl-azithromycin-11,12-carbonate may be prepared by the procedures described by S. Djokic et al. in J. Chem. Res. (S) 1988, 152.

Reverse phase HPLC refers to the use of an XTerra MS C18 column with a gradient of MeCN containing 0.1% TFA in water containing 0.1% TFA as eluent.

Unless otherwise stated, chromatography was conducted on silica gel.

Mass directed automatic preparative HPLC refers to the use of Waters Atlantis dC18 5 micron columns with a gradient of MeCN containing 0.1% HCO₂H in H₂O containing 0.1% HCO₂H as eluent.

Where Example compounds are isolated as salts these are typically characterised and the stoichiometry determined using proton NMR, for example by considering the chemical shift values, the integrated number of protons, and by assignment of one or more equivalent peak(s) from the acid and from the parent base.

It will be understood that in the context of the examples used to illustrate the invention that information about how the compounds were prepared cannot be drawn from the format used to present the information, for example, the intermediates and final products may have been prepared by different individuals, or at different timepoints, employing appropriate techniques.

### Intermediate 1

### 4″-O-(2-Cyano-ethyl)-2′-O-acetyl-azithromycin-11,12-cyclic carbonate

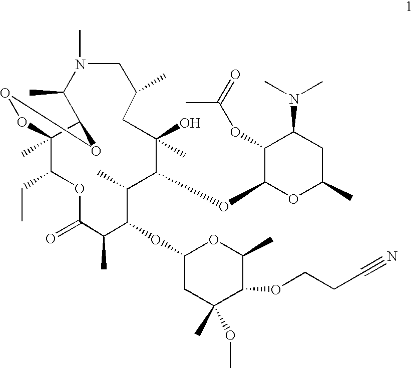

A solution of 2′-O-acetyl-azithromycin-11,12-cyclic carbonate (30 g, 0.037 mol) (may be prepared by the procedures described by S. Djokic et al. in J. Chem. Res. (S) 1988, 152) in acrylonitrile (400 ml, 5.78 mol) was cooled to 0° C. under N₂ atmosphere and tert-butanol (11 ml, 0.11 mol) was added. Then NaH (1.5 g, 60% dispersion in mineral oil) was added portionwise over 1 hour. The reaction mixture was stirred at 0° C. for 30 minutes and for further 3 hours at room temperature. The reaction mixture was filtered and solvent evaporated in vacuum. Then EtOAc (200 ml) was added, filtered once again and the organic layer washed with saturated aqueous NaHCO₃ (200 ml). The organic layer was dried over K₂CO₃, then the solvent was evaporated in vacuum giving crude foamy product which was dissolved in diethyl ether (50 ml) at room temperature and crystallized by stirring at 0° C. After filtration the title compound (11.3 g) was obtained.

MS; m/z (ES): 870.1 [MH]+

### Intermediate 2

### 4″-O-(3-Amino-propyl)-2′-O-acetyl-azithromycin-11,12-cyclic carbonate

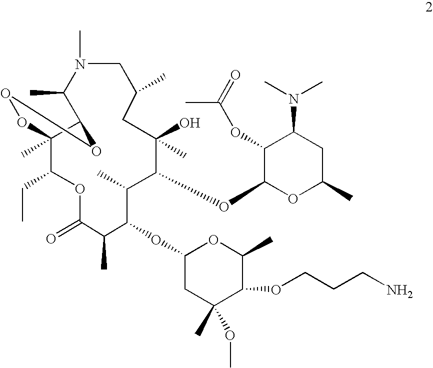

A high pressure reactor was filled with solution of Intermediate 1 (10 g, 11.4 mmol) in acetic acid (150 ml), PtO₂ (1 g) was added and the reaction mixture stirred at 5 bar pressure of H₂ for 24 hours. The catalyst was filtered through celite and the solvent evaporated in vacuum. Material from three similarly carried out batches was combined. The oily product was diluted with DCM (300 ml) and extracted with water (600 ml). The water layer was extracted with DCM (400 ml), and the pH adjusted to 6.2. The water layer was extracted a second time with DCM (200 ml). The combined organic layers (at pH 6.2) were dried over K₂CO₃ and evaporated in vacuum yielding the title compound (21.2 g).

MS; m/z (ES): 874.1 [MH]⁺

¹³C-NMR (125 MHz, CDCl₃) δ: 176.92 (C-1), 169.59 (2′OAc, C═O), 153.07 (11,12-CK), 99.54 (C-1′), 94.67 (C-1″), 87.26 (C-4″), 85.89 (C-11), 84.49 (C-12), 83.21 (C-5), 76.89 (C-3), 75.73 (C-13), 73.56 (C-3″), 73.06 (C-6), 72.55 (CH₂), 71.47 (C-2′), 67.72 (C-5′), 67.39 (C-9), 64.09 (C-5″), 62.83 (C-3′), 60.93 (C-10), 49.20 (3″OMe), 44.42 (C-2), 43.55 (C-7), 40.88 (C-4), 40.37 (3′NMe₂), 39.42 (CH₂), 35.07 (C-2″), 34.07 (9aNMe), 31.43 (CH₂), 30.79 (C-4′), 26.43 (6Me), 25.92 (8H), 21.77 (8Me), 21.73 (C-14), 21.69 (C-3″), 21.41 (C-5′), 20.93 (2′0Ac, CH₃), 18.10 (5″Me), 14.39 (2Me), 13.47 (12Me), 11.39 (4Me), 10.03 (15Me), 4.84 (10Me).

### Intermediate 3

### 4″-O-(3-Amino-propyl)-2′-O-acetyl-azithromycin-11,12-cyclic carbonate HCl Salt

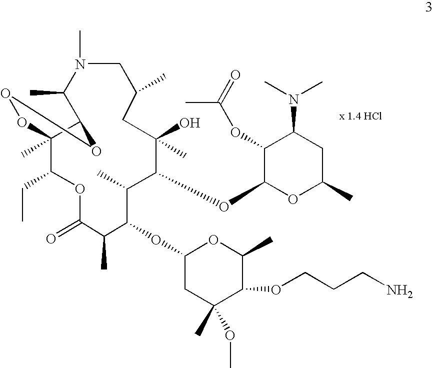

Intermediate 2 (2 g, 2.3 mmol) was dissolved in 2-propanol (12 ml) and then 5-6 N HCl solution in 2-propanol (0.66 ml, 3.3 mmol) was added. The product was precipitated by addition of mixture diisopropyl ether/n-hexane=1:2 (90 ml), and filtered off, drying at 35° C. for 3 hours, yielding the title compound as a white solid (1.9 g).

### Intermediate 4

### 2′-O-acetyl-4″-O-(3-allyloxy-propyl)-azithromycin-11,12-cyclic carbonate

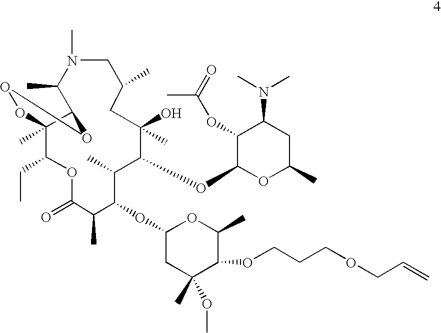

To a solution of Intermediate 3 (0.8 g, 0.92 mmol) in allyl alcohol (10 ml, 0.15 mol), formic acid (0.3 ml, 7.9 mmol) was added under flow of N₂ atmosphere. Then NaNO₂ (0.4 g, 5.8 mmol) was added in portions over 15 minutes and the reaction mixture stirred at room temperature for 5 hours. Another portion of NaNO₂ (0.2 g; 2 equiv.) was added and stirring continued overnight. The reaction mixture was filtered and the solvent evaporated in vacuum. To the residue EtOAc (20 ml) was added and washed with saturated aqueous NaHCO₃ (2×30 ml). Evaporation of the organic layer yielded the title compound as a yellow foam (0.86 g).

MS; m/z (ES): 915.5 [MH]⁺

### Alternative Preparation Intermediate 4

### Step a) Intermediate 5

### 2′-O-Acetyl-4″-O-(3-hydroxy-propyl)-azithromycin-11,12-cyclic carbonate

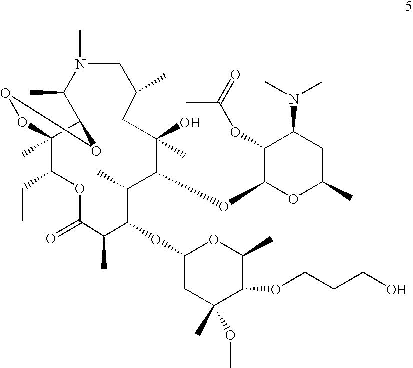

Intermediate 2 (4.7 g, 5.4 mmol) was dissolved in 10% HOAc (30 ml), and cooled to 0° C. To this solution NaNO₂ (2.4 g, 35 mmol) was added over 1 hour and the reaction mixture was stirred at room temperature for 2.5 hours. The reaction mixture was diluted with water (50 ml), DCM was added (70 ml) and pH adjusted to 10.5. The organic layer was dried over K₂CO₃ and evaporated in vacuum yielding the crude product (4.5 g) as a white foamy solid which was combined with material from a second batch prepared from Intermediate 2 (3.7 g) in a similar manner. Thus, the combined material (4.5 g+3.5 g) was purified by column chromatography (EtOAc/n-hexane/Et₃N=10:10:2) yielding the crude product (3.1 g). The Et₃N was removed by extraction using EtOAc (40 ml) and saturated NaHCO₃ (3×30 ml). EtOAc layer was dried over K₂CO₃ and evaporated in vacuum yielding the title compound (2.94 g).

MS; m/z (ES): 875.6 [MH]⁺

¹³C-NMR (75 MHz, CDCl₃) δ ppm: 177.16, 170.36, 153.39, 100.11, 94.87, 87.24, 86.51, 84.83, 84.17, 77.54, 76.54, 74.69, 73.78, 73.44, 68.02, 67.41, 64.46, 63.42, 63.02, 61.62, 49.42, 45.07, 42.91, 42.45, 41.91, 34.99, 34.65, 32.44, 30.61, 27.06, 26.28, 22.26, 22.19, 22.09, 21.38, 18.38, 14.97, 13.86, 10.54, 10.01, 5.30.

### Step b) Intermediate 4

### 2′-O-acetyl-4″-O-(3-allyloxy-propyl)-azithromycin-11,12-cyclic carbonate

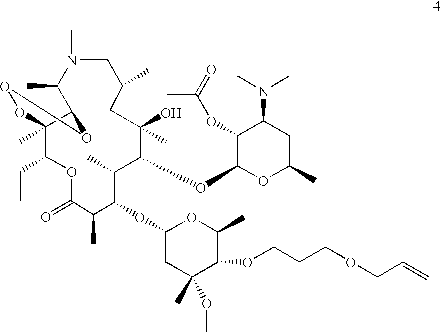

To a mixture of allyl-tert butyl carbonate (6 ml, 38 mmol) and Intermediate 5 (1 g, 1.1 mmol.), under an atmosphere of nitrogen, catalysts Pd₂(dba)₃ (0.12 g, 0.11 mmol) and dppb (0.12 g, 0.28 mmol) were added and the reaction mixture was stirred at 80° C. for 2 hours. Then Pd(PPh₃)₄ (0.27 g. 0.23 mmol) was added and stirring continued for 1.5 hours and left at room temperature overnight. The reaction mixture was diluted with EtOAc (50 ml), filtered and evaporated in vacuum. To the residue EtOAc (50 ml) was added and extracted with water (100 ml) at pH 3. Then to the water layer EtOAc (80 ml) was added and pH adjusted to 8.5. The leyers were separated, organic one dried over K₂CO₃ and evaporated in vacuum yielding the title compound (1 g) as a foamy solid.

MS; m/z (ES): 915.6 [MH]⁺ (60%)

### Intermediate 6

### 4″-O-(3-Allyloxy-propyl)-azithromycin

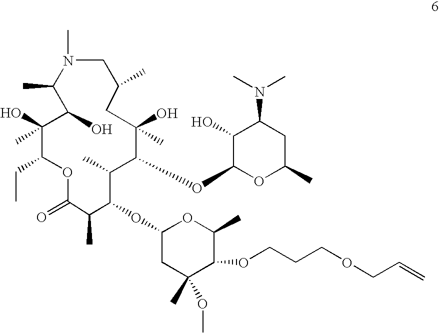

To a solution of Intermediate 4 (0.86 g, 0.94 mmol) in MeOH/H₂O=2/1 (45 ml), K₂CO₃ (1.95 g, 14 mmol.) was added and the reaction mixture stirred at room temperature for 17 hours. After evaporation of MeOH, EtOAc (40 ml) was added to the residue and washed with saturated aqueous NaHCO₃ (2×30 ml). The organic layer was evaporated and the crude product purified by column chromatography (eluent DCM/MeOH/NH₃=90:9:1.5) yielding the title compound (0.25 g).

MS; m/z (ES): 847.4 [MH]⁺

### Intermediate 7

### 2′-O-Acetyl-4″-O-(3-prop-2-ynyloxy-propyl)-azithromycin-11,12-cyclic carbonate

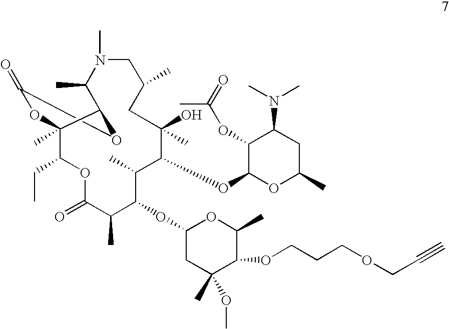

To a solution of Intermediate 2 (13.6 g, 15.6 mmol) in propargyl alcohol (85 ml, 1.5 mol), HCOOH (2.7 ml, 72 mmol) was added under N₂ flow and cooled to 0° C. Then NaNO₂ (6.5 g, 94 mmol) was added portionwise over 45 minutes and the reaction mixture stirred at room temperature for 3 hours. After another portion of NaNO₂ (2 equiv.) was added stirring was continued for 2 hours. The reaction mixture was diluted with EtOAc (70 ml) and extracted with saturated aqueous NaHCO₃ (3×80 ml). Evaporation of the organic layer yielded the title compound (13.9 g) as a yellow foam.

MS; m/z (ES): 913.1 [MH]⁺

### Intermediate 8

### 4″-O-(3-Prop-2-ynyloxy-propyl)-azithromycin)

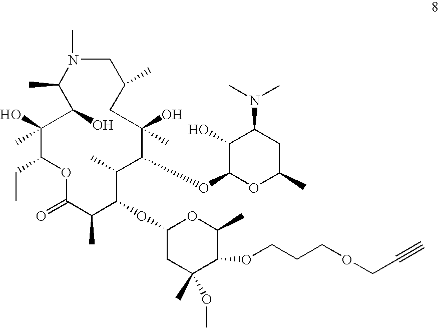

To a solution of Intermediate 7 (13.9 g, 15.2 mmol) in MeOH/H₂O=2/1 (600 ml), K₂CO₃ (30 g, 0.22 mol) was added and the reaction mixture stirred at room temperature overnight and one hour at 55° C. Then MeOH was evaporated and EtOAc (150 ml) was added to the residue and washed with saturated aqueous NaHCO₃ (200 then 100 ml). The organic layer was dried over K₂CO₃, then the solvent evaporated to give raw material (12.1 g). The second batch starting from Intermediate 7 (9 g) was prepared by the same procedure except that reaction was conducted by stirring at 55° C. for 3 hours to give the raw product (7.7 g). The combined raw materials (12.1 g+7.7 g) were purified by column chromatography (eluent: DCM/MeOH/NH₃=90:9:0.5) yielding the title compound (8.2 g, 55% purity by LC/MS) which was used in further steps without further purification.

MS; m/z (ES): 845.3 [MH]⁺

### Intermediate 9

### Ethyl 3-(dimethylamino)-2-(2-fluoro-5-iodobenzoyl)-2-propenoate

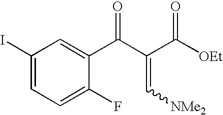

A stirred suspension of 2-fluoro-5-iodobenzoic acid (28.1 g) in DCM (300 mL) at 20° C. was treated with oxalyl chloride (13.9 mL) and DMF (5 drops). After 1.5 h the clear solution was evaporated and re-evaporated from toluene (2×) under reduced pressure. The acid chloride was re-dissolved in toluene (500 mL) and treated with triethylamine (22.5 mL) and ethyl 3-(dimethylamino)acrylate (19.95 g). After stirring for 1.5 h at 90° C. the mixture was filtered and the solution flash chromatographed on silica gel eluting with 40 to 70% EtOAc in light petroleum 40-60° C. to give the title compound (30.8 g); APCI m/z 392.1 [M+H]⁺.

### Intermediate 10

### Ethyl 3-(2,2-dimethylhydrazino)-2-(2-fluoro-5-iodobenzoyl)-2-propenoate

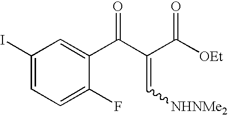

A stirred suspension of ethyl 3-(dimethylamino)-2-(2-fluoro-5-iodobenzoyl)-2-propenoate (28.2 g) in EtOH (300 mL) was treated with 1,1-dimethylhydrazine (4.76 mL). After stirring overnight the clear solution was evaporated under reduced pressure to give the title compound (29.6 g); APCI m/z 407.0 [M+H]⁺.

### Intermediate 11

### Ethyl 1-(dimethylamino)-6-iodo-4-oxo-1,4-dihydro-3-quinolinecarboxylate

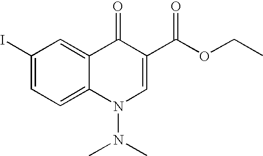

A mixture of ethyl 3-(2,2-dimethylhydrazino)-2-(2-fluoro-5-iodobenzoyl)-2-propenoate (28.5 g) and potassium carbonate (14.5 g) in DMF (300 mL) was stirred at 100° C. for 1 h and then cooled to 20° C. The mixture was poured into water, the solid filtered off, washed with water and dried to give the title compound (22.8 g); APCI m/z 387.0 [M+H]⁺.

### Intermediate 12

### Ethyl 1-ethyl-6-iodo-4-oxo-1,4-dihydro-3-quinolinecarboxylate

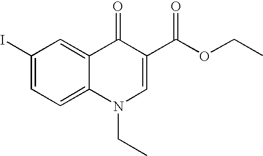

A mixture of ethyl 6-iodo-4-oxo-1,4-dihydro-3-quinolinecarboxylate (WO1999/32450) (25 g) and potassium carbonate (21 g) in dry DMF (100 mL) was heated at 60° C. After 15 min iodoethane (24 mL) was added and the resultant mixture stirred for 24 h. After cooling the volume was reduced by approximately ⅔ and the residue poured into water (500 mL). The mixture was stirred for 0.5 h and then the precipitated solid was filtered under suction and dried under vacuum over phosphorus pentoxide for 10 h to yield the title compound (26.5 g) as a white solid, ¹H NMR δ (CDCl₃) 1.41 (3H, t, J=7.1 Hz), 1.53 (3H, t, J=7.3 Hz), 4.23 (2H, q, J=7.2 Hz), 4.40 (2H, q, J=7.1 Hz), 7.21 (1H, d, J=8.9 Hz), 7.93 (1H, dd, J=2.1 & 8.9 Hz), 8.47 (1H, s), 8.81 (1H, d, J=2.1 Hz).

### Intermediate 13

### 2′-O-Acetyl-4″-O-allyl-azithromycin 11,12-carbonate

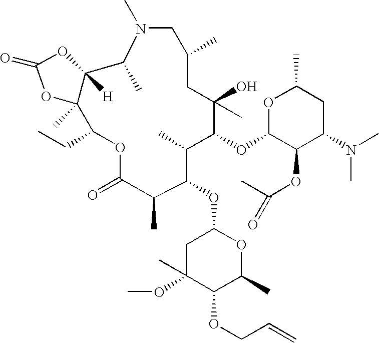

2′-O-Acetyl-azithromycin-11,12-carbonate (67.82 g, 83 mmol) in dry THF (600 mL) was treated with allyl t-butylcarbonate (50 g, 0.315 mol) and tetrakis(triphenylphosphine)-palladium (1.5 g 1.3 mmol). The resultant mixture was heated at 75° C. under argon. After 16 h the reaction was cooled, evaporated under reduced pressure and the residue purified by chromatography on silica eluting with 0-10% (9:1 MeOH/0.880 NH₃) in DCM gave the title compound; ESMS m/z 857.7 [M+H]⁺.

### Intermediate 14

### 2′-O-Acetyl-4″-O-(2-oxoethyl)-azithromycin-11,12-carbonate

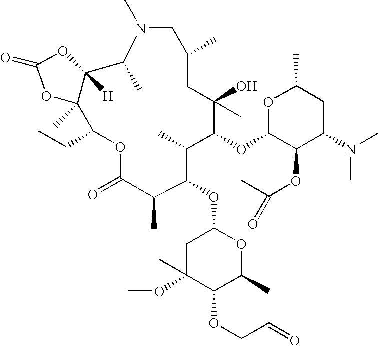

2′-O-Acetyl-4″-O-allyl-azithromycin 11,12-carbonate (9.0 g) in DCM (200 mL) and methanol (20 mL) was cooled to −78° C. with argon bubbled through the solution. TFA (3.2 mL) was added. Ozone was bubbled through until a blue colour developed (1 h). Argon was bubbled through the mixture to flush out the ozone, then dimethyl sulfide (3.1 mL) and triethylamine (6.6 mL) were added. The reaction was stirred at −78° C. for 15 min then removed from the cooling bath and warmed to room temperature. The reaction mixture was washed successively with a saturated aqueous solution of sodium bicarbonate, water and brine then dried and evaporated under reduced pressure to dryness to give crude title compound (9.5 g) which was used without purification.

### Intermediate 15

### 2′-O-Acetyl-4″-O-(2-hydroxyethyl)-azithromycin 11,12-carbonate

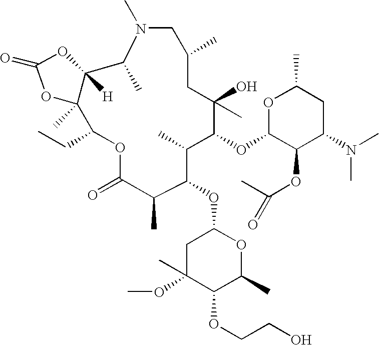

2′-O-Acetyl-4″-O-(2-oxoethyl)-azithromycin-11,12-carbonate (5.0 g) was dissolved in methanol (75 mL) and cooled in an ice-bath before the addition of sodium borohydride (1.25 g). After 1 h water (5 mL) was added and the mixture evaporated under reduced pressure to dryness. The residue was partitioned between brine and DCM. The organic layer was dried, evaporated under reduced pressure and the residue chromatographed eluting with 0-7.5% methanol in DCM to give the title compound (2.8 g); ESMS m/z 861.5 [M+H]⁺.

### Intermediate 16

### 2′-O-Acetyl-4″-O-(2-allyloxyethyl)-azithromycin 11,12-carbonate

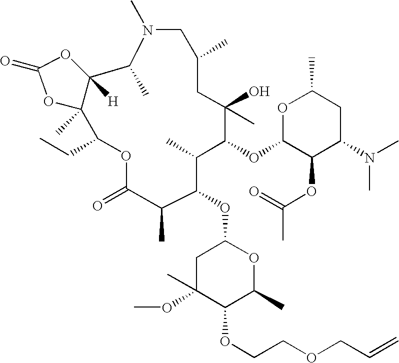

2′-O-Acetyl-4″-O-(2-hydroxyethyl)-azithromycin 11,12-carbonate (0.67 g) in dry THF (1 mL) under argon was treated with allyl t-butyl carbonate (1.2 g) and tetrakis(triphenylphosphine)palladium (0.15 g). The resultant mixture was heated at reflux for 0.75 h. After cooling the reaction mixture was evaporated under reduced pressure and the residue chromatographed eluting with 0-7% methanol in DCM to give 2′-O-acetyl-4″-O-(2-allyloxycarbonyloxyethyl)-azithromycin 11,12-carbonate which was dissolved in dry THF (1 mL) under argon, treated with allyl t-butyl carbonate (1.5 mL) and tetrakis(triphenylphosphine)palladium (0.15 g). The resultant mixture was heated at reflux for 2 h. After cooling the reaction mixture was evaporated under reduced pressure and the residue chromatographed eluting with 0-5% methanol in DCM to give the title compound which was dried in vacuo for 2 days (0.44 g); ESMS m/z 901.9 [M+H]⁺.

### Intermediate 17

### 2′-O-Acetyl-4″-O-(3-oxo-propyl)-azithromycin-11,12-cyclic carbonate

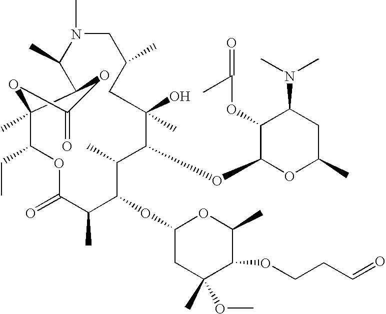

To the degassed solution of Intermediate 5 (0.42 g, 0.48 mmol) in DCM (5 ml) Dess-Martin reagent (0.225 g, 0.53 mmol) was added. Reaction mixture was stirred for 2 hours at room temperature, then the Dess-Martin reagent was filtered off and residue extracted with saturated NaHCO₃ solution (10 ml). The organic layer was dried over K₂CO₃ and evaporated in vacuum yielding the title product (0.4 g).

MS; m/z (ES): 873.6 [MH]⁺.

### Intermediate 18

### 2′-O-Acetyl-4″-O-(4-cyano-but-3-enyl)-azithromycin-11,12-cyclic carbonate

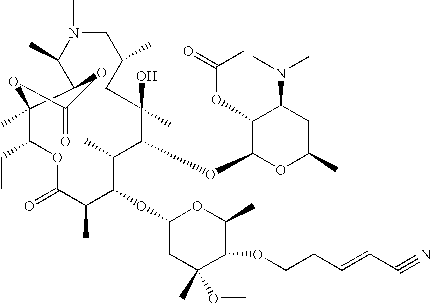

To the degassed solution of Intermediate 17 (0.85 g, 1.02 mmol) in toluene (5 ml) (triphenylphosphoranylidene)acetonitrile (0.4 g, 1.33 mmol) was added. The reaction mixture was stirred for 90 minutes at room temperature, solvent evaporated, residue dissolved in EtOAc (30 ml), washed with water (2×20 ml), and then with saturated NaHCO₃ (2×20 ml) solution. The organic layer was dried over K₂CO₃ and evaporated in vacuum yielding the title product (1.1 g) as yellow oil.

MS; m/z (ES): 896.8 [MH]⁺.

¹³C-NMR (125 MHz, CDCl₃) δ ppm: 176.29, 169.05, 152.41, 151.51, 116.21, 100.64, 98.83, 94.01, 87.37, 85.32, 83.83, 82.59, 76.24, 75.19, 72.90, 72.42, 70.73, 66.82, 63.30, 60.37, 48.64, 43.86, 42.20, 40.40, 34.58, 33.48, 33.13, 25.85, 25.26, 21.12, 21.08, 20.89, 20.37, 17.53, 13.84, 12.83, 10.10, 9.43, 4.21.

### Intermediate 19

### 2′-O-Acetyl-4″-O-(5-aminopentyl)-azithromycin-11,12-cyclic carbonate

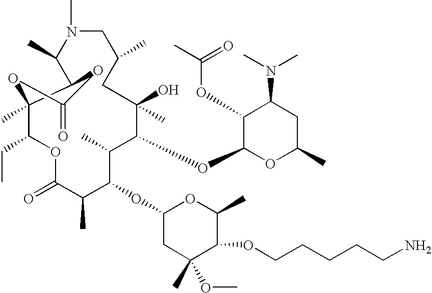

Reduction of Intermediate 18 (1.1 g, 1.28 mmol) in glacial CH₃COOH (30 ml) with PtO₂ (0.45 g) in Parr apparatus at 5 bars for 18 hours resulted with title product (0.42 g).

MS; m/z (ES): 903.1 [MH]⁺.

### Intermediates 20 and 21

### 2′-O-Acetyl-4″-O-(5-hydroxypentyl)-azithromycin-11,12-cyclic carbonate (Intermediate 20)

### 2′-O-Acetyl-4″-O-(pent-4-enyl)-azithromycin-11,12-cyclic carbonate (Intermediate 21)

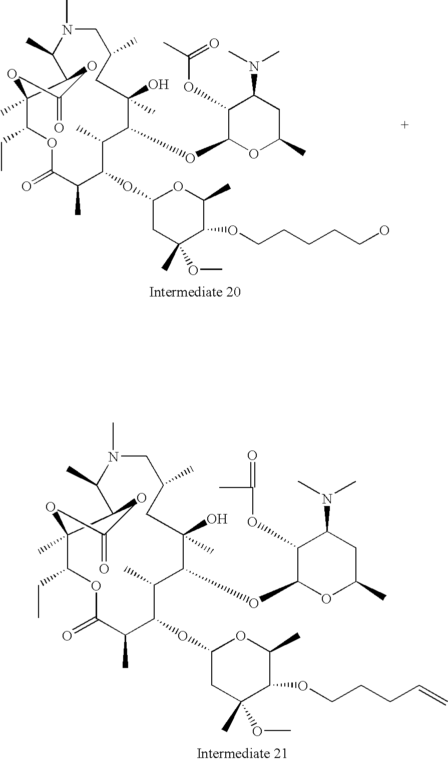

To the solution of Intermediate 19 (0.1 g, 0.17 mmol) in 10% HOAc in H₂O (1.1 ml), NaNO₂ (0.077 g, 1.12 mmol) was added portionwise at 0° C. After stirring for 7 hours, the reaction mixture was left in fridge at 4° C. overnight. Then additional portions of NaNO₂ (0.077 g) and 10% HOAc (1.1 ml) were added. The reaction mixture was left overnight at 4° C. To the reaction mixture DCM was added, pH adjusted 10.7, layers were separated, organic one dried over K₂CO₃ and evaporated under reduced pressure yielding the mixture of Intermediates 20 and 21 (0.06 g). Products were separated by column chromatography (EtOAc/n-hexane/Et₂N=10:10:2) yielding Intermediate 20 (12 mg) and Intermediate 21 (10 mg).

### Intermediate 20

MS; m/z (ES): 904.1 [MH]⁺

### Intermediate 21

MS; m/z (ES): 886.1 [MH]⁺.

### Intermediate 22

### 2′-O-Acetyl-4″-O-(5-allyloxy-pentyl)-azithromycin-11,12-cyclic carbonate

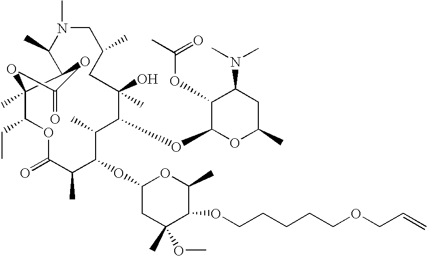

To the degassed suspension of Intermediate 20 (0.125 mg, 0.14 mmol) in allyl t-butyl carbonate (0.642 ml, 4.06 mmol), dppb (0.015 g, 0.014 mmol) was added, then after 15 minutes of stirring Pd₂ (dba)₃ (0.015 g, 0.035 mmol) was added and stirred at 80° C. for 3 hours. Then Pd(PPh₃)₄ (0.032 g, 0.028 mmol) was added, the reaction mixture stirred at 80° C. for additional 2 hours and left overnight at room temperature. The reaction mixture was diluted with EtOAc and purified by column chromatography (eluted first with n-hexane (100 ml), and then with EtOAc (100 ml)). Evaporation of EtOAc eluent gave the title compound (71 mg).

MS; m/z (ES): 944.1 [MH]⁺.

### Intermediate 23

### 2′-O-Acetyl-4″-O-(4-oxobutyl)-azithromycin-11,12-cyclic carbonate

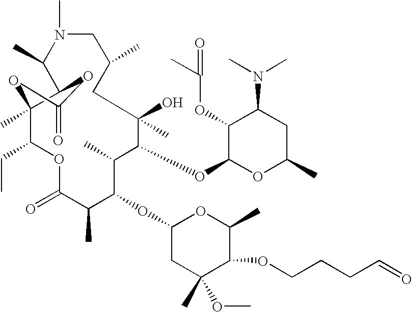

Starting from Intermediate 21 according to the procedure described for Intermediate 14 the title compound is obtained.

### Intermediate 24

### 2′-O-Acetyl-4″-O-(4-hydroxybutyl)-azithromycin-11,12-cyclic carbonate

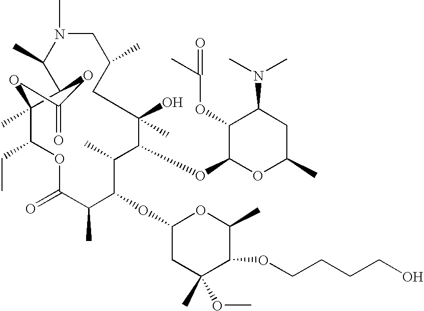

Starting from Intermediate 23 according to the procedure described for Intermediate 15 the title compound is obtained.

### Intermediate 25

### 4″-O-(4-Allyloxybutyl)-azithromycin

### a) 2′-O-Acetyl-4″-O-(4-allyloxybutyl)-azithromycin-11,12-cyclic carbonate

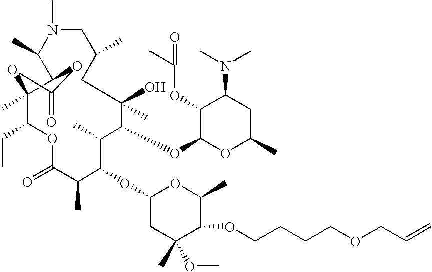

Starting from Intermediate 24 according to the procedure described for Intermediate 4 the title compound is obtained.

### b) 4″-O-(4-Allyloxybutyl)-azithromycin

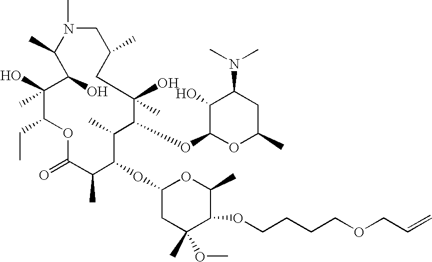

Starting from Intermediate 25a according to the procedure described for Intermediate 6 the title compound is obtained.

### Intermediate 26

### 2-Amino-5-iodo-benzoic Acid methyl ester

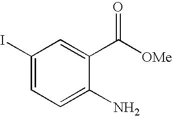

2-Amino-5-iodo-benzoic acid (10.00 g) was dissolved in methanol (200 mL). To this solution, Amberlist-15 (16.00 g) was added and the resulting mixture was heated under reflux under nitrogen for 48 h. After cooling to room temperature the mixture was filtered and the solid was washed with 5% 20M ammonia/MeOH (2×100 mL). The organic phase was dried and the solvent evaporated in vacuo. The residue was purified by flash chromatography (silica gel, ethyl acetate/hexane from 1/15 to 1/12) to give the title compound (7.00 g); ESMS m/z 277.9 [M+H]⁺.

### Intermediate 27

### (2-Amino-5-iodo-phenyl)-methanol

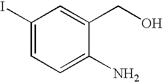

2-Amino-5-iodo-benzoic acid methyl ester (14.00 g) was dissolved in dry THF (50 mL) and 1M diisobutylaluminium hydride in THF (165 mL) was added dropwise to this solution at −30° C. The solution was then stirred at −15° C. for 1 h. To this mixture MeOH (50 mL) was added portionwise, maintaining the temperature of the solution below −5° C. The resulting mixture was then left at −15° C. for 18 h. The formed solid was filtered off and washed with MeOH. The organic phase was dried and the solvent was evaporated in vacuo. The residue was purified by flash chromatography (silica gel, 1-5% methanol in dichloromethane) to give the title compound (9.70 g); ESMS m/z 249.9 [M+H]⁺.

### Intermediate 28

### 2-[(2-Hydroxymethyl-4-iodo-phenylamino)methylene]malonic Acid diethyl ester

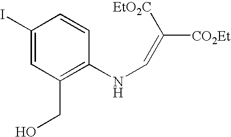

(2-Amino-5-iodo-phenyl)-methanol (9.70 g) and 2-ethoxymethylene-malonic acid diethyl ester (8.50 g) were mixed and heated at 110° C. in an open vessel under microwave heating for 20 min. After cooling the resulting solid was triturated with diethyl ether and filtered to give the title compound (9.78 g); ESMS m/z 419.8 [M+H]⁺.

### Intermediate 29

### 2-[(2-Acetoxymethyl-4-iodo-phenylamino)methylene]malonic Acid diethyl ester

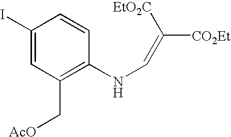

To a solution of 2-[(2-hydroxymethyl-4-iodo-phenylamino)methylene]malonic acid diethyl ester (9.78 g) and triethylamine (3.6 mL) in dry dichloromethane (100 mL) acetic anhydride (2.43 mL) was added at room temperature and the solution was stirred for 18 h. The solution was washed with water (2×50 mL) and with brine (50 mL). The organic phase was dried and the solvent was removed in vacuo to give the title compound (12.00 g) as a white solid; ESMS m/z 461.8 [M+H]⁺.

### Intermediate 30

### 8-Acetoxymethyl-6-iodo-4-oxo-1,4-dihydro-quinoline-3-carboxylic Acid ethyl ester

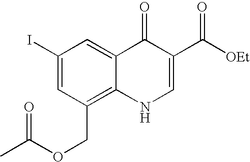

2-[(2-Acetoxymethyl-4-iodo-phenylamino)methylene]malonic acid diethyl ester (12.00 g) was dissolved in diphenylether (25 mL) and heated at 230° C. for 45 min under microwave heating in an open vessel. The crude was triturated with diisopropyl ether to give a first batch of the title product. A second batch was obtained by flash chromatography of the mother liquors (silica gel, 0-3% methanol in dichloromethane) to obtain the title product (total 4.50 g); ESMS m/z 415.9 [M+H]⁺.

### Intermediate 31

### 8-Hydroxymethyl-6-iodo-4-oxo-1,4-dihydro-quinoline-3-carboxylic Acid ethyl ester

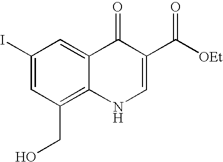

To a solution of 8-acetoxymethyl-6-iodo-4-oxo-1,4-dihydro-quinoline-3-carboxylic acid ethyl ester (4.50 g) in EtOH (60 mL) a solution of sodium ethoxide (0.330 g of sodium in 30 mL of EtOH) was added dropwise at 0° C. The resulting solution was stirred at room temperature for 1.5 h. The solution was concentrated in vacuo and the mixture was cooled at −15° C. for 18 h. The precipitated solid was filtered and washed with diisopropyl ether to give the title compound (2.86 g); ESMS m/z 373.9 [M+H]⁺.

### Intermediate 32

### 9-Iodo-3,3-dimethyl-7-oxo-1H,7H-[1,3]oxazino[5,4,3-ij]quinolone-6-carboxylic Acid ethyl ester

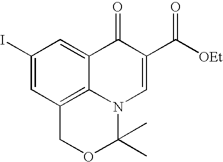

To a solution of 2,2-dimethoxypropane (12.4 mL) in dry DMF (12 mL) hydrated p-toluensulfonic acid (0.83 g) and molecular sieves 3A (1.7 g) were added under nitrogen. 8-hydroxymethyl-6-iodo-4-oxo-1,4-dihydro-quinoline-3-carboxylic acid ethyl ester (1.25 g) was added and the mixture was stirred at reflux under nitrogen for 1.5 h. The excess of 2,2-dimethoxypropane was evaporated, dichloromethane (15 mL) was added and the resulting solution was extracted with aqueous 5% NaHCO₃ (2×15 mL) and brine (15 mL). The organic phase was dried and the solvent was evaporated in vacuo. The residue was triturated with diisopropyl ether to give the title compound (1.03 g); ESMS m/z 413.8 [M+H]⁺.

### Intermediate 33

### Azithromycin 11,12-(N,N-dimethylformamide)acetal

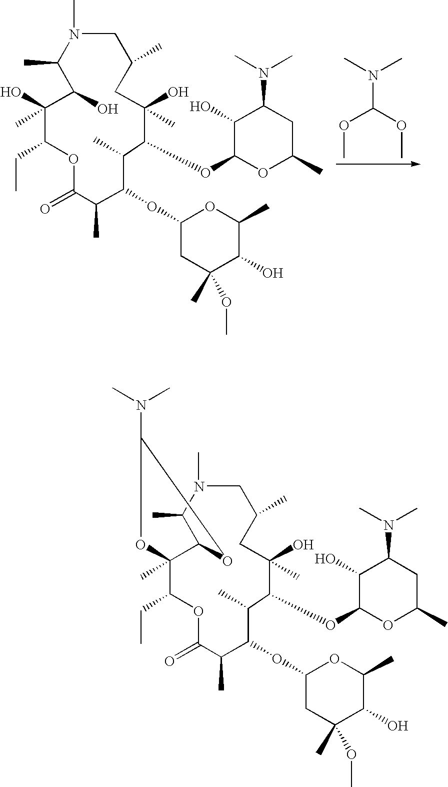

Azithromycin (50 g, 66.8 mmol) was dissolved in CHCl₃ (200 ml), N,N-dimethylformamide-dimethylacetal (28 ml, 211 mol) was added and reaction mixture stirred at the 65° C. for 24 hours. Then a further amount of N,N-dimethylformamide-dimethylacetal (14 ml, 105.5 mol) was added and the reaction mixture further stirred at the reflux temperature for 5 hours. The solvent was evaporated to give the crude title compound (50 g).

### Intermediate 34

### 2′-O-Acetyl-azithromycin 11,12-(N,N-dimethylformamide)acetal

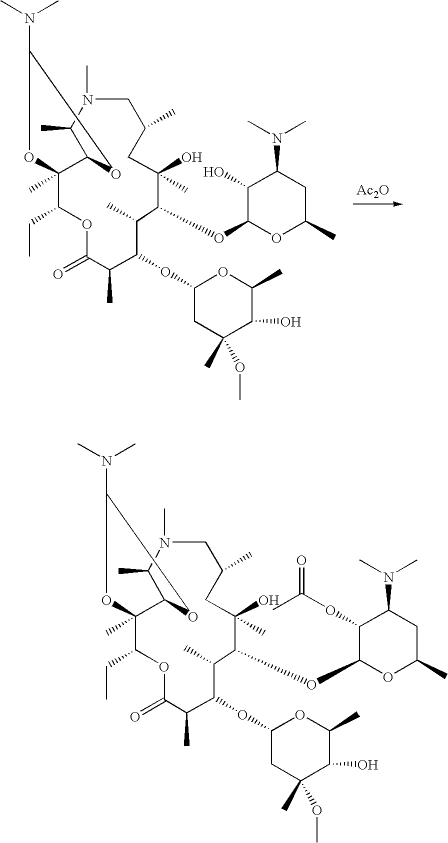

Azithromycin 11,12-(N,N-dimethylformamide)acetal (50 g, 62.2 mmol) was dissolved in EtOAc (500 ml), cooled to 0° C. and Ac₂O (8.8 ml, 94.5 mmol) was added dropwise. The reaction mixture was allowed to warm up to room temperature and stirred for 5 hours. Then, the reaction mixture was washed with saturated NaHCO₃ (2×150 ml), dried over K₂CO₃ and evaporated to dryness to give the crude title compound. The crude material was suspended in diethyleter (100 ml) at 0° C. for 20 minutes, filtered, and the solid washed with cold diethyleter (50 ml) and dried at 45° C. for 15 minutes to give the title product (52.68 g)

### Intermediate 35

### 2′-O-Acetyl-4″-O-(2-cyanoethyl)-azithromycin 11,12-(N,N-dimethylformamide)acetal

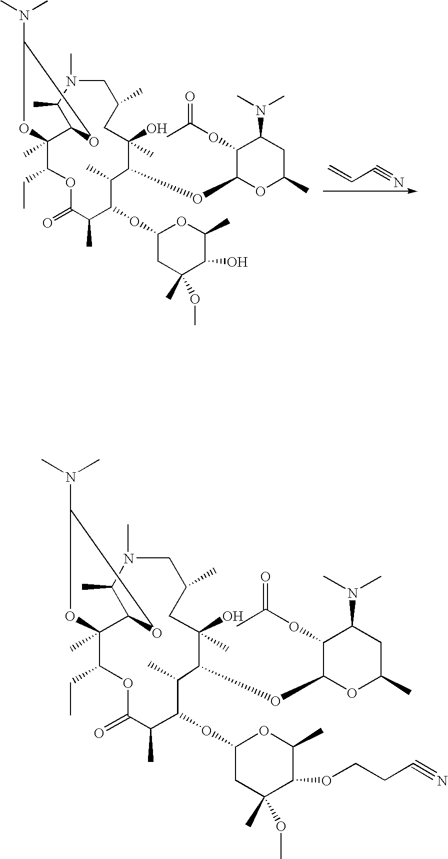

2′-O-Acetyl-azithromycin 11,12-(N,N-dimethylformamide)acetal (52.68 g, 62.3 mmol) was dissolved in acrylonitrile (350 ml, 5.32 mol) under N₂ flow. Then t-butanol (20 ml, 0.21 mol) was added and mixture cooled to 0° C., NaH 60% suspension in oil (2.9 g, 72.1 mmol) was added portionwise over 45 minutes. The reaction mixture was allowed to warm up to room temperature and stirred for additional 2 hours. Acrylonitrile was evaporated in vacuum, EtOAc (300 ml) was added, the resulting suspension was filtered through celite and the filtrate was washed with saturated NaHCO₃ (2×150 ml), dried over K₂CO₃ and evaporated to dryness to give the crude title compound. The crude material was dissolved in DCM (ca 50 ml), n-hexane was added (500 ml), the precipitate was filtered off and the filtrate evaporated to dryness to give the title product as a white solid (52.15 g).

### Intermediate 36

### 2′-O-Acetyl-4″-O-(3-aminopropyl)-azithromycin

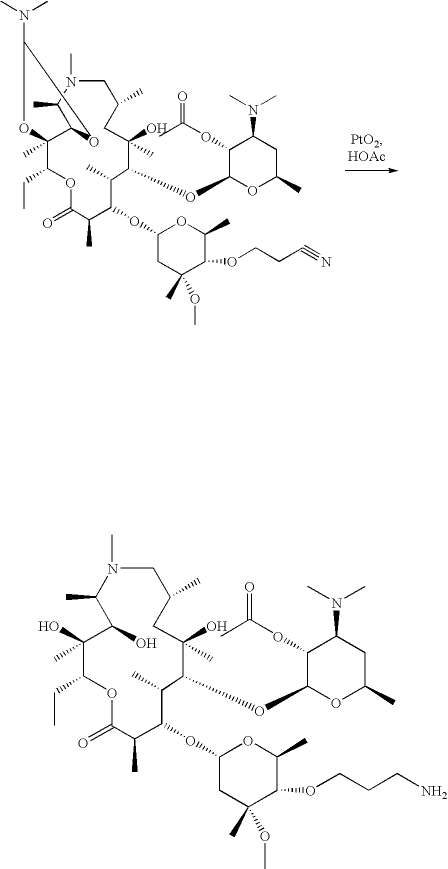

To a solution of 2′-O-acetyl-4″-O-(2-cyanoethyl)-azithromycin 11,12-(N,N-dimethylformamide)acetal (49.22 g, 54.7 mmol) in glacial acetic acid (150 ml) PtO₂ (5 g) was added and hydrogenated under H₂ pressure at 5 bar overnight. To the reaction mixture DCM (150 ml) was added, the resulting mixture filtered, and the filtrate evaporated to give an oily residue. Then DCM (150 m) and water (300 ml) were added and gradient extraction performed at pH 4.2, 6.5 and 8.15. Evaporation of organic layer at pH=8.15 gave the crude title product (25 g) which after suspending in diethyleter (40 ml) at 0° C., then filtered off to give the title product (19.19 g).

### Intermediate 37

### 4″-O-(3-Aminopropyl)-azithromycin

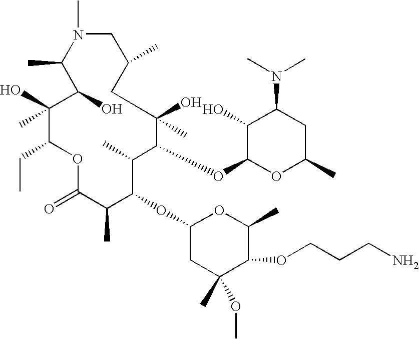

2′-O-Acetyl-4″-O-(3-aminopropyl)-azithromycin was deacetylated according to the procedure described for Intermediate 6 to obtain crude product (37.98 g). The crude material was suspended in diethyleter (50 ml) at 0° C., filtered off and dried in vacuum dryer to give the title product (33.81 g).

MS; m/z (ES): 806.5 [MH]⁺

### Alternative Preparation of Intermediate 37

### 4″-O-(3-Amino-propyl)-azithromycin

To a solution of Intermediate 2 (4 g, 4.6 mmol) in 2:1 MeOH/H₂O (150 ml), K₂CO₃ (6.3 g, 46 mmol) was added and the reaction mixture was stirred at 45° C. for 16 hours. After evaporation of MeOH, to the residue EtOAc (50 ml) was added and washed with saturated NaHCO₃ (2×50 ml). After evaporation of EtOAc the title product was obtained (3.6 g) as a white foamy residue.

MS; m/z (ES): 806.3 [MH]⁺

### Intermediate 38

### 4″-O-(3-But-3-ynyloxy-propyl)azithromycin

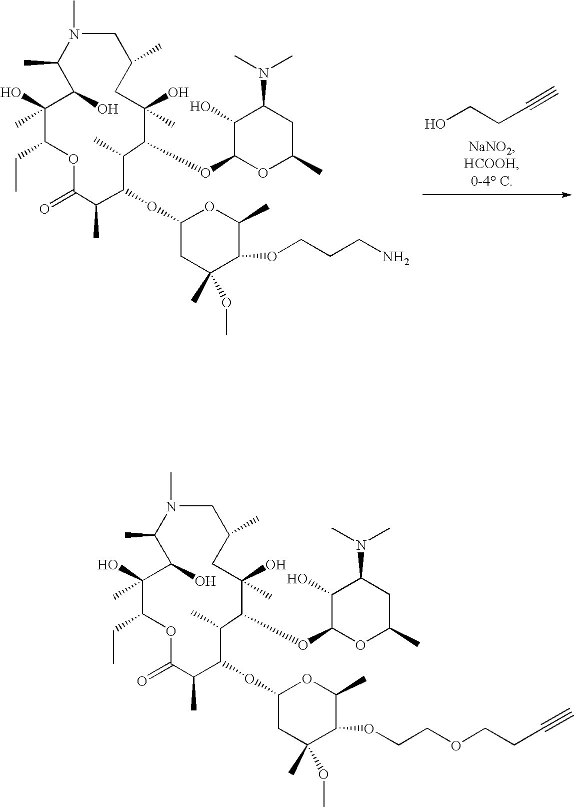

To a solution of 4″-O-(3-amino-propyl)-azithromycin (3.6 g, 4.5 mmol) in but-3-yn-1-ol (35 ml, 0.45 mol), HCOOH (0.77 ml, 20.4 mmol) was added under N₂ flow and cooled to −5° C. Then NaNO₂ (0.93 g, 13.5 mmol) was added portionwise over 45 minutes and the reaction mixture stirred at 0° C. for 5 hours and then at 4° C. overnight. Then an additional amount of NaNO₂ (0.155 g, 2.25 mmol) was added and stirred for further 3 hours at 4° C. The reaction mixture was diluted with EtOAc (30 ml) and washed with saturated NaHCO₃ (2×60 ml). To the organic layer water was added and pH adjusted to 3, the layers were separated, DCM (25 ml) was added to the aqueous layer, the pH adjusted to 6.3 and extracted with DCM (2×25 ml). To the combined organic layers (pH6.3) water was added, pH adjusted to 10, layers separated, organic one dried over K₂CO₃ and evaporated to dryness to give the foamy yellowish crude product (2.17 g) which was further purified by column chromatography (eluent DCM/MeOH:/NH₃=90:9:0.5) to give the title product (1.0 g).

MS; m/z (ES): 859.3 [MH]⁺

### Intermediate 39

### 4″-O-(3-Pent-4-ynyloxy-propyl)azithromycin

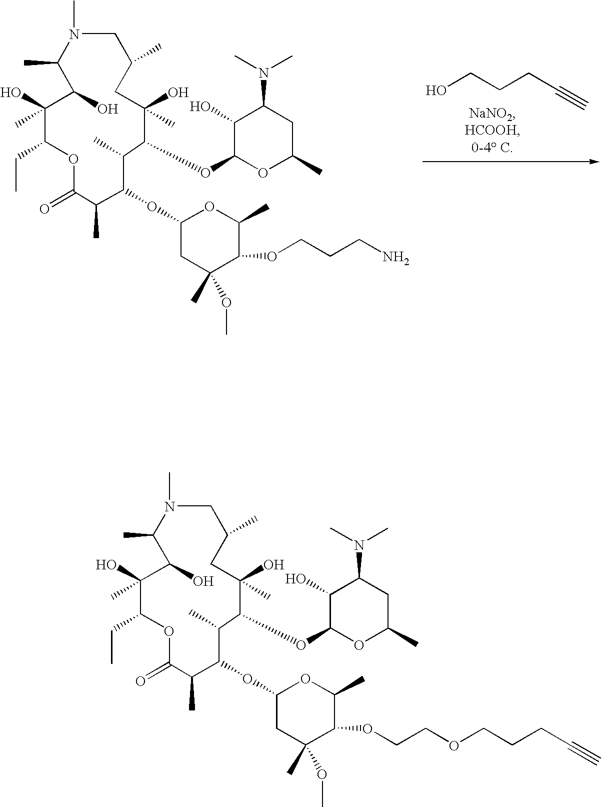

To a solution of 4″-O-(3-amino-propyl)-azithromycin (4.1 g, 5.11 mmol) in pent-4-yn-1-ol (47 ml, 0.51 mol), HCOOH (0.87 ml, 23 mmol) was added under N₂ flow and cooled to −5° C. Then NaNO₂ (1.2 g, 17.9 mmol) was added portionwise over 45 minutes and the reaction mixture stirred at 0° C. for 5 hours and then at 4° C. overnight. Then an additional amount of NaNO₂ (1.2 g, 17.9 mmol) was added and stirred at 4° C. overnight. After that the reaction mixture was warmed up to room temperature and stirred for further 6 hours. The reaction mixture was diluted with EtOAc (30 ml) and washed with saturated NaHCO₃ (2×60 ml). To the organic layer water was added and pH adjusted to 3, the layers were separated, DCM (25 ml) added to the aqueous layer, the pH adjusted to 6.3 and extracted with DCM (2×25 ml). To the combined pH 6.3 organic layers water was added, pH adjusted to 10, layers separated, organic one dried over K₂CO₃ and evaporated to dryness to give the oily brownish residue (4 g) which was further purified by column chromatography (eluent DCM/MeOH:/NH₃=90:9:0.5) to give the title product (1.15 g).

MS; m/z (ES): 873.4 [MH]⁺

### Intermediate 40

### Ethyl 6-iodo-1-[2-(methyloxy)ethyl]-4-oxo-1,4-dihydro-3-quinolinecarboxylate

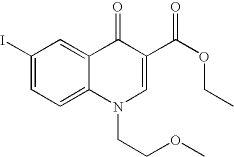

Ethyl 3-dimethylamino-2-(2-fluoro-5-iodobenzoyl)-2-propenoate (3.91 g) was suspended in ethanol (40 mL) at room temperature and treated with 2-aminoethylmethyl ether (0.825 g). After 0.5 h the homogeneous yellow solution formed was concentrated, dissolved in DMF (12 mL) and treated with potassium carbonate (2.07 g). The resultant mixture was heated at 70° C. for 3 h, cooled and allowed to stand at room temperature overnight. The solution was poured into water, the precipitated solid filtered. After washing with water the solid was dissolved in DCM, dried and evaporated to yield the title compound as a white solid (3.5 g); ESMS m/z 401.8 [M+H]⁺.

### Intermediate 41

### 6-Iodo-1-[2-(methyloxy)ethyl]-4-oxo-1,4-dihydro-3-quinolinecarboxylic acid

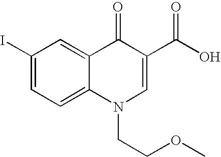

A mixture of ethyl 6-iodo-1-[2-(methyloxy)ethyl]-4-oxo-1,4-dihydro-3-quinolinecarboxylate (2.0 g) and 2M aqueous sodium hydroxide (2.64 mL) in THF (35 mL) was heated at 60° C. overnight. The THF was evaporated and the resultant mixture acidified (pH 6) with 10% aqueous citric acid solution. The solid precipitate thus formed was filtered and dried under vacuum over phosphorus pentoxide to yield the title compound (1.65 g).; ESMS m/z 373.8 [M+H]⁺.

### Intermediate 42

### 1-Dimethylamino-6-iodo-4-oxo-1,4-dihydro-3-quinolinecarboxylic acid

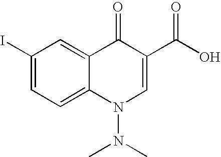

Ethyl 1-dimethylamino-6-iodo-4-oxo-1,4-dihydro-3-quinolinecarboxylate (3.09 g, 8 mmol) was dissolved in tetrahydrofuran (80 mL) then 2N aqueous sodium hydroxide (8.8 mL) was added and the reaction mixture stirred at 60° C. After 18 h tetrahydrofuran was removed, the resultant mixture acidified with 10% aqueous citric acid and the white precipitate filtered off, rinsed with water and dried under vacuum in the presence of diphosphorous pentoxide to give the title compound (2.86 g); ESMS m/z 358.9 [M+H]⁺.

### Intermediate 43

### Ethyl 1-(methoxy)-6-iodo-4-oxo-1,4-dihydroquinoline-3-carboxylate

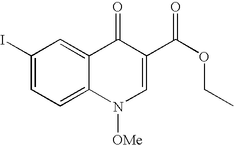

A mixture of ethyl 3-dimethylamino-2-(2-fluoro-5-iodobenzoyl)-2-propenoate (2.5 g), O-methylhydroxylamine hydrochloride (0.64 g) and potassium carbonate (2.21 g) in DMF (50 mL) was stirred at room temperature for 1 h then at 100° C. for 1 h and then cooled. The mixture was poured into water, the solid filtered off, then washed with water and dried The solid was purified by flash chromatography (silica gel, 0-5% MeOH in DCM) to give the title compound as a white solid (1.75 g); APCI m/z 374.0 [M+H]⁺.

### Intermediate 44

### 6-Iodo-1-(methyloxy)-4-oxo-1,4-dihydro-3-quinolinecarboxylic acid

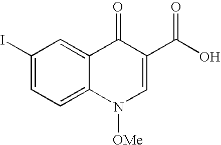

Ethyl 1-(methoxy)-6-iodo-4-oxo-1,4-dihydroquinoline-3-carboxylate (0.94 g) was suspended in THF (16 mL) and acetonitrile (5 mL) after heating gently to 30° C. with a hot air gun 0.5 M aqueous lithium hydroxide (10 mL) was added. The mixture was stirred for 4.5 h and acidified (pH 5) with 10% aqueous citric acid. The solid thus formed was filtered and dried under vacuum over phosphorus pentoxide to yield the title compound as a white solid (0.64 g); ESMS m/z 345.8 [M+H]⁺.

### Intermediate 45

### 4″-O-(2-Allyloxyethyl)-azithromycin

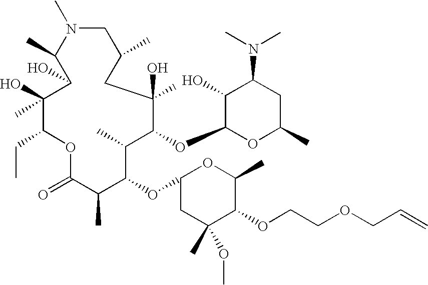

2′-O-Acetyl-4″-O-(2-allyloxyethyl)-azithromycin 11,12-carbonate (25.83 g, 28.7 mmol) was dissolved in methanol (500 ml) and treated with 10% aqueous potassium carbonate solution (250 ml). The reaction mixture was stirred at 55° C. for 2 h then allowed to cool down. Methanol was evaporated and the resulting material extracted three times with ethyl acetate. The combined organic phases were dried (K₂CO₃) then concentrated in vacuo. The resulting residue was purified by chromatography eluting with 0-10% (10:1 methanol/0.880 ammonia) in dichloromethane to give the title compound as a white solid (19.46 g); ESMS m/z 833.6 [M+H]⁺.

### Intermediate 46

### Ethyl 6-iodo-4-oxo-1-propyl-1,4-dihydro-3-quinolinecarboxylate

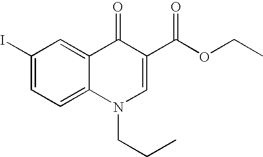

A solution of ethyl 3-dimethylamino-2-(2-fluoro-5-iodobenzoyl)-2-propenoate (4.5 g) in ethanol (40 mL) was treated with n-propylamine (1.04 mL). After stirring at 20° C. for 20 min the solvent was evaporated and the residue dissolved in DMF (50 mL) and treated with potassium carbonate (2.38 g). After stirring at 70° C. for 6 h the mixture was cooled and poured into water. The resultant precipitated solid was filtered, washed with water and dried to yield the title compound (1.54 g); ESMS m/z 385.9 [M+H]⁺.

### Intermediate 47

### 6-Iodo-4-oxo-1-propyl-1,4-dihydro-3-quinolinecarboxylic acid

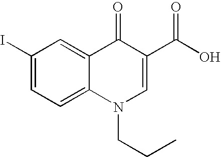

A mixture of ethyl 6-iodo-4-oxo-1-propyl-1,4-dihydro-3-quinolinecarboxylate (4.14 g) and 2M aqueous sodium hydroxide (5.6 mL) in THF (60 mL) was stirred and heated at 60° C. After 20 h the mixture was cooled and concentrated before acidifying (pH 5-6) with 10% aqueous citric acid. The resultant precipitated solid was filtered, washed with water and dried to yield the title compound as a white solid (3.55 g); ESMS m/z 357.9 [M+H]⁺.

### Intermediate 48

### Ethyl 6-iodo-1-methyl-4-oxo-1,4-dihydro-3-quinolinecarboxylate

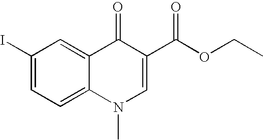

Ethyl 3-dimethylamino-2-(2-fluoro-5-iodobenzoyl)-2-propenoate (1.17 g) was suspended in ethanol (10 mL) at room temperature and treated with methylamine (0.35 mL). After 2 h a further portion of methylamine (0.05 mL) was added and the mixture stirred for 1 h. The solvent was evaporated and the residue dissolved in DMF (12 mL) and treated with potassium carbonate (0.62 g). After heating at 70° C. for 3 h, the reaction was cooled and allowed to stand at room temperature overnight. The solution was poured into water, the precipitated solid filtered and dried under vacuum over phosphorus pentoxide to yield the title compound (1.07 g); ESMS m/z 357.8 [M+H]⁺.

### Intermediate 49

### 6-Iodo-1-methyl-4-oxo-1,4-dihydro-3-quinolinecarboxylic acid

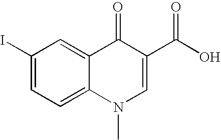

A mixture of ethyl 6-iodo-1-methyl-4-oxo-1,4-dihydro-3-quinolinecarboxylate (1.07 g) and 2M aqueous sodium hydroxide (3.15 mL) in THF (20 mL) was heated at 60° C. overnight. After this time the THF was evaporated and the mixture acidified (pH 5) by the addition of 10% aqueous citric acid. The resultant precipitate was filtered and dried under vacuum over phosphorus pentoxide to yield the title compound (0.91 g); ESMS m/z 329.8 [M+H]⁺.

### Intermediate 50

### Ethyl 6-iodo-1-(1-methylethyl)-4-oxo-1,4-dihydro-3-quinolinecarboxylate

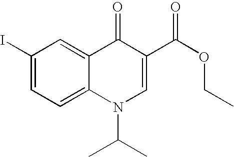

Ethyl 3-dimethylamino-2-(2-fluoro-5-iodobenzoyl)-2-propenoate (1.17 g) was suspended in ethanol (12 mL) at room temperature and treated with isopropylamine (0.279 mL). After 0.5 h the homogeneous yellow solution formed was concentrated, dissolved in DMF (12 mL) and treated with potassium carbonate (0.62 g). The resultant mixture was heated at 70° C. for 3 h, cooled and allowed to stand at room temperature overnight. The solution was poured into water and the precipitated solid filtered and dried under vacuum over phosphorus pentoxide to yield the title compound as a white solid (1.1 g); ESMS m/z 385.9 [M+H]⁺.

### Intermediate 51

### 6-Iodo-1-(1-methylethyl)-4-oxo-1,4-dihydro-3-quinolinecarboxylic acid

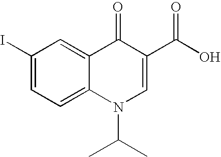

A mixture of ethyl 6-iodo-1-(1-methylethyl)-4-oxo-1,4-dihydro-3-quinolinecarboxylate (0.77 g) and 2M aqueous sodium hydroxide (1.05 mL) in THF (15 mL) was heated at 60° C. overnight. A further portion of 2M aqueous sodium hydroxide (1.05 mL) was added and the reaction heated for a further 3 h. After this time the mixture was cooled and acidified (pH 5) with 10% aqueous citric acid solution. The THF was evaporated and the solid precipitate filtered and dried under vacuum over phosphorus pentoxide to yield the title compound (0.6 g).; ESMS m/z 357.8 [M+H]⁺.

### Intermediate 52

### Ethyl 6-iodo-1-(1-cyclopropyl)-4-oxo-1,4-dihydro-3-quinolinecarboxylate

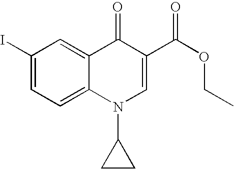

Ethyl 3-dimethylamino-2-(2-fluoro-5-iodobenzoyl)-2-propenoate (1.17 g) was suspended in ethanol (12 mL) at room temperature and treated with cyclopropylamine (0.229 mL). After 0.5 h the homogeneous yellow solution formed was concentrated, dissolved in DMF (12 mL) and treated with potassium carbonate (0.62 g). The resultant mixture was heated at 70° C. for 3 h, cooled and allowed to stand at room temperature overnight. The solution was poured into water and the precipitated solid filtered and dried under vacuum over phosphorus pentoxide to yield the title compound as a white solid (1.1 g); ESMS m/z [M+H]⁺.

### Intermediate 53

### 6-Iodo-1-(1-cyclopropyl)-4-oxo-1,4-dihydro-3-quinolinecarboxylic acid

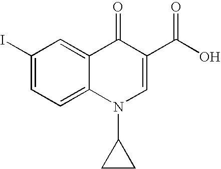

A mixture of ethyl 6-iodo-1-(1-cyclopropyl)-4-oxo-1,4-dihydro-3-quinolinecarboxylate (0.76 g) and 2M aqueous sodium hydroxide (1.05 mL) in THF (15 mL) was heated at 60° C. overnight. A further portion of 2M aqueous sodium hydroxide (1.05 mL) was then added and the reaction mixture heated for a further 3 h. After this time the mixture was cooled and acidified (pH 5) with 10% aqueous citric acid solution. The THF was evaporated and the solid precipitate filtered and dried under vacuum over phosphorus pentoxide to yield the title compound (0.7 g).; ESMS m/z 355.7 [M+H]⁺.

### Intermediate 54

### Diethyl 2-((3,4-dihydro-2H-quinolin-1-yl)methylene)malonate

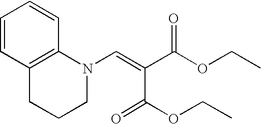

A mixture of tetrahydroquinoline (13.32 g) and diethyl ethoxymethylenemalonate (21.62 g) was heated to 130° C. using a Dean-Stark apparatus. After 1 h the reaction mixture was concentrated to give the title compound as a brown oil (30.4 g); ESMS m/z 304.3 [M+H]⁺.

### Intermediate 55

### Ethyl 1-oxo-6,7-dihydro-1H,5H-pyrido[3,2,1-ij]quinoline-2-carboxylate

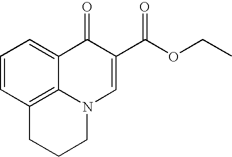

Diethyl 2-((3,4-dihydro-2H-quinolin-1-yl)methylene)malonate (2.5 g) was dissolved in polyphosphoric acid and the viscous mixture stirred for 4 h at 110° C. The reaction mixture was cooled down before adding ice. The resulting precipitate was filtered off, washed with water then dried in a dessicator in the presence of phosphorous pentoxide to give the title compound as a beige solid (0.815 g); ESMS m/z 258.2 [M+H]⁺.

### Intermediate 56

### Ethyl 9-bromo-1-oxo-6,7-dihydro-1H,5H-Pyrido[3.2.1-ij]quinoline-2-carboxylate

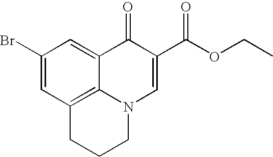

Ethyl 1-oxo-6,7-dihydro-1H,5H-pyrido[3,2,1-ij]quinoline-2-carboxylate (0.29 g) was dissolved in acetic acid (3 mL) and bromine (0.197 g) was added dropwise. The reaction was followed by LC/MS, additional bromine (2×0.197 g) was added. After 24 h water was added and the precipitate was filtered off, washed with Et₂O then dried in a dessicator in the presence of phosphorous pentoxide to provide an orange solid which was purified by flash chromatography (silica gel, 0-1.5% [9:1 MeOH:20M aqueous ammonia] in DCM) to give the title compound as a white solid (0.20 g);

ESMS m/z 336.1/338.1 [M+H]⁺.

### Intermediate 57

### 9-bromo-1-oxo-6,7-dihydro-1H,5H-pyrido[3,2,1-ij]quinoline-2-carboxylic acid

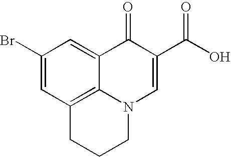

2N aqueous NaOH solution (470 mL, 0.92 mol, 1.1 eq) was added to a suspension of ethyl 9-bromo-1-oxo-6,7-dihydro-1H,5H-pyrido[3,2,1-ij]quinoline-2-carboxylate (286 g, 0.851 mol) in ethanol (2.5 L). After the addition was complete, the reaction mixture was heated at 90° C. for 3.5 h. LC/MS showed that the hydrolysis was complete, so the reaction mixture was cooled, the ethanol was removed under reduced pressure, and the resulting paste was acidified slowly with ice-cooled 3N aqueous HCl solution (˜400 mL). The resulting solid was collected by filtration, providing the title compound (236 g, 0.766 mol, 90% yield) as a light yellow solid after drying under vacuum. LCMS m/z 309 (MH⁺)

### Intermediate 58

### Ethyl 8-fluoro-1-(2-hydroxyethyl)-6-iodo-4-oxo-1,4-dihydro-3-quinoline carboxylate

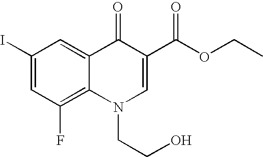

A suspension of potassium carbonate (4.14 g, 30 mmol), 2-iodoethanol (10 mL, 130 mmol) and ethyl 8-fluoro-6-iodo-4-oxo-1,4-dihydro-3-quinolinecarboxylate (Tucker et al WO 9932450) (3.61 g, 10 mmol) in DMF was heated at 100° C. After 2 h the mixture was cooled and the DMF evaporated. The crude product purified by chromatography over silica gel eluting with methanol 0 to 10% in dichloromethane to give the title compound (2.6 g); ESMS m/z 406.1 [M+H]⁺.

### Intermediate 59

### Ethyl 9-iodo-7-oxo-2,3-dihydro-7H-[1,4]oxazino[2,3,4-ij]quinoline-6-carboxylate

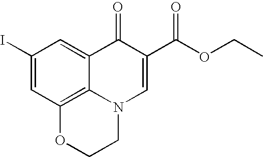

Ethyl 8-fluoro-1-(2-hydroxyethyl)-6-iodo-4-oxo-1,4-dihydro-3-quinoline carboxylate (0.810 g, 2 mmol) was dissolved in DMF (20 mL) and treated with diazabicycloundecane (0.3 mL, 2 mmol). After 17 h at 100° C. the reaction mixture was concentrated and the mixture purified by chromatography over silica gel eluting with 0 to 5% [9:1 methanol/20M ammonium hydroxide] in dichloromethane to give the title compound (0.77 g); ESMS m/z 386.1 [M+H]⁺.

### Intermediate 60

### 9-Iodo-7-oxo-2,3-dihydro-7H-[1,4]oxazino[2,3,4-ij]quinoline-6-carboxylic acid

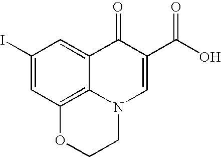

Ethyl 9-iodo-7-oxo-2,3-dihydro-7H-[1,4]oxazino[2,3,4-ij]quinoline-6-carboxylate (0.24 g) was suspended in THF (10 mL) and treated with 2M aqueous sodium hydroxide (0.65 mL). After stirring overnight at 60° C. the mixture was cooled and the THF evaporated. The resultant mixture was acidified (pH 5) with 10% aqueous citric acid and the precipitated solid filtered, dried under vacuum to yield a mixture of 85% acid and 15% ester. This material was re-suspended in THF/MeOH and treated with 2M aqueous sodium hydroxide (0.65 mL). After heating at 70° C. overnight the above work-up was repeated to yield the title compound (0.19 g). ESMS m/z [M+H]⁺.

### Intermediate 31—Additional Preparation

### 8-Hydroxymethyl-6-iodo-4-oxo-1,4-dihydro-quinoline-3-carboxylic acid ethyl ester

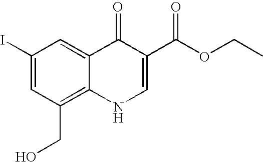

A suspension of 8-acetoxymethyl-6-iodo-4-oxo-1,4-dihydro-quinoline-3-carboxylic acid ethyl ester (1 g, 2.4 mmol) and sodium ethoxide (0.165 g, 2.4 mmol) in ethanol (50 mL) was heated at 80° C. for 2 h. The reaction mixture was allowed to cool down then the product was pre-absorbed on silica gel and purified by chromatography eluting with 0 to 25% [9:1 methanol/20M ammonia] in dichloromethane to give the title compound. (0.73 g) as a beige solid; ESMS m/z 374.0 [M+H]⁺.

### Intermediate 61

### Methyl 9-iodo-3,3-dimethyl-7-oxo-1H,7H-[1,3]oxazino[5,4,3-ij]quinoline-6 carboxylate

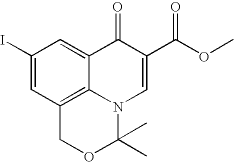

A suspension of ethyl 8-hydroxymethyl-6-iodo-4-oxo-1,4-dihydro-quinoline-3-carboxylate (24.71 g), p-toluene sulfonic acid monohydrate (10.45 g) and 2,2-dimethoxypropane (81 mL) in N-methylpyrrolidinone (57 mL) was heated at 80° C. under argon for 3 h. Additional quantities of 2,2-dimethoxypropane were added after 5, 8 and 16 h. The reaction mixture was then concentrated and the residue freeze dried overnight. The resultant solid was treated with water and subjected to sonication, the water decanted and disgarded. The solid was then sonicated with DCM, the residual solid was mostly starting material (8.5 g). The DCM layer contained the methyl ester. This was purified by chromatography over silica gel eluting with 0-10% (10:1 methanol/0.880 ammonia) in dichloromethane to give the title compound as a light brown solid (15.31 g) ESMS m/z 400.1 [M+H]⁺.

### Intermediate 62

### 9-Iodo-3,3-dimethyl-7-oxo-1H,7H-[1,3]oxazino[5,4,3-ij]quinoline-6-carboxylic Acid

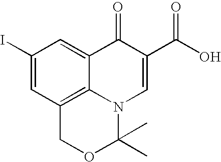

Methyl 9-iodo-3,3-dimethyl-7-oxo-1H,7H-[1,3]oxazino[5,4,3-ij]quinoline-6 carboxylate (0.789 g) suspended in THF (14 mL) and aqueous sodium hydroxide (1.1 mL) was heated at 60° C. overnight. After this time the mixture was cooled, the THF evaporated and the residue diluted with water. Acidification (pH 5) by the addition of 10% aqueous citric acid resulted in the precipitation of a solid which was filtered and dried under vacuum over phosphorus pentoxide overnight to yield the title compound as a light pink solid (6.66 g);

ESMS m/z 385.8 [M+H]⁺.

### Intermediate 63

### Ethyl 1-(cyclopropylmethyl)-6-iodo-4-oxo-1,4-dihydro-3-quinolinecarboxylate

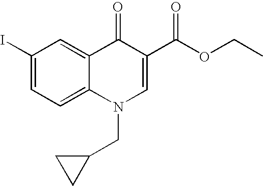

To a stirred suspension of ethyl 6-iodo-4-oxo-1,4-dihydro-3-quinolinecarboxylate (7.5 g) and potassium carbonate (6.0 g) in DMF at 50° C. was added cyclopropylmethyl bromide (5.9 g). After 1.5 h a further portion of cyclopropylmethyl bromide (2 mL) was added and the heating/stirring continued overnight. The mixture was then cooled to room temperature and the DMF evaporated. The residue was partitioned between water and DCM, the organic layer separated, dried and evaporated to yield the title compound as a pale yellow solid (6.5 g). ESMS m/z 397.8 [MH]⁺.

### Intermediate 64

### 1-(Cyclopropylmethyl)-6-iodo-4-oxo-1,4-dihydro-3-quinolinecarboxylic Acid

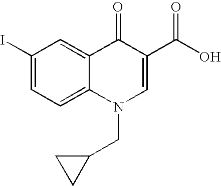

To a stirred solution of ethyl 1-(cyclopropylmethyl)-6-iodo-4-oxo-1,4-dihydro-3-quinolinecarboxylate (6.5 g) in THF (100 mL) at room temperature was added 2M aqueous sodium hydroxide (40 mL). After 2 h 40% w/v aqueous sodium hydroxide (40 mL) and methanol (40 mL) were added and the reaction allowed to continue for another 16 h. The mixture was made acidic by the addition of concentrated hydrochloric acid and the resultant precipitated material extracted into DCM (200 mL). The organic layer was dried, evaporated and left under vacuum (4 mbar) at 50° C. to yield the title compound as a beige solid (5.0 g). ESMS m/z 369.8 [MH]⁺.

### Intermediate 65

### Ethyl 1-butyl-6-iodo-4-oxo-1,4-dihydro-3-quinolinecarboxylate

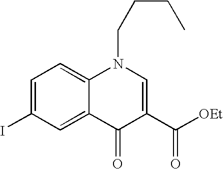

Ethyl 6-iodo-4-oxo-1,4-dihydro-3-quinolinecarboxylate (8.62 g, 25.1 mmol) was dissolved in DMF (100 mL), then treated with potassium carbonate (6.95 g, 50.3 mmol) and iodobutane (13.88 g, 75.4 mmol). The resulting mixture was stirred under argon at 65° C. for 20 h, allowed to cool and then concentrated to ⅓ volume. The residue was poured into water and ultrasonicated, then the precipitated solid was filtered off, washed with water and dried. This solid was purified by silica gel chromatography eluting with 0-4% (10:1 methanol/0.880 ammonia) in dichloromethane to give the title compound as a white solid (2.80 g); ESMS m/z 399.8 [M+H]⁺.

### Intermediate 66

### Ethyl 1-ethyl-6-allyl-4-oxo-1,4-dihydro-3-quinolinecarboxylate

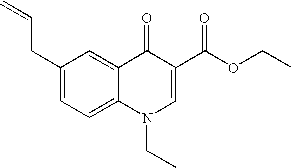

Tetrabutylammonium acetate (3.77 g, 12.5 mmol), was dissolved in dimethylformamide (20 mL) then 4 Å molecular sieves (0.8 g) were added and the mixture stirred at room temperature under argon for 1.5 h. Bis(dibenzylideneacetone) palladium (0.115 g, 0.2 mmol) and triphenylphosphine (0.21 g, 0.8 mmol) were added followed by ethyl 1-ethyl-6-iodo-4-oxo-1,4-dihydro-3-quinolinecarboxylate (1.855 g, 5 mmol) then allyltrimethylsilane (4 mL, 25 mmol). The reaction mixture was stirred at 50° C. under argon. After 18 h dimethylformamide was removed in vacuo and the resultant mixture diluted with ethyl acetate, filtered and diluted with additional ethyl acetate. The filtrate was washed with water, the organic layer was decanted off and the remainder extracted three times with dichloromethane. The combined organic layers were dried, the solvent evaporated and the residue purified by chromatography on silica gel eluting with 100% hexane to 100% ethyl acetate to give the title compound (1.035 g); ESMS m/z 286.1 [M+H]⁺.

### Intermediate 67

### 1-Ethyl-6-allyl-4-oxo-1,4-dihydro-3-quinolinecarboxylic acid

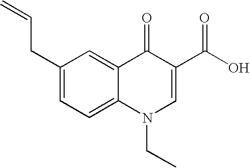

Ethyl 1-ethyl-6-allyl-4-oxo-1,4-dihydro-3-quinolinecarboxylate (0.285 g, 1 mmol) was dissolved in tetrahydrofuran (10 mL) then 2N aqueous sodium hydroxide (1.1 mL) was added and the reaction mixture stirred at 60° C. After 17 h tetrahydrofuran was removed in vacuo, the resultant mixture acidified with 10% aqueous citric acid and the white precipitate filtered off and dried under vacuum in the presence of diphosphorous pentoxide over 17 h to give the title compound (0.257 g); ESMS m/z 257.9 [M+H]⁺.

### Intermediate 68

### 1-Ethyl-6-iodo-4-oxo-1,4-dihydro-3-quinolinecarboxylic acid

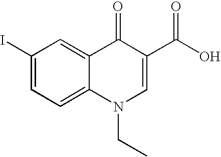

Ethyl 1-ethyl-6-iodo-4-oxo-1,4-dihydro-3-quinolinecarboxylate (7.42 g, 20 mmol) was dissolved in tetrahydrofuran (140 mL) then 2N aqueous sodium hydroxide (10.56 mL) was added and the reaction mixture stirred at 60° C. After 18 h tetrahydrofuran was removed, the resultant mixture diluted with water, acidified with 10% aqueous citric acid and the white precipitate filtered off and dried under vacuum in the presence of diphosphorous pentoxide to give the title compound (6.6 g); ESMS m/z 343.7 [M+H]⁺.

### Intermediate 69

### Ethyl 3-(2-dimethylamino-ethylamino)-2-(2-fluoro-5-Iodo-benzoyl)-2-propenoate

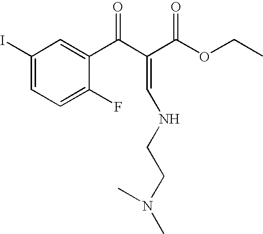

A suspension of ethyl 3-dimethylamino-2-(2-fluoro-5-iodo-benzoyl)-2-propenoate (23 g, 0.059 mol) in diethyl ether/EtOH=2:1 (132 ml) was cooled to 0° C. and N,N-dimethylethylendiamine (12.9 ml, 0.12 mol, 2 eq.) was added dropwise. Stirring was continued at 0° C. for 3 hours. The solvents were evaporated in vacuum. The product was precipitated by adding n-hexane, filtered off yielding ethyl 3-(2-dimethylamino-ethylamino)-2-(2-fluoro-5-iodo-benzoyl)-2-propenoate (18.68 g).

MS; m/z (ES): 434.2 [MH]+

### Intermediate 70

### Ethyl 1-(2-Dimethylamino-ethyl)-6-iodo-4-oxo-1,4-dihydro-quinoline-3-carboxylate

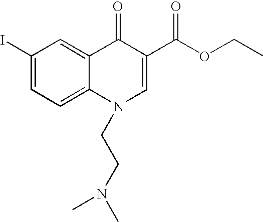

To a solution of ethyl 3-(2-dimethylamino-ethylamino)-2-(2-fluoro-5-iodo-benzoyl)-2-propenoate (18.68 g, 0.043 mol) in DMF (112 ml), K₂CO₃ (11.89 g, 0.086 mol, 2 eq.) was added and the reaction mixture was stirred at 140° C. for 4 hours, filtered and the filtrate evaporated in vacuum. The product was precipitated by adding EtOAc, and filtered off yielding 5.69 g of ethyl 1-(2-dimethylamino-ethyl)-6-iodo-4-oxo-1,4-dihydro-quinoline-3-carboxylate.

MS; m/z (ES): 415.0 [MH]⁺

### Intermediate 71

### 1-(2-Dimethylamino-ethyl)-6-iodo-4-oxo-1,4-dihydro-quinoline-3-carboxylic Acid

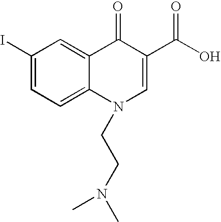

To a solution ethyl of 1-(2-dimethylamino-ethyl)-6-iodo-4-oxo-1,4-dihydro-quinoline-3-carboxylate (1.5 g, 3.6 mmol) in THF (20 ml), a solution of NaOH (0.68 g, 17 mmol, 4.8 eq.) in water (20 ml) was added and the reaction mixture was stirred at 70° C. for 1 hour. THF was evaporated in vacuum and pH of the aqueous layer was adjusted to 2.5. The product precipitated, was filtered off and dried at 80° C. yielding 1.18 g of 1-(2-dimethylamino-ethyl)-6-iodo-4-oxo-1,4-dihydro-quinoline-3-carboxylic acid.

MS; m/z (ES): 386.9 [MH]⁺

### Intermediate 72

### Ethyl 3-tert-butylamino-2-(2-fluoro-5-iodo-benzoyl)-2-propenoate

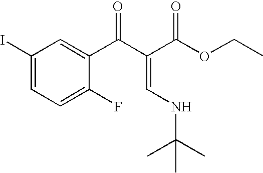

To a suspension of ethyl 3-dimethylamino-2-(2-fluoro-5-iodo-benzoyl)-2-propenoate (10.24 g, 0.026 mol) in diethyl ether/EtOH=2:1, (60 ml) tert-butylamine (5.45 ml, 0.052 mol, 2 eq.) was added dropwise. The reaction mixture was stirred at room temperature for 2 hours. The solvents were evaporated in vacuum. The product was precipitated by adding n-hexane, and filtered off yielding 5.37 g of ethyl 3-tert-butylamino-2-(2-fluoro-5-iodo-benzoyl)-2-propenoate.

MS; m/z (ES): 419.2 [MH]⁺

### Intermediate 73

### Ethyl 1-tert-Butyl-6-iodo-4-oxo-1,4-dihydro-quinoline-3-carboxylate

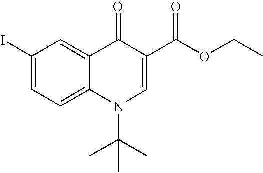

To a solution of ethyl 3-tert-butylamino-2-(2-fluoro-5-iodo-benzoyl)-2-propenoate (5.37 g, 0.013 mol) in DMF (32 ml), K₂CO₃ (3.54 g, 0.026 mol, 2 eq.) was added and the reaction mixture was stirred at 140° C. for 4 hours and then filtered. The product was left to precipitate form the filtrate and was filtered off yielding ethyl 1-tert-butyl-6-iodo-4-oxo-1,4-dihydro-quinoline-3-carboxylate (3.18 g).

MS; m/z (ES): 399.9 [MH]⁺

### Intermediate 74

### 1-tert-Butyl-6-iodo-4-oxo-1,4-dihydro-quinoline-3-carboxylic acid

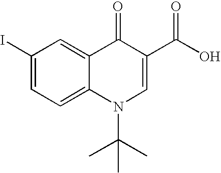

To a solution of ethyl 1-tert-butyl-6-iodo-4-oxo-1,4-dihydro-quinoline-3-carboxylate (1.5 g, 3.8 mmol) in THF (20 ml), a solution of NaOH (0.72 g, 18 mmol, 4.8 eq.) in water (20 ml) was added and the reaction mixture was stirred at 70° C. for 1 hour. THF was evaporated in vacuum and pH of the aqueous layer was adjusted to 2.5. The product precipitated, was filtered off and dried at 80° C. yielding 1-tert-butyl-6-iodo-4-oxo-1,4-dihydro-quinoline-3-carboxylic acid (1.26 g).

MS; m/z (ES): 371.9 [MH]⁺

### Example 1

### 4″-O-{3-[3-(3-Carboxy-1-dimethylamino-4-oxo-1,4-dihydro-quinolin-6-yl)-prop-2-en-1-yloxy]-propyl}-azithromycin

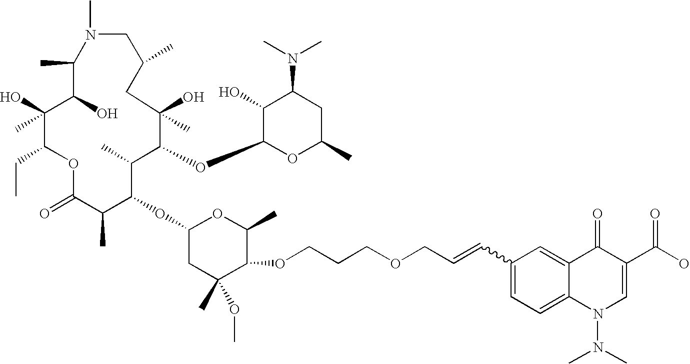

To a solution of Intermediate 6 (0.26 g, 0.31 mmol) in DMF (5 ml), Pd(OAc)₂ (0.014 g, 0.062 mmol) and TOTF (0.032 g, 0.124 mmol) were added under N₂ flow. After 1 hour 1-dimethylamino-6-iodo-4-oxo-1,4-dihydro-quinoline-3-carboxylic acid (0.28 g, 0.78 mmol) and Et₃N (0.17 ml, 1.24 mmol) were added, N₂ flow was removed and the reaction mixture stirred at 65° C. for 2 hours and then at 75° C. for further 17 hours. After catalysts were filtered off, EtOAc (20 ml) was added and the reaction mixture washed with aqueous NaHCO₃ (3×20 ml). The organic layer was dried over K₂CO₃ and evaporated in vacuum. Precipitation from EtOAc/n-hexane yielded the title compound (350 mg).

MS; m/z (ES): 1077.6 [MH]⁺

### Example 2

### 4″-O-{3-[3-(3-Carboxy-1-dimethylamino-4-oxo-1,4-dihydro-quinolin-6-yl)-prop-2-ynyloxy]-propyl}-azithromycin

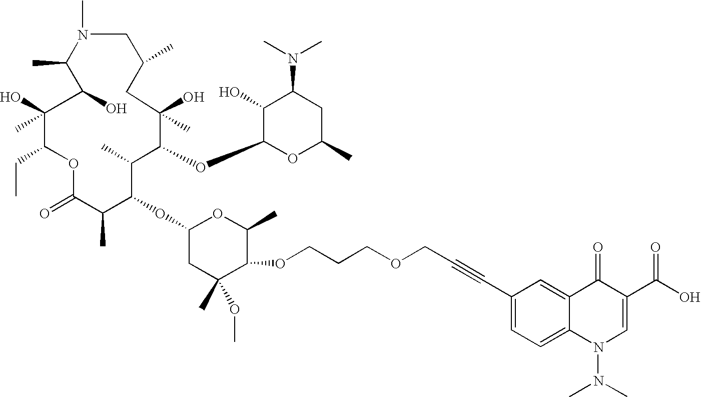

To a suspension of 1-dimethylamino-6-iodo-4-oxo-1,4-dihydro-quinoline-3-carboxylic acid (3 g, 8.4 mmol) in MeCN (20 ml), CuI (180 mg, 1 mmol) and Et₃N (6.7 ml, 48 mmol) were added and the reaction mixture stirred at room temperature for 20 minutes under N₂ flow and then heated to 50° C. Solution of Intermediate 8 (4 g, 4.8 mmol) in MeCN (15 ml) and Pd(PPh₃)₂Cl₂ (167 mg, 0.24 mmol) were added and stirring was continued for 3 hours. Than an additional amount of 1-dimethylamino-6-iodo-4-oxo-1,4-dihydro-quinoline-3-carboxylic acid (1 g, 2.8 mmol) was added and stirring continued overnight. A second batch of material was prepared in a same manner and combined with the first batch. The combined reaction mixtures were filtered and MeCN evaporated under reduced pressure. EtOAc (100 ml) was added to the residue, and the precipitate formed was removed by filtration. The filtrate was washed with saturated aqueous NaHCO₃ (2×100 ml, 1×50 ml) followed by extraction with water (100 ml) at pH 3.4. Then the water layer was extracted with EtOAc (150 ml) at pH 8.5. The organic layer at pH 8.5 was dried over K₂CO₃ and evaporated under reduced pressure. The crude product was precipitated from EtOAc/n-hexane (15 ml:150 ml), filtered off, dryed at 40° C. for 2 hours yielding the title compound after filtration (5.5 g).

MS (ES+) m/z: [MH]⁺=1075.6.

### Example 3

### 4″-O-{3-[3-(3-Carboxy-1-dimethylamino-4-oxo-1,4-dihydro-quinolin-6-yl)-propoxy]-propyl}-azithromycin

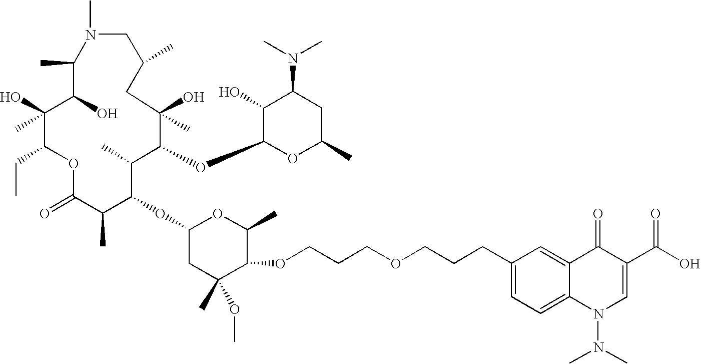

### Method A

The compound of Example 1 (0.35 g, 0.32 mmol) was dissolved in MeOH (30 ml), catalyst 10% Pd/C (0.18 g) was added and hydrogenation performed over 7 hours at 5 bar H₂ pressure. The catalyst was filtered off, solvent evaporated under reduced pressure. The crude product was first purified by column chromatography (first using solvent system: DCM/MeOH/NH₃=90:9:1.5 and then DCM/MeOH/NH₃=90:15:1.5) and then precipitated from EtOAc/n-hexane yielding the title compound (0.1 g).

MS (ES+) m/z: [MH]⁺=1079.3

### Method B

The compound of Example 2 (5.5 g, 1.5 mmol) was dissolved in MeOH (100 ml), catalyst 10% Pd/C (1.3 g) was added and hydrogenation performed over 15 hours at 2.2 bar H₂ pressure. The catalyst was filtered off, and the solvent evaporated in vacuum. The crude product (4.8 g) was first purified by column chromatography (using solvent system: DCM/MeOH/NH₃=90:9:0.5) to give the title product (0.610 g, purity 91% LC/MS) which may be optionally purified by following procedure. The product (0.610 g) was dissolving in EtOAc (30 ml), water (30 ml) added and pH adjusted to 3. Organic layer was discharged, to the water one DCM (30 ml) was added and pH adjusted to 4. Organic layer (pH 4) was discharged, to the water layer DCM (40 ml) was added and pH adjusted to pH 4.5. Water layer was extracted once more with DCM (30 ml). To the combined organic layers at pH 4.5 water (50 ml) was added, pH adjusted to 10.7, layers separated, organic layer (pH 10.7) evaporated and residue precipitated form EtOAc/n-hexane to give a white solid which after drying at 40° C. for 90 minutes gave the title product (380 mg, purity 93% LC/MS).

MS (ES+) m/z: [MH]⁺=1079.6

¹³C-NMR (125 MHz. CDCl₃)/δ: 178.54 (C-1), 177.58 (Q-p), 166.43 (Q-o), 141.56 (Q-h), 140.82 (Q-u), 138.56 (Q-s), 134.89 (Q-t), 126.53 (Q-r), 126.21 (Q-f), 116.35 (Q-g), 108.83 (Q-k), 101.84 (C-1′), 94.51 (C-1″), 87.36 (C-4″), 82.94 (C-5), 77.45 (C-3+C-13), 73.90 (C-6), 73.51 (C-12), 73.37 (C-11), 73.29 (C-3″), 71.12 (CH₂), 70.68 (C-2′), 69.68 (C-9), 69.26 (CH₂), 67.42 (C-5′), 67.37 (CH₂), 64.99 (C-3′), 64.41 (C-5″), 62.09 (C-10), 49.19 (3″OMe), 45.87 (N(CH₃)₂), 44.87 (C-2), 41.95 (C-7), 41.65 (C-4), 40.02 (3′NMe₂), 35.95 (9aNMe), 35.05 (C-2″), 31.62 (CH₂), 30.78 (CH₂), 30.29 (CH₂), 4′ (n.d.), 27.09 (6Me), 26.40 (C-8), 21.61 (8Me), 21.35 (3″Me+5′Me), 20.91 (C-14), 18.08 (5″Me), 15.85 (12Me), 14.38 (2Me), 10.89 (C-15), 8.86 (4Me), 7.08 (10Me).

### Example 4

### 4″-O-{3-[3-(3-Carboxy-1-cyclopropyl-4-oxo-1,4-dihydro-quinolin-6-yl)-prop-2-en-1-yloxy]-propyl}-azithromycin

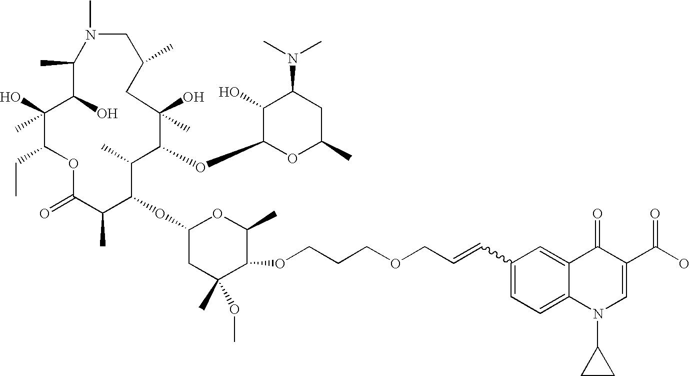

Intermediate 6 (320 mg, 0.38 mmol, 1 equiv.) in DMF (5 ml), Pd(OAc)₂ (0.017 g, 0.08 mmol) and TOTF (0.046 g, 0.15 mmol) were added and stirred under N₂ flow for 1 hour. Than 1-cyclopropyl-6-iodo-4-oxo-1,4-dihydro-quinoline-3-carboxylic acid (400 mg, 1.1 mmol, 2.5 equiv.), Et₃N (0.21 ml, 1.52 mmol) were added, N₂ flow was removed and the reaction mixture stirred at 65° C. for 2 hours and then at 75° C. overnight. After catalysts were filtered off, EtOAc (70 ml) was added and the reaction mixture washed with aqueous NaHCO₃ (2×100 ml). To the organic layer water (50 ml) was added, pH adjusted to 3 and organic layer discharged. To the water layer EtOAc (50 ml) was added, pH adjusted to 8.5 and layers separated. The organic layer (pH 8.5) was dried over K₂CO₃ and evaporated and obtained residue precipitated from EtOAc/n-hexane yielding the title compound (357 mg).

MS (ES+) m/z: [MH]⁺=1074.6

¹³C-NMR (125 MHz. CDCl₃) δ: 179.17 (C-1), 178.63 (Q-p), 167.20 (Q-o), 147.66 (Q-h), 146.40 (CH═CH), 140.69 (Q-s), 139.45 (Q-u), 134.74 (Q-t), 125.98 (Q-r), 125.51 (Q-f), 117.25 (Q-g), 108.52 (Q-k), 103.82 (CH═CH), 102.43 (C-1′), 87.84 (C-4″), 83.08 (C-5), 77.71 (C-3+C-13), 74.21 (C-12), 73.84 (C-3″), 73.73 (C-6), 73.57 (C-11), 71.00 (C-2′), 70.62 (CH₂), 70.09 (C-9), 68.94 (CH₂), 67.91 (C-5′), 65.36 (C-3′), 64.69 (C-5″), 62.53 (C-10), 49.64 (3″OMe), 45.36 (C-2), 42.37 (C-4), 42.24 (C-7), 40.30 (3′NMe₂), 36.19 (9aNMe), 35.41 (Q-i), 35.40 (C-2″), 30.61 (CH₂), 29.68 (CH₂), 28.89 (C-4′), 27.58 (6Me), 26.79 (C-8), 21.98 (8Me), 21.82 (5′Me), 21.67 (3″Me), 21.30 (C-14), 18.30 (5″Me), 16.24 (12Me), 14.59 (2Me), 11.26 (C-15), 9.02 (4Me), 8.25 (Q-j), 7.28 (10Me)

### Example 5

### 4″-O-{3-[3-(3-Carboxy-1-ethyl-4-oxo-1,4-dihydro-quinolin-6-yl)-prop-2-en-1-yloxy]-propyl}-azithromycin

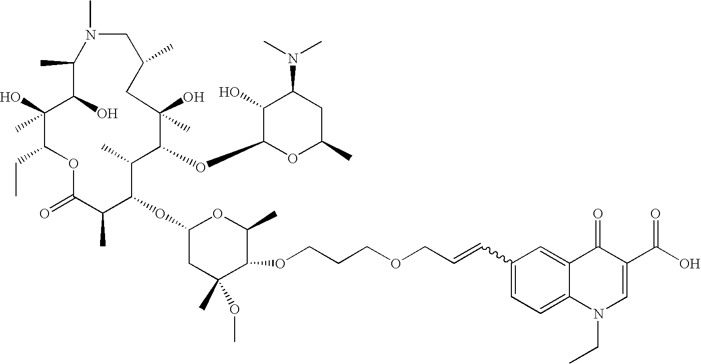

Starting from Intermediate 6 (400 mg, 1 equiv.) and 1-ethyl-6-iodo-4-oxo-1,4-dihydro-quinoline-3-carboxylic acid (400 mg, 2.5 equiv.) according to the procedure of Example 4 (except that the reagents were pre-mixed under N₂ flow for 2.5 hours, then the mixture was stirred for 5 hours at 65° C. and 16 hours at 75° C.) the title compound was obtained (355 mg).

MS (ES+) m/z: [MH]⁺=1062.7

¹³C-NMR (125 MHz. CDCl₃) δ: 179.25 (C-1), 178.63 (Q-p), 147.54 (Q-h), 146.15 (CH═CH), 137.42 (Q-u), 134.28 (Q-t), 125.98 (Q-r), 126.12 (Q-f), 116.09 (Q-g), 108.52 (Q-k), 103.99 (CH═CH), 102.58 (C-1′), 94.73 (C-1″), 87.73 (C-4″), 83.08 (C-5), 77.48 (C-3+C-13), 74.35 (C-12), 74.12 (C-11), 73.84 (C-6), 83.74 (C-3″), 70.97 (C-2′), 70.56 (CH₂), 70.09 (CH₂), 68.72 (CH₂), 68.04 (C-5′), 65.38 (C-3′), 64.76 (C-5″), 62.49 (C-10), 49.72 (3″OMe), 49.38 (CH₂), 45.38 (C-2), 42.37 (C-7), 42.31 (C-4), 40.34 (3′NMe₂), 36.32 (9aNMe), 35.33 (C-2″), 27.8 (6Me), 26.78 (C-8), 22.03 (8Me), 21.84 (3″Me), 21.60 (5′Me), 21.28 (C-14), 18.44 (5″Me), 16.48 (12Me), 14.62 (2Me+CH₃), 11.21 (C-15), 9.09 (4Me), 7.59 (10Me).

### Example 6

### 4″-O-{3-[3-(3-Carboxy-1-methoxyeth-2-yl-4-oxo-1,4-dihydro-quinolin-6-yl)-prop-2-en-1-yloxy]-propyl}-azithromycin

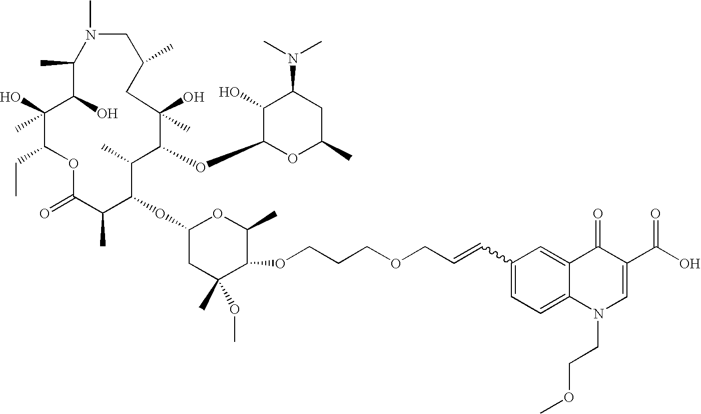

Starting from Intermediate 6 (400 mg, 1 equiv.) and 1-methoxyethyl-6-iodo-4-oxo-1,4-dihydro-quinoline-3-carboxylic acid (400 mg, 2.5 equiv.) according to the procedure of Example 4 the title compound was obtained (430 mg).

MS (ES+) m/z: [MH]⁺=1092.6

¹³C-NMR (125 MHz. CDCl₃) δ: 179.09 (C-1), 178.54 (Q-p), 167.35 (Q-o), 149.01 (Q-h) 146.44 (CH═CH), 140.45 (Q-u), 137.89 (Q-s), 134.74 (Q-t), 126.51 (Q-r), 125.86 (Q-f), 116.46 (Q-g), 108.47 (Q-k), 103.77 (C-1′), 102.20 (CH═CH), 94.72 (C-1″), 87.79 (C-4″), 83.25 (C-5), 77.68 (C-3+C-13), 74.22 (C-12), 73.89 (C-6), 73.75 (C-3″), 73.45 (C-11), 71.01 (C-2′), 70.68 (CH₂), 70.02 (C-9), 69.38 (CH₂), 68.92 (CH₂), 67.77 (C-5′), 65.43 (C-3′), 64.74 (C-5″), 62.67 (C-10), 59.25 (3″OMe), 54.14 (CH₂), 45.39 (C-2), 42.37 (C-4), 42.22 (C-7), 40.44 (3′NMe₂), 36.23 (9aNMe), 35.29 (C-2″), 30.61 (CH₂), 29.62 (CH₂), 27.52 (6Me), 26.76 (C-8), 22.01 (8Me), 21.73 (3″Me), 21.67 (5′Me), 21.34 (C-14), 18.26 (5″Me), 16.30 (12Me), 14.58 (2Me), 11.27 (C-15), 9.12 (4Me), 7.31 (10Me).

### Example 7

### 4″-O-{3-[3-(3-Carboxy-1-t-butyl-4-oxo-1,4-dihydro-quinolin-6-yl)-prop-2-en-1-yloxy]-propyl}-azithromycin

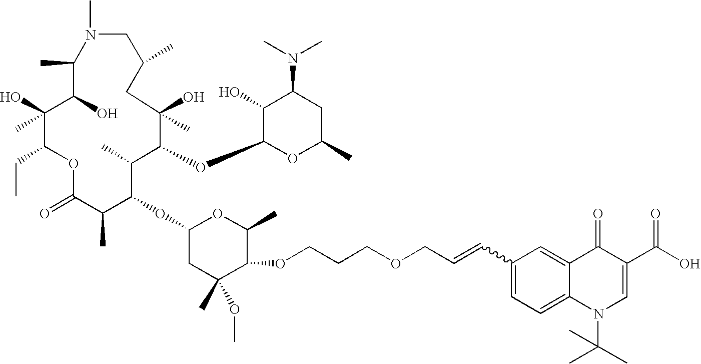

Starting from Intermediate 6 (400 mg, 1 equiv.) and 1-t-butyl-6-iodo-4-oxo-1,4-dihydro-quinoline-3-carboxylic acid (400 mg, 2.5 equiv.) according to the procedure of Example 4 the title compound was obtained (370 mg).

MS (ES+) m/z: [MH]⁺=1090.4.

### Example 8

### 4″-O-{3-[3-(3-Carboxy-1-i-propyl-4-oxo-1,4-dihydro-quinolin-6-yl)-prop-2-en-1-yloxy]-propyl}-azithromycin

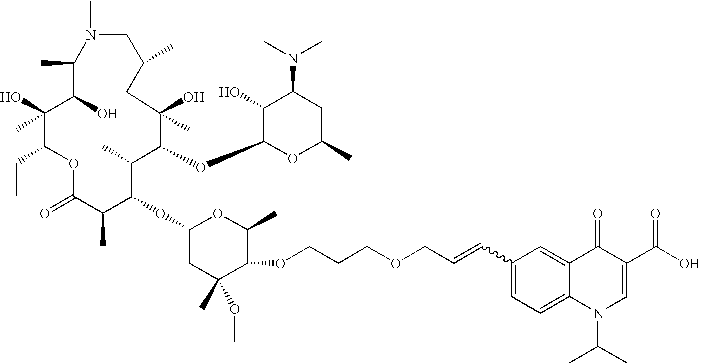

Starting from Intermediate 6 (400 mg, 1 equiv.) and 1-i-propyl-6-iodo-4-oxo-1,4-dihydro-quinoline-3-carboxylic acid (400 mg, 2.5 equiv.) according to the procedure of Example 4 the title compound was obtained (360 mg).

MS (ES+) m/z: [MH]⁺=1076.4.

### Example 9

### 4″-O-{3-[3-(3-Carboxy-1-dimethylaminoeth-2-yl-4-oxo-1,4-dihydro-quinolin-6-yl)-prop-2-en-1-yloxy]-propyl}-azithromycin

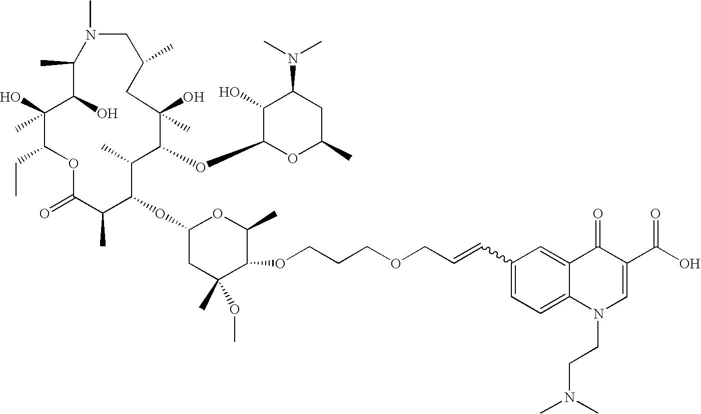

Starting from Intermediate 6 (400 mg, 1 equiv.) and 1-dimethylaminoeth-2-yl-6-iodo-4-oxo-1,4-dihydro-quinoline-3-carboxylic acid (400 mg, 2.5 equiv.) according to the procedure of Example 4 the title compound was obtained (350 mg).

MS (ES+) m/z: [MH]⁺=1105.4.

### Example 10

### 4″-O-{3-[3-(3-Carboxy-1-cyclopropyl-4-oxo-1,4-dihydro-quinolin-6-yl)-propoxy]-propyl}-azithromycin

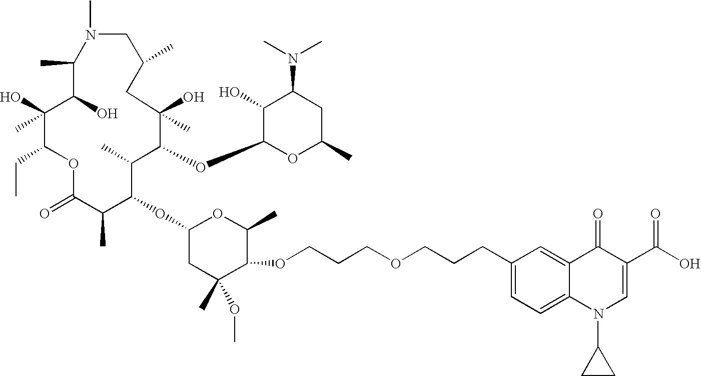

The compound of Example 4 (310 mg, 0.29 mmol) was dissolved in MeOH (25 ml), catalyst 10% Pd/C (0.16 g) was added and hydrogenation performed overnight at 5 bar H₂ pressure. The catalyst was filtered off, solvent evaporated under reduced pressure. The crude product was first purified by column chromatography (using solvent system: DCM/MeOH/NH₃=90:9:0.5) and then precipitated from EtOAc/n-hexane yielding crude title compound (100 mg). A further purification was performed by column chromatography (using solvent system: DCM/MeOH/NH₃=90:9:1.0) and then precipitated from EtOAc/n-hexane yielding the title compound (88 mg).

MS (ES+) m/z: [MH]⁺=1076.5

¹³C-NMR (125 MHz, CDCl₃) δ: 178.85 (C-1), 178.61 (Q-p), 167.23 (Q-o), 147.75 (Q-h), 140.95 (Q-u), 139.47 (Q-s), 134.85 (Q-t), 126.53 (Q-r), 126.21 (Q-f), 116.44 (Q-g), 108.48 (Q-k), 101.84 (C-1′), 94.78 (C-1″), 87.67 (C-4″), 83.34 (C-5), 77.74 (C-3), 77.49 (C-13), 74.20 (C-6), 73.94 (C-12), 73.76 (C-3″), 73.14 (C-11), 71.66 (CH₂), 70.81 (C-2′), 69.82 (C-9), 69.63 (CH₂), 67.72 (CH₂), 67.24 (C-5′), 65.38 (C-3′), 64.85 (C-5″), 62.97 (C-10), 49.61 (3″OMe), 45.37 (C-2), 42.38 (C-4), 42.11 (C-7), 40.09 (3′NMe₂), 36.26 (9aNMe), 35.44 (Q-i), 35.26 (C-2″), 31.94 (CH₂), 31.07 (CH₂), 30.63 (CH₂), 27.29 (6Me), 26.65 (C-8), 22.01 (8Me), 21.68 (3″Me+5′Me), 21.36 (C-14), 18.35 (5″Me), 16.35 (12Me), 14.61 (2Me), 11.24 (C-15), 9.24 (4Me), 8.25 (Q-j), 7.35 (10 Me).

### Example 11

### 4″-O-{3-[3-(3-Carboxy-1-ethyl-4-oxo-1,4-dihydro-quinolin-6-yl)-propoxy]-propyl}-azithromycin

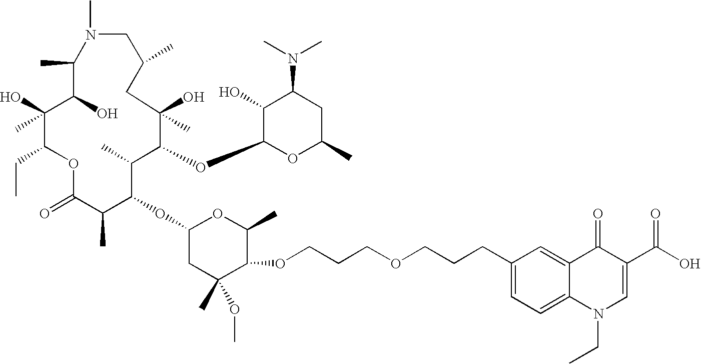

Starting from Example 5 (330 mg, 0.31 mmol) according to the procedure of Example 10, with a further column chromatography purification step using DCM/MeOH/NH3=90:9.0:1.5, the title compound was obtained (190 mg).

MS (ES+) m/z: [MH]⁺=1064.6.

¹³C-NMR (125 MHz, CDCl₃) δ: 179.01 (C-1), 178.42 (Q-p), 167.33 (Q-o), 147.49 (Q-h), 140.82 (Q-u), 137.53 (Q-s), 134.93 (Q-t), 126.69 (Q-r), 126.29 (Q-f), 116.4 (Q-g), 108.82 (Q-k), 102.19 (C-1′), 94.83 (C-1″), 87.75 (C-4″), 83.33 (C-5), 77.75 (C-3), 77.48 (C-13), 74.27 (C-12), 73.93 (C-6), 73.73 (C-3″), 73.58 (C-11), 71.54 (CH₂), 71.08 (C-2′), 70.06 (C-9), 69.65 (CH₂), 67.74 (CH₂), 65.43 (C-3′), 64.85 (C-5″), 62.63 (C-10), 49.74 (CH₂), 49.58 (3″OMe), 45.35 (C-2), 42.31 (C-7), 42.25 (C-4), 40.44 (3′NMe₂), 36.28 (9aNMe), 35.39 (C-2″), 31.94 (CH₂), 31.07 (CH₂), 30.69 (CH₂), 27.51 (6Me), 26.79 (C-8), 22.01 (8Me), 21.72 (3″Me+5′Me), 21.34 (C-14), 18.43 (5″Me), 16.29 (12 Me), 14.75 (2Me+CH₃), 11.26 (C-15), 9.21 (4Me), 7.39 (10Me).

### Example 12

### 4″-O-{3-[3-(3-Carboxy-1-methoxyeth-2-yl-4-oxo-1,4-dihydro-quinolin-6-yl)-propoxy]-propyl}-azithromycin

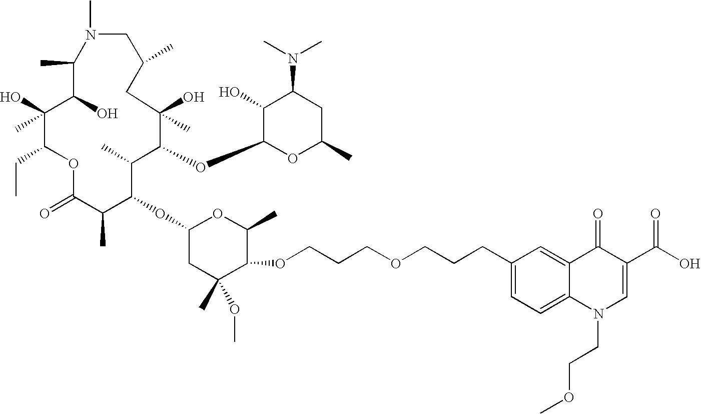

Starting from Example 6 (430 mg, 0.39 mmol) according to the procedure of Example 10 the title compound was obtained (210 mg).

MS (ES+) m/z: [MH]⁺=1094.6.

¹³C-NMR (125 MHz. CDCl₃) δ: 179.03 (C-1), 178.49 (Q-p), 167.27 (Q-o), 149.04 (Q-h), 140.67 (Q-u), 137.89 (Q-s), 134.78 (Q-t), 126.53 (Q-r), 126.21 (Q-f), 116.44 (Q-g), 108.51 (Q-k), 102.09 (C-1′), 94.76 (C-1″), 87.69 (C-4″), 83.32 (C-5), 77.68 (C-3+C-13), 74.19 (C-12), 73.91 (C-6), 73.70 (C-3″), 73.42 (C-11), 71.56 (CH₂), 71.01 (C-2′), 69.99 (C-9), 69.61 (CH₂), 69.36 (CH₂), 69.53 (C-5′), 67.69 (CH₂), 65.41 (C-3′), 64.84 (C-5″), 62.69 (C-10), 59.25 (OCH₃), 54.09 (CH₂), 45.35 (C-2), 42.31 (C-4), 42.22 (C-7), 40.43 (3′NMe₂), 36.22 (9aNMe), 35.31 (C-2″), 31.88 (CH₂), 31.03 (CH₂), 30.64 (CH₂), 27.49 (6Me), 26.74 (C-8), 21.99 (8Me), 21.68 (3″Me+5′Me), 21.32 (C-14), 18.38 (5″Me), 16.29 (12Me), 14.59 (2Me), 11.24 (C-15), 9.18 (4Me), 7.30 (10Me).

### Example 13

### 4″-O-{3-[3-(3-Carboxy-1-t-butyl-4-oxo-1,4-dihydro-quinolin-6-yl)-propoxy]-propyl}-azithromycin

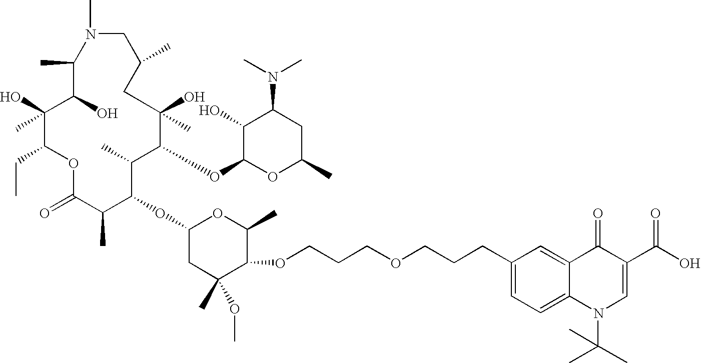

Starting from Example 7 (320 mg, 0.29 mmol) according to the procedure of Example 10 the title compound was obtained (180 mg).

MS (ES+) m/z: [MH]⁺=1092.4.

¹³C-NMR (125 MHz. CDCl₃) δ: 178.84 (C-1), 177.86 (Q-p), 167.77 (Q-o), 145.09 (Q-h), 140.21 (Q-u), 137.73 (Q-s), 133.25 (Q-t), 127.93 (Q-r), 126.58 (Q-f), 120.57 (Q-g), 107.68 (Q-k), 101.95 (C-1′), 94.89 (C-1″), 87.69 (C-4″), 83.52 (C-5), 77.73 (C-3+C-13), 74.28 (C-6), 73.94 (C-12), 73.67 (C-3″), 73.49 (C-11), 71.58 (CH₂), 70.04 (C-2′), 69.96 (C-9), 69.71 (CH₂), 67.74 (CH₂), 67.59 (C-5′), 64.46 (C-3′), 64.85 (C-5″), 64.62 (C(C(CH₃)₃), 62.09 (C-10), 49.55 (3″OMe), 45.28 (C-2), 42.06 (C-7), 42.05 (C-4), 40.65 (3′NMe₂), 36.35 (9aNMe), 35.38 (C-2″), 31.69 (CH₂), 31.00 (CH₂), 30.92 (C(CH₃)₃), 30.69 (CH₂), 4′ (n.d.), 27.41 (C-8), 26.74 (6Me), 22.02 (8Me), 21.69 (3″Me+5′Me), 21.33 (C-14), 18.44 (5″Me), 16.28 (12Me), 14.73 (2Me), 11.27 (C-15), 9.36 (4Me), 7.52 (10Me).

### Example 14

### 4″-O-{3-[3-(3-Carboxy-1-i-propyl-4-oxo-1,4-dihydro-quinolin-6-yl)-propoxy]-propyl}-azithromycin

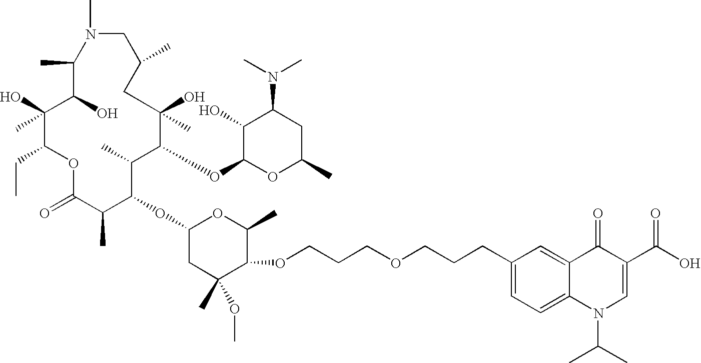

Starting from Example 8 (310 mg, 0.29 mmol) according to the procedure of Example 10 the title compound was obtained (150 mg).

MS (ES+) m/z: [MH]⁺=1078.4.

### Example 15

### 4″-O-{3-[3-(3-Carboxy-1-dimethylaminoethyl-4-oxo-1,4-dihydro-quinolin-6-yl)-propoxy]-propyl}-azithromycin

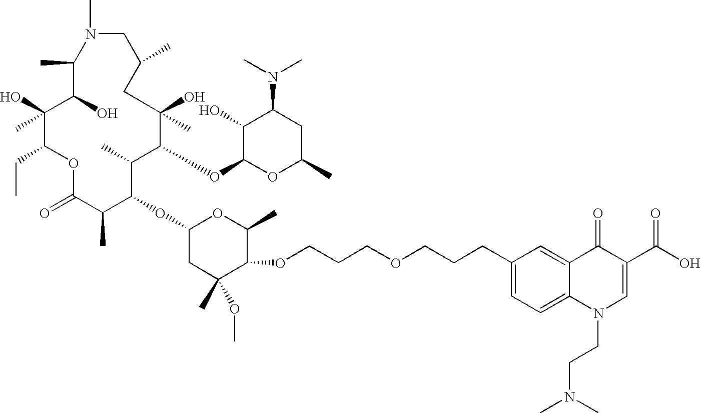

Starting from Example 9 (300 mg, 0.27 mmol) according to the procedure of Example 10 the title compound was obtained (160 mg).

MS (ES+) m/z: [MH]⁺=1107.6.

### Example 16

### 2′-O-Acetyl-4″-O-{2-[3-(3-carboxy-1,4-dihydro-1-dimethylamino-4-oxo-6-quinolinyl)prop-2-enyl]-oxyethyl}-azithromycin 11,12-carbonate ethyl ester

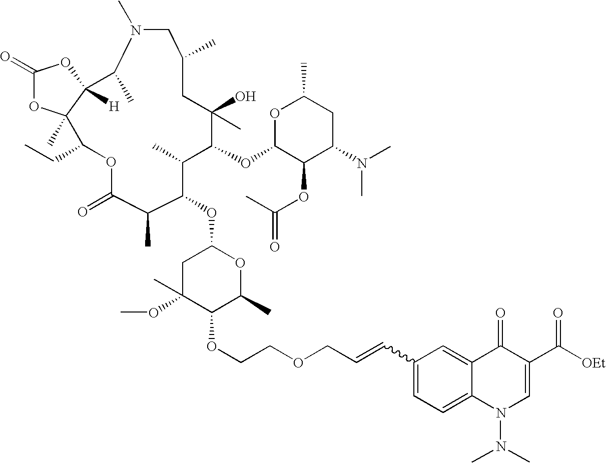

A solution of triethylamine (0.63 ml) in dry acetonitrile (4 ml) was degassed by bubbling argon while ultrasonicating for 5 min. To this solution was added ethyl 1-(dimethylamino)-6-iodo-4-oxo-1,4-dihydro-3-quinolinecarboxylate (0.35 g), triphenyl phosphine (0.008 g) and sodium bicarbonate (0.077 g). A degassed solution of 2′-O-acetyl-4″-O-(2-allyloxyethyl)-azithromycin 11,12-carbonate (0.41 g) in dry acetonitrile (10 mL) was added to the above solution followed by palladium diacetate (0.024 g). The reaction was refluxed for 2 h after which further palladium acetate (0.024 g) was added. After a further 2 h, further palladium acetate (0.024 g) was added. The reaction mixture was refluxed for a further 1 h, cooled, filtered through celite and evaporated under reduced pressure to dryness to give the crude title compound (0.8 g); ESMS m/z 1159.7 [M+H]⁺.

### Example 17

### 2′-O-Acetyl-4″-O-{2-[3-(3-carboxy-1,4-dihydro-1-dimethylamino-4-oxo-6-quinolinyl)propyl]-oxyethyl}-azithromycin 11,12-carbonate ethyl ester

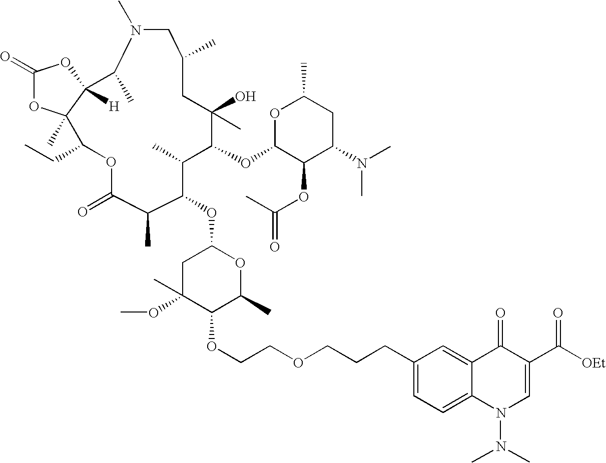

Crude 2′-O-acetyl-4″-O-{2-[3-(3-carboxy-1,4-dihydro-1-dimethylamino-4-oxo-6-quinolinyl)prop-2-enyl]-oxyethyl}-azithromycin 11,12-carbonate ethyl ester (0.8 g) in a 1:1 mixture of DCM and methanol (80 mL) was treated with 10% Pd/C (0.5 g). After 5 min the catalyst was filtered through Celite and replaced with fresh 10% Pd/C (0.5 g) then hydrogenated at 20° C. and 1 atm for 5 h. The reaction was filtered through Celite and concentrated to give the crude title compound (0.62 g); ESMS m/z 1161.7 [M+H]⁺.

### Example 18

### 4″-O{2-[3-(3-Carboxy-1,4-dihydro-1-dimethylamino-4-oxo-6-quinolinyl)propyl]-oxyethyl}-azithromycin

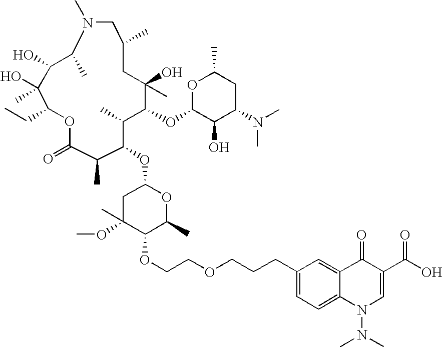

Crude 2′-O-acetyl-4″-O-{2-[3-(3-carboxy-1,4-dihydro-1-dimethylamino-4-oxo-6-quinolinyl)propyl]-oxyethyl}-azithromycin 11,12-carbonate ethyl ester (0.62 g) in acetonitrile (20 mL) was treated with 10% aqueous solution of potassium carbonate (10 mL) and heated at 85° C. for 40 h. The mixture was cooled down then the pH adjusted to 6 by adding a 10% aqueous solution of citric acid. Acetonitrile was evaporated under reduced pressure and the residue was partitioned between DCM and water. The organic phase was dried, concentrated and the residue chromatographed eluting with 0-10% (10:1 methanol/0.880 ammonia) in DCM to give the impure product. Further purification by preparative reverse phase HPLC (MeCN/H₂O/0.1% HCO₂H eluent) followed by normal phase chromatography as above gave the pure title compound as a white solid (0.095 g); ESMS m/z 1065.5 [M+H]⁺.

### Example 19

### 2′-O-Acetyl-4″-O-{2-[3-(3-carboxy-1,4-dihydro-1-ethyl-4-oxo-6-quinolinyl)prop-2-enyl]-oxyethyl}-azithromycin 11,12-carbonate ethyl ester

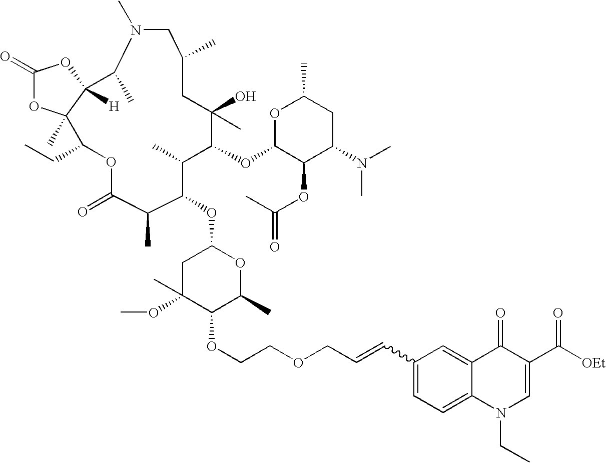

A solution of triethylamine (0.68 mL) in dry acetonitrile (5 mL) was degassed by bubbling argon while ultrasonicating for 5 min. To this solution was added ethyl 1-ethyl-6-iodo-4-oxo-1,4-dihydro-3-quinolinecarboxylate (0.364 g), triphenyl phosphine (0.0085 g) and sodium bicarbonate (0.083 g). A degassed solution of 2′-O-acetyl-4″-O-(2-allyloxyethyl)-azithromycin 11,12-carbonate (0.442 g) in dry acetonitrile (10 mL) was added to the above solution followed by palladium diacetate (0.026 g). The reaction was refluxed for 2 h after which further palladium acetate (0.026 g) was added. After a further 2 h, further palladium acetate (0.026 g) was added. The reaction was refluxed for a further 1 h, cooled, filtered through celite and evaporated under reduced pressure to dryness to give the crude product which was purified by chromatography over silica gel eluting with 0-10% (10:1 methanol/0.880 ammonia) in DCM to yield the title compound (0.5 g);

ESMS m/z 573.3 [M+2H]⁺⁺.

### Example 20

### 2′-O-Acetyl-4″-O-{2-[3-(3-carboxy-1,4-dihydro-1-ethyl-4-oxo-6-quinolinyl)propyl]-oxyethyl}-azithromycin 11,12-carbonate ethyl ester

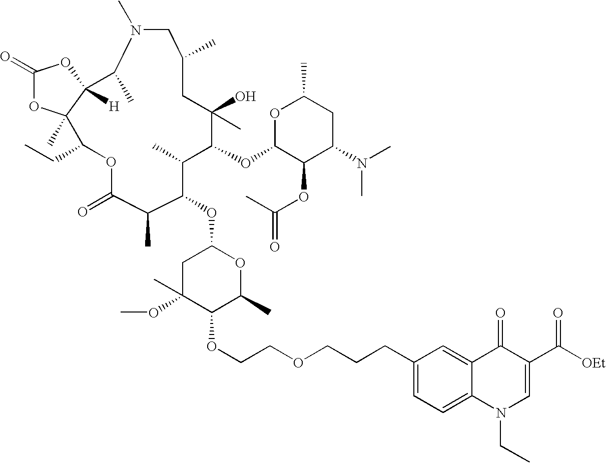

2′-O-Acetyl-4″-O-{2-[3-(3-carboxy-1,4-dihydro-1-ethyl-4-oxo-6-quinolinyl)prop-2-enyl]-oxyethyl}-azithromycin 11,12-carbonate ethyl ester (0.5 g) in methanol (40 mL) was treated with 10% Pd/C (0.17 g). After 5 min the catalyst was filtered through celite and replaced with fresh 10% Pd/C (0.17 g) then hydrogenated at 20° C. and 1 atm overnight. The reaction mixture was filtered through celite and concentrated to give the crude title compound which was used without purification (0.5 g); ESMS m/z 574.3 [M+2H]⁺⁺.

### Example 21

### 4″-O-{2-[3-(3-Carboxy-1,4-dihydro-1-ethyl-4-oxo-6-quinolinyl)propyl]-oxyethyl}-azithromycin

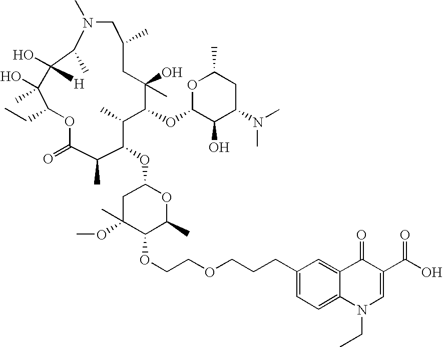

Crude 2′-O-acetyl-4″-O-{2-[3-(3-carboxy-1,4-dihydro-1-ethyl-4-oxo-6-quinolinyl) propyl]-oxyethyl}-azithromycin 11,12-carbonate ethyl ester (0.5 g) in acetonitrile (25 mL) was treated with 10% aqueous solution of potassium carbonate (10 mL) and heated at 80° C. for 45 h. The mixture was cooled down then the pH adjusted to 6 by adding a 10% aqueous solution of citric acid. Acetonitrile was evaporated under reduced pressure and the residue was partitioned between DCM and water. The organic phase was dried, concentrated and the residue chromatographed over silica gel eluting with 0-10% (10:1 methanol/0.880 ammonia) in DCM to give the title compound as a white solid (0.060 g);

ESMS m/z 1048.7 [M−H]⁻.

### Example 22

### 2′-O-Acetyl-4″-O-{5-[3-(3-carboxy-1-ethyl-4-oxo-1,4-dihydro-quinolin-6-yl)-prop-2-en-1-yloxy]-pentyl}-azithromycin-11,12-cyclic carbonate

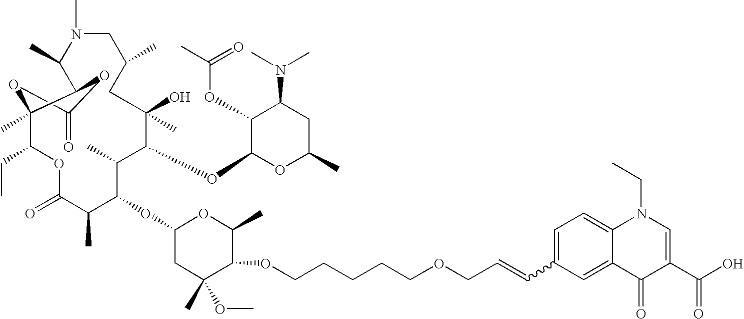

To the degassed solution of Intermediate 22 (0.156 g, 0.17 mmol) in DMF (4 ml), Pd(OAc)2 (0.0075 g, 0.2 equiv.) and TOTF (0.020 g, 0.4 equiv.) were added and stirred at room temperature for 15 min. Then, 1-ethyl-6-iodo-4-oxo-1,4-dihydro-quinoline-3-carboxylic acid (0.142 g, 0.41 mmol) and Et3N (92.4 ml, 4 equiv.) were added, the mixture stirred at 65° C. for 1.5 hour and then at 75° C. overnight. After filtration EtOAc (30 ml) was added and washed with water (3×20 ml). Evaporation of organic layer yielded the title product (254 mg).

MS; m/z (ES): 1159.4 [MH]+.

### Example 23

### 4″-O-{5-[3-(3-carboxy-1-ethyl-4-oxo-1,4-dihydro-quinolin-6-yl)-prop-2-en-1-yloxy]-pentyl}-azithromycin

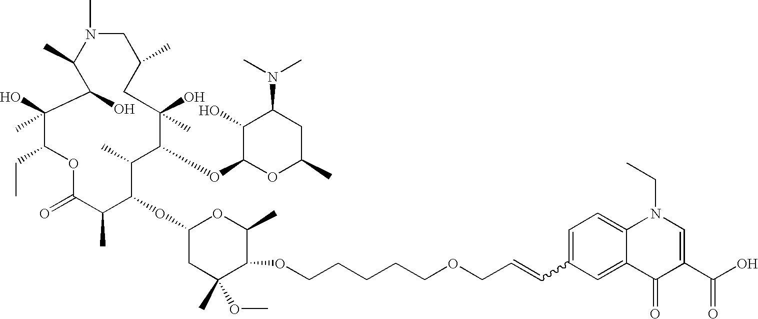

To a solution of Example 22 (254 mg, 0.22 mmol) in MeOH (15 ml) and water (5 ml) K₂CO₃ (0.546 g, 3.96 mmol) was added and reaction mixture stirred at 50° C. overnight. The solvent was evaporated, DCM (30 ml) added, washed with water (2×20 ml), organic layer dried over K₂CO₃ and evaporated under reduced pressure yielding the title product (211 mg).

MS; m/z (ES): 1090.8 [MH]⁺.

### Example 24

### 4″-O-{5-[3-(3-carboxy-1-ethyl-4-oxo-1,4-dihydro-quinolin-6-yl)-propyloxy]-pentyl}-azithromycin

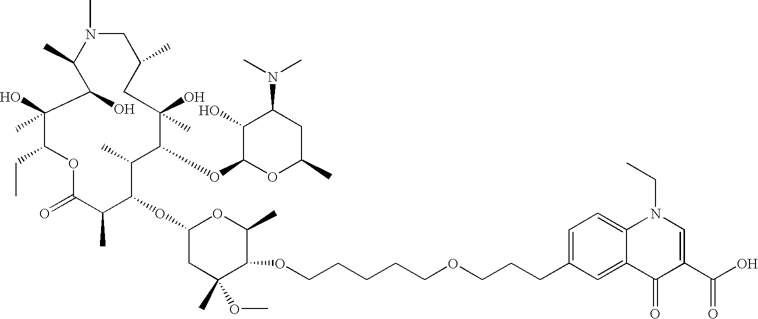

Hydrogenation of Example 23 (0.211 g, 0.19 mmol) in MeOH (30 ml) with 10% Pd/C (0.1 g) in Parr apparatus at 5 bar overnight gave the crude product (0.131 g) which was first purified by column chromatography (DCM/MeOH/NH₃=90:9:1.5) and then precipitated from EtOAc/n-hexane yielding the title product (23.5 mg).

MS; m/z (ES): 1093.0 [MH]⁺.

¹³C-NMR (75 MHz, CDCl₃) δ: 178.3, 177.85, 166.73, 146.88, 140.29, 137.5, 134.34, 126.14, 125.79, 115.75, 108.27, 101.76, 94.62, 87.19, 82.79, 77.69, 76.94, 73.12, 70.56, 70.36, 69.53, 69.03, 67.31, 64.72, 64.27, 49.14, 49.06, 44.60, 41.86, 39.85, 35.89, 35.01, 31.36, 30.54, 29.63, 29.12, 26.82, 26.23, 22.18, 21.42, 21.15, 20.68, 18.01, 15.67, 14.49, 14.18, 10.70, 8.80, 7.00.

### Example 25

### 4″-O-{4-[3-(3-carboxy-1-ethyl 4-oxo-1,4-dihydro-quinolin-6-yl)-prop-2-en-1-yloxy]-butyl}-azithromycin

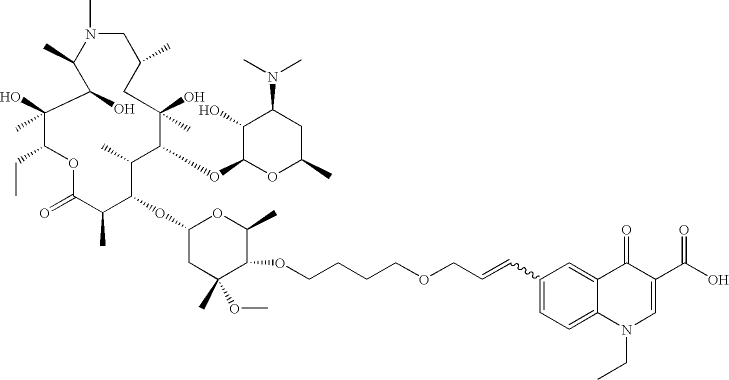

Starting from Intermediate 25b and 1-ethyl-6-iodo-4-oxo-1,4-dihydro-quinoline-3-carboxylic acid according to the procedure described for Example 1 the title compound is obtained.

### Example 26

### 4″-O-{4-[3-(3-carboxy-1-cyclopropyl-4-oxo-1,4-dihydro-quinolin-6-yl)-prop-2-en-1-yloxy]-butyl}-azithromycin

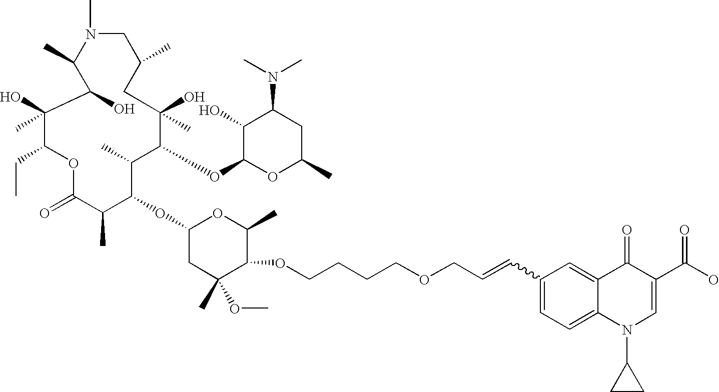

Starting from Intermediate 25b and 1-cyclopropyl-6-iodo-4-oxo-1,4-dihydro-quinoline-3-carboxylic acid according to the procedure described for Example 1 the title compound is obtained.

### Example 27

### 4″-O-{4-[3-(3-carboxy-1-dimethylamino-4-oxo-1,4-dihydro-quinolin-6-yl)-prop-2-en-1-yloxy]-butyl}-azithromycin

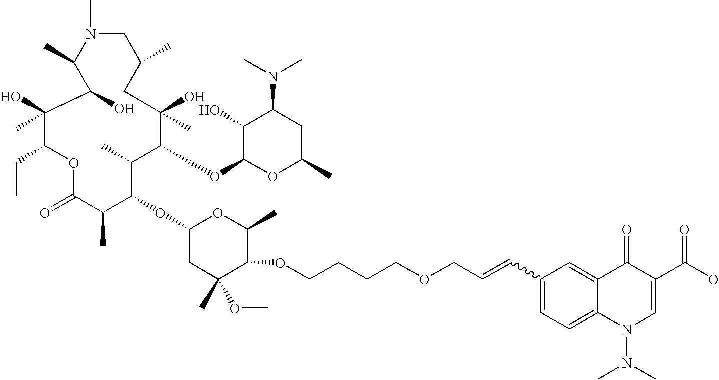

Starting from Intermediate 25b and 1-dimethylamino-6-iodo-4-oxo-1,4-dihydro-quinoline-3-carboxylic acid according to the procedure described for Example 1 the title compound is obtained.

### Example 28

### 4″-O-{4-[3-(3-carboxy-1-methoxyeth-2-yl-4-oxo-1,4-dihydro-quinolin-6-yl)-prop-2-en-1-yloxy]-butyl}-azithromycin

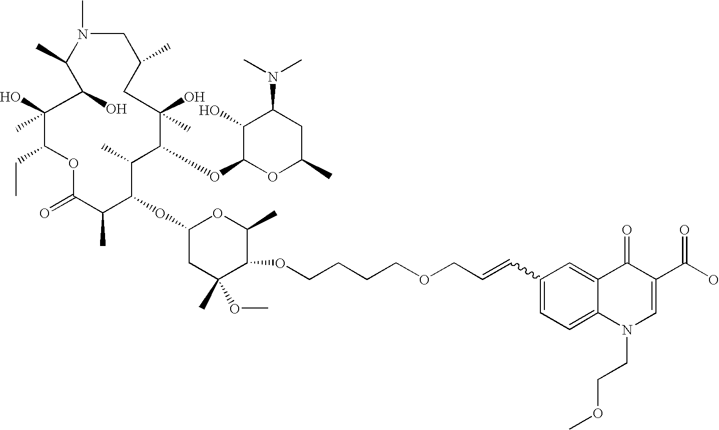

Starting from Intermediate 25b and 1-methoxyeth-2-yl-6-iodo-4-oxo-1,4-dihydro-quinoline-3-carboxylic acid according to the procedure described for Example 1 the title compound is obtained.

### Example 29

### 4″-O-{4-[3-(3-carboxy-1-ethyl-4-oxo-1,4-dihydro-quinolin-6-yl)-propyloxy]-butyl}-azithromycin

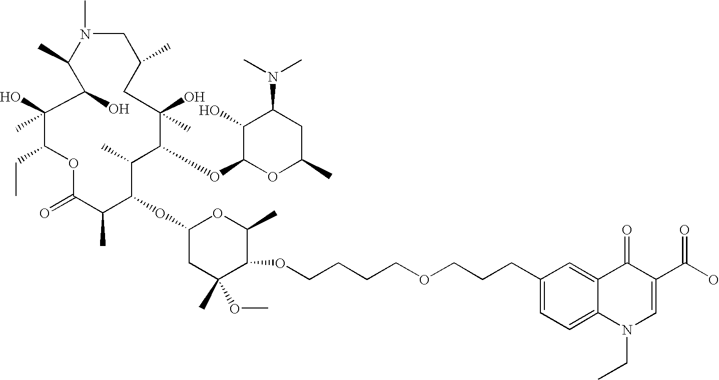

Starting from Example 25 according to the procedure described for Example 3, Method A the title compound is obtained.

### Example 30

### 4″-O-{4-[3-(3-carboxy-1-cyclopropyl-4-oxo-1,4-dihydro-quinolin-6-yl)-propyloxy]-butyl}-azithromycin

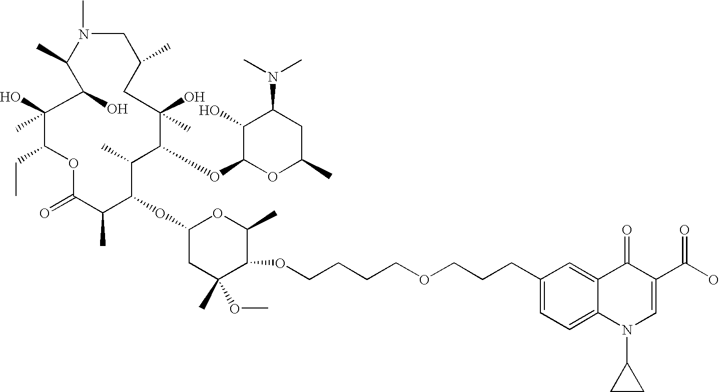

Starting from Example 26 according to the procedure described for Example 3, Method A the title compound is obtained.

### Example 31

### 4″-O-{4-[3-(3-carboxy-1-dimethylamino-4-oxo-1,4-dihydro-quinolin-6-yl)-propyloxy]-butyl}-azithromycin

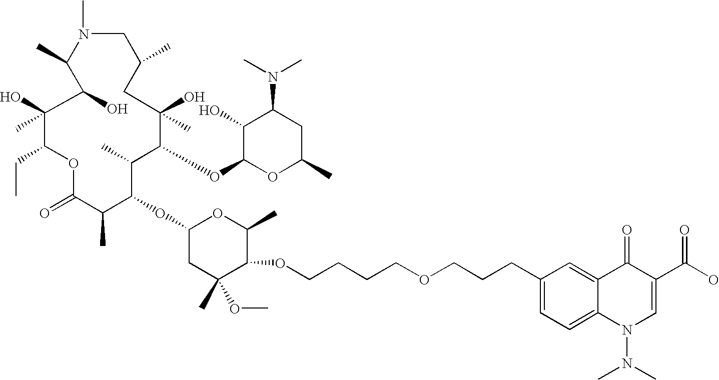

Starting from Example 27 according to the procedure described for Example 3, Method A the title compound is obtained.

### Example 32

### 4″-O-{4-[3-(3-carboxy-1-methoxyeth-2-yl-4-oxo-1,4-dihydro-quinolin-6-yl)-propyloxy]-butyl}-azithromycin

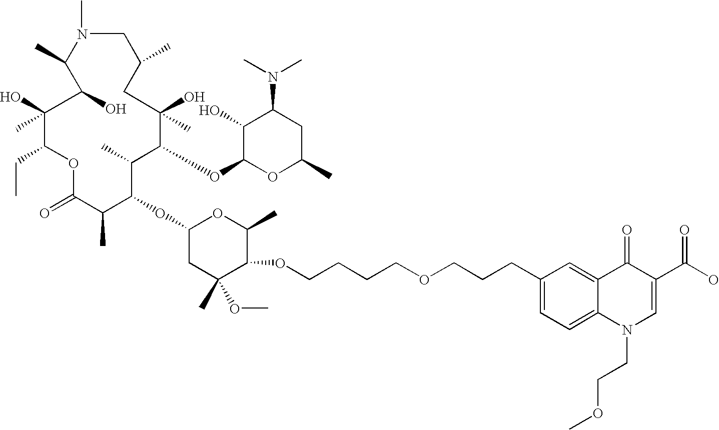

Starting from Example 28 according to the procedure described for Example 3, Method A the title compound is obtained.

### Examples 33, 34

### 2′-O-Acetyl-4″-O-{2-[3-(6-carboxy-3,3-dimethyl-7-oxo-1H,7H-[1,3oxazino]5,4,3-ij]-9-quinolinyl)prop-2-enyl]-oxyethyl}-azithromycin 11,12-carbonate ethyl ester

### 2′-O-Acetyl-4″-O-{2-[3-(6-carboxy-3,3-dimethyl-7-oxo-1H,7H-[1,3oxazino]5,4,3-ij]-9-quinolinyl)prop-1-enyl]-oxyethyl}-azithromycin 11,12-carbonate ethyl ester

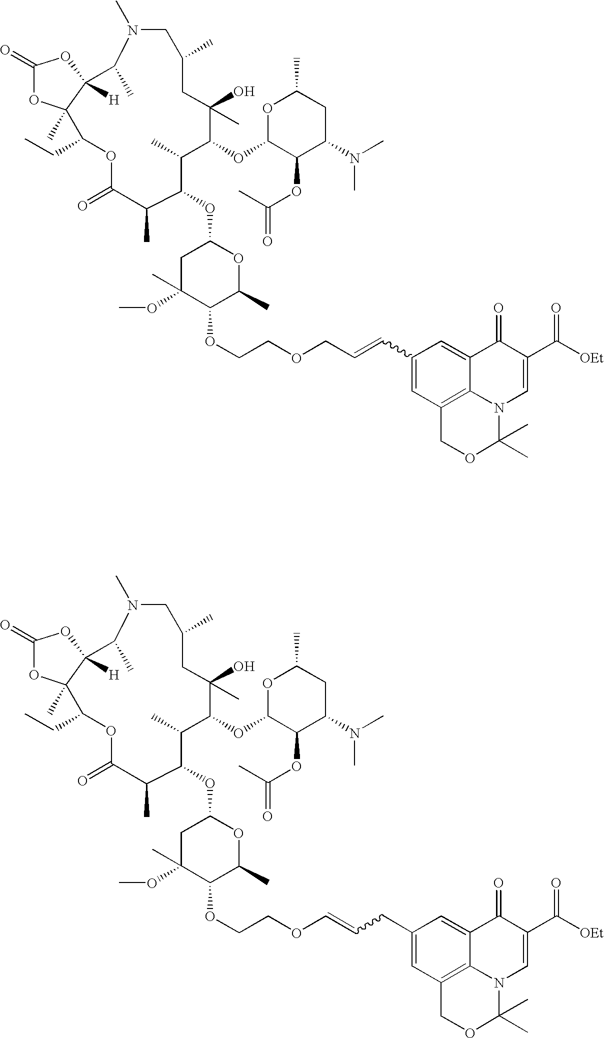

A solution of 2′-O-acetyl-4″-O-(2-allyloxyethyl)-azithromycin 11,12-carbonate (0.29 g, 0.32 mmol) in acetonitrile (6.4 mL) was degassed. Triethylamine (0.64 mL) was added followed by palladium (II) diacetate (0.03 g) and 9-iodo-3,3-dimethyl-7-oxo-1H,7H-[1,3]oxazino[5,4,3-ij]quinolone-6-carboxylic acid ethyl ester (0.37 g, 0.96 mmol). The resultant mixture was heated at 80° C. After 2 h further batches of palladium (II) diacetate (0.02 g) and 9-iodo-3,3-dimethyl-7-oxo-1H,7H-[1,3]oxazino[5,4,3-ij]quinolone-6-carboxylic acid ethyl ester (0.066 g) were added and the mixture heated for another 2.5 h. After this time the reaction was cooled and concentrated, the crude was purified by chromatography over silica gel eluting with 0-10% (10:1 methanol/0.880 ammonia) in DCM to the title compounds as a mixture of isomers as a white solid (0.29 g); ESMS m/z 1158.7 [M+H]⁺.

### Example 35

### 2′-O-Acetyl-4″-O-{2-[3-(6-carboxy-3,3-dimethyl-7-oxo-1H,7H-[1,3oxazino]5,4,3-ij]-9-quinolinyl)prop-1-yl]-oxyethyl}-azithromycin 11,12-carbonate ethyl ester

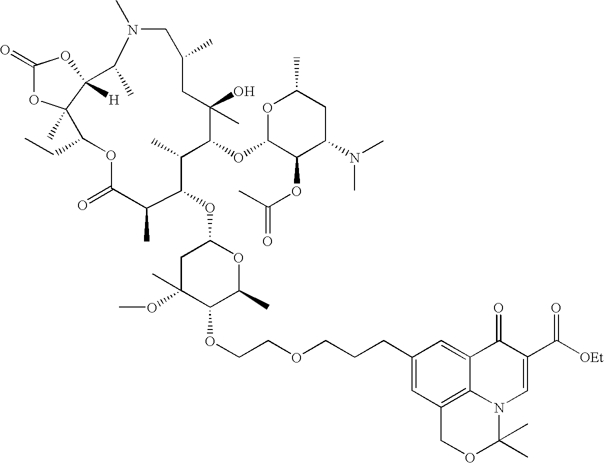

A solution of a mixture of 2′-O-acetyl-4″-O-{2-[3-(6-carboxy-3,3-dimethyl-7-oxo-1H,7H-[1,3oxazino]5,4,3-ij]-9-quinolinyl)prop-2-enyl]-oxyethyl}-azithromycin 11,12-carbonate ethyl ester and 2′-O-acetyl-4″-O-{2-[3-(6-carboxy-3,3-dimethyl-7-oxo-1H,7H-[1,3oxazino]5,4,3-ij]-9-quinolinyl)prop-1-enyl]-oxyethyl}-azithromycin 11,12-carbonate ethyl ester (0.29 g, 0.25 mmol) in DCM (20 mL) was hydrogenated over 10% palladium charcoal (0.15 g) at atmospheric pressure and room temperature. After 5 h the mixture was filtered and the filtrate evaporated to yield the title compound (0.24 g);

ESMS m/z 1158.3 [M−H]⁻.

### Example 36

### 4″-O-{2-[3-(6-carboxy-3,3-dimethyl-7-oxo-1H,7H-[1,3oxazino]5,4,3-ij]-9-quinolinyl)prop-1-yl]-oxyethyl}-azithromycin

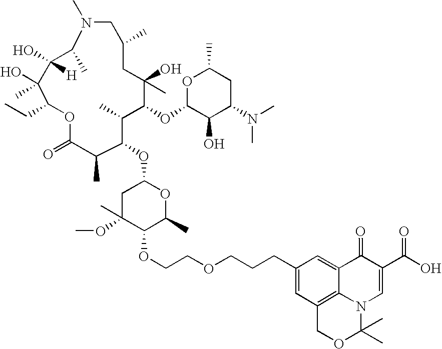

A mixture of acetonitrile (10 mL), 10% aqueous potassium carbonate solution (5 mL) and 2′-O-acetyl-4″-O-{2-[3-(6-carboxy-3,3-dimethyl-7-oxo-1H,7H-[1,3oxazino]5,4,3-ij]-9-quinolinyl)prop-1-yl]-oxyethyl}-azithromycin 11,12-carbonate ethyl ester (0.24 g, 0.207 mmol) was heated at 80° C. After 6.5 h the mixture was cooled, partially concentrated and 10% aqueous citric acid added to achieve pH 6. The solution was then extracted with DCM/chloroform (1:1). The resultant crude product was purified by chromatography over silica gel eluting with 0-15% (10:1 methanol/0.880 ammonia) in DCM to yield two partially purified fractions. These were subjected MDAP and subsequent chromatography over silica gel eluting with 0-15% (10:1 methanol/0.880 ammonia) in DCM. The resultant fractions were freeze-dried to yield the title compound as a solid (0.05 g);

ESMS m/z 1090.6 [M−H]⁻.

### Example 37

### 4″-O-{3-[4-(3-Carboxy-1-dimethylamino-4-oxo-1,4-dihydro-quinolin-6-yl)-but-3-ynyloxy]-propyl}-azithromycin

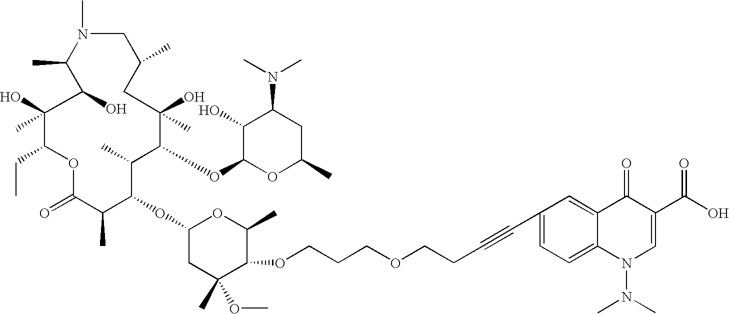

To a suspension of 1-dimethylamino-6-iodo-4-oxo-1,4-dihydro-quinoline-3-carboxylic acid (0.37 g, 1 mmol) in MeCN (5 ml), CuI (19.8 mg, 0.1 mmol) and Et₃N (0.72 ml, 5.2 mmol) were added and the reaction mixture stirred at room temperature for 20 minutes under N₂ flow and then heated to 50° C. A solution of 4″-O-(3-but-3-ynyloxy-propyl)azithromycin (0.45 g, 0.52 mmol) in MeCN (5 ml) and Pd(PPh₃)₂Cl₂ (18.2 mg, 0.026 mmol) were added and stirring was continued for 16 hours under N₂ flow at 50° C. After MeCN was evaporated EtOAc (30 ml) was added and impurities removed by filtration. The filtrate was washed with saturated aqueous NaHCO₃ (2×40 ml) followed by extraction with water (30 ml) at pH 3. Then the aqueous layer was extracted with DCM (20 ml) at pH 4. The pH of the aqueous layer was adjusted to 5.5 and extracted with DCM (2×20 ml). To the combined organic layers (pH 5.5) water was added (20 ml) and pH adjusted to 10. The organic layer at pH 10 was evaporated to give the crude product which was first precipitated from EtOAc/n-hexane and then purified by column chromatography (eluent DCM/MeOH:/NH₃=90:15:1.5) to give the title compound as a white foamy solid (220 mg).

MS (ES+) m/z: [MH]⁺=1089.8.

### Example 38

### 4″-O-{3-[4-(3-Carboxy-1-ethyl-4-oxo-1,4-dihydro-quinolin-6-yl)-but-3-ynyloxy]-propyl}-azithromycin

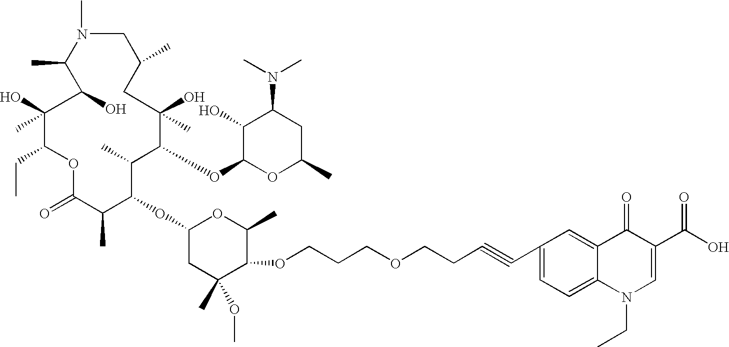

Starting from 4″-O-(3-but-3-ynyloxy-propyl)azithromycin (0.45 g, 0.52 mmol) and 1-ethyl-6-iodo-4-oxo-1,4-dihydro-quinoline-3-carboxylic acid (0.35 g, 1 mmol) according to procedure described for Example 37 the title compound was obtained as a white foamy solid (210 mg).

MS (ES+) m/z: [MH]⁺=1074.7.

### Example 39

### 4″-O-{3-[5-(3-Carboxy-1-dimethylamino-4-oxo-1,4-dihydro-quinolin-6-yl)-pent-4-ynyloxy]-propyl}-azithromycin

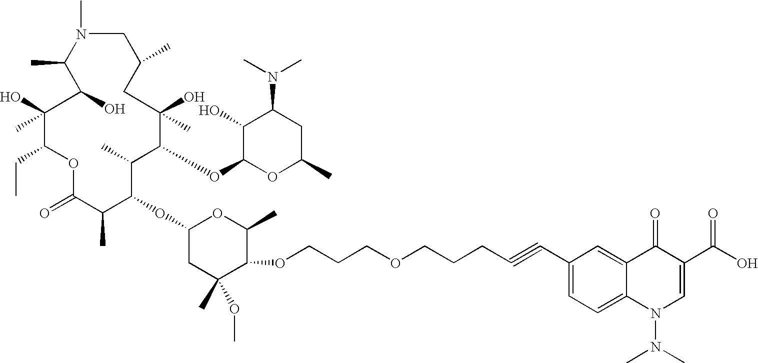

Starting from 4″-O-(3-pent-4-ynyloxy-propyl)azithromycin (0.45 g, 0.51 mmol) and 1-dimethylamino-6-iodo-4-oxo-1,4-dihydro-quinoline-3-carboxylic acid (0.37 g, 1 mmol) according to procedure described for Example 37 the title compound was obtained as a white foamy solid (240 mg).

MS (ES+) m/z: [MH]⁺=1103.5.

### Example 40

### 4″-O-{3-[5-(3-Carboxy-1-ethyl-4-oxo-1,4-dihydro-quinolin-6-yl)-pent-4-ynyloxy]-propyl}-azithromycin

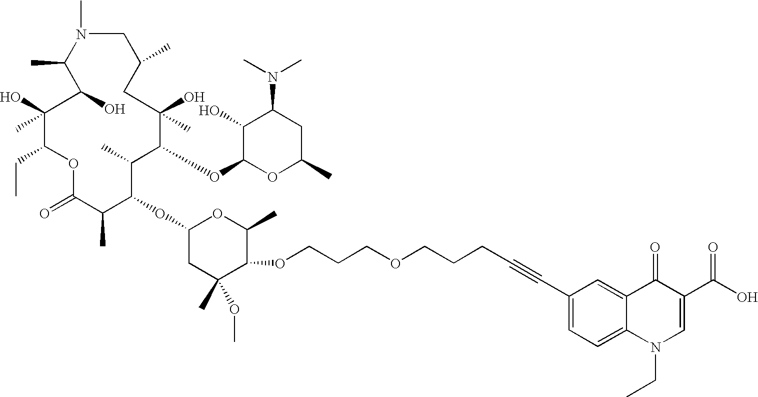

Starting from 4″-O-(3-pent-4-ynyloxy-propyl)azithromycin (0.45 g, 0.51 mmol) and 1-ethyl-6-iodo-4-oxo-1,4-dihydro-quinoline-3-carboxylic acid (0.35 g, 1 mmol) according to procedure described for Example 37 the title compound was obtained as a white foamy solid (170 mg).

MS (ES+) m/z: [MH]⁺=1088.5.

### Example 41

### 4″-O-{3-[4-(3-Carboxy-1-cyclopropyl-4-oxo-1,4-dihydro-quinolin-6-yl)-but-3-ynyloxy]-propyl}-azithromycin

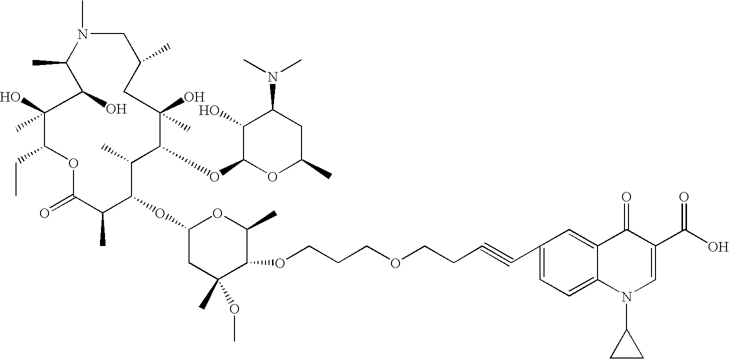

Starting from 4″-O-(3-but-3-ynyloxy-propyl)azithromycin (0.40 g, 0.47 mmol) and 1-cyclopropyl-6-iodo-4-oxo-1,4-dihydro-quinoline-3-carboxylic acid (0.33 g, 0.94 mmol) according to procedure described for Example 37 the title compound was obtained as a white foamy solid (212 mg).

MS (ES+) m/z: [MH]⁺=1086.9.

### Example 42

### 4″-O-{3-[4-(3-Carboxy-1-dimethylamino-4-oxo-1,4-dihydro-quinolin-6-yl)-butoxy]-propyl}-azithromycin

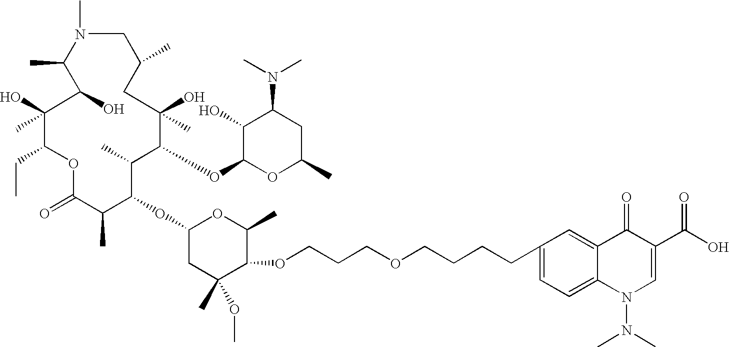

The compound of Example 37 (190 mg, 0.17 mmol) was dissolved in MeOH (20 ml), catalyst 10% Pd/C (40 mg) was added and hydrogenation performed over 15 hours at 2 bar H₂ pressure. The catalyst was filtrated off, solvent evaporated in vacuum. The crude product was first purified by column chromatography (using solvent system: DCM/MeOH/NH₃=90:9:1.5), precipitated from EtOAc/n-hexane and dried at 40° C. for 3 hours yielding the title compound as a white solid (130 mg).

MS (ES+) m/z: [MH]⁺=1093.8.

### Example 43

### 4″-O-{3-[4-(3-Carboxy-1-ethyl-4-oxo-1,4-dihydro-quinolin-6-yl)-butoxy]-propyl}-azithromycin

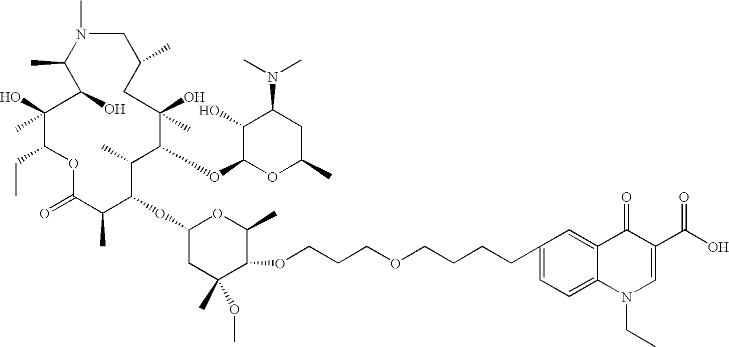

According to the procedure for Example 42 starting from Example 38 (180 mg, 0.17 mmol) and 10% Pd/C (35 mg) as catalyst the title compound was obtained as a white solid (110 mg).

MS (ES+) m/z: [MH]⁺=1078.7.

### Example 44

### 4″-O-{3-[5-(3-Carboxy-1-dimethylamino-4-oxo-1,4-dihydro-quinolin-6-yl)-pentoxy]-propyl}-azithromycin

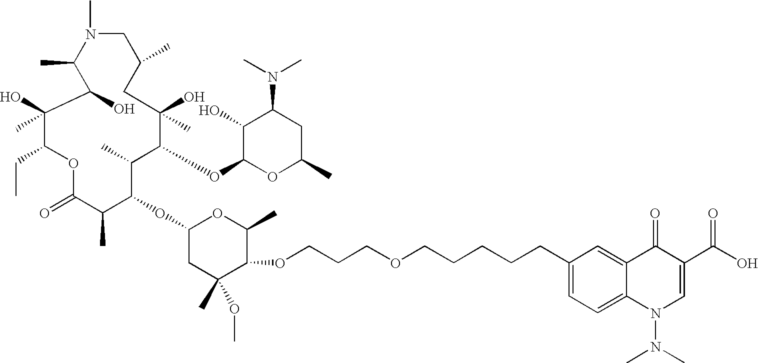

According to the procedure for Example 42 starting from Example 39 (200 mg, 0.18 mmol) and 10% Pd/C (50 mg) as catalyst the title compound was obtained as a white solid (93 mg).

MS (ES+) m/z: [MH]⁺=1107.7.

### Example 45

### 4″-O-{3-[5-(3-Carboxy-1-ethyl-4-oxo-1,4-dihydro-quinolin-6-yl)-pentoxy]-propyl}-azithromycin

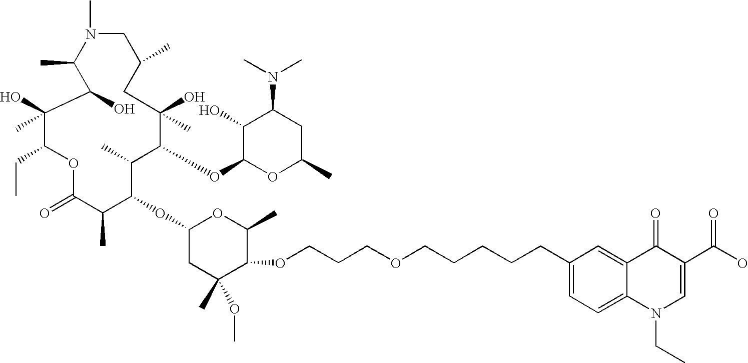

According to the procedure for Example 42 starting from Example 40 (130 mg, 0.12 mmol) and 10% Pd/C (35 mg) as catalyst the title compound was obtained as a white solid (88 mg).

MS (ES+) m/z: [MH]⁺=1092.9.

### Example 46

### 4″-O-{3-[4-(3-Carboxy-1-cyclopropyl-4-oxo-1,4-dihydro-quinolin-6-yl)-butoxy]-propyl}-azithromycin

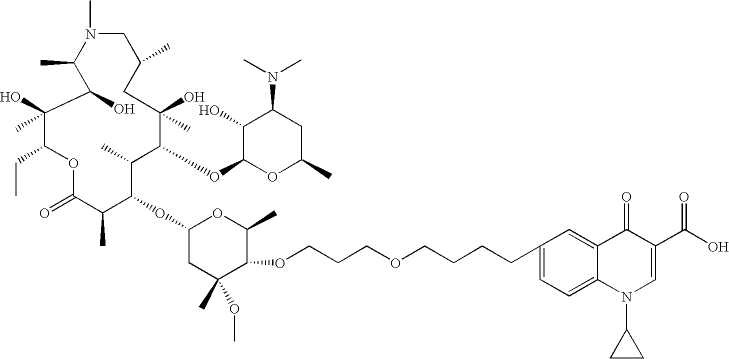

According to the procedure for Example 42 starting from Example 41 (180 mg, 0.17 mmol) and 10% Pd/C (35 mg) as catalyst the title compound was obtained as a white solid (114 mg).

MS (ES+) m/z: [MH]⁺=1090.9.

### Example 47

### 4″-O-{3-[3-(5-Carboxy-3,3-dimethyl-6-oxo-1H,6H-2-oxa-3a-aza-phenalen-8-yl)-prop-2-ynyloxy]-propyl}-azithromycin

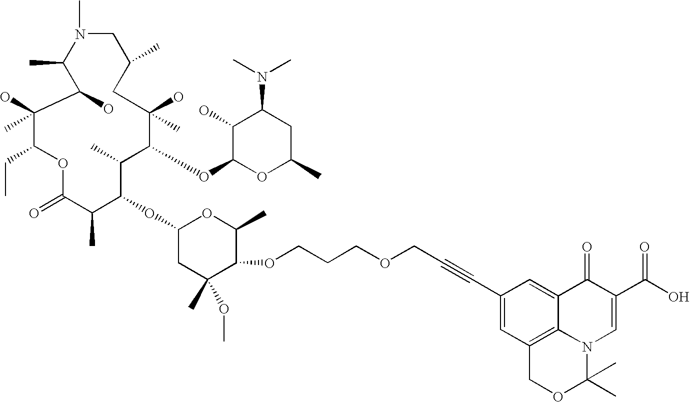

To a suspension of 8-iodo-3,3-dimethyl-6-oxo-1H,6H-2-oxa-3a-aza-phenalene-5-carboxylic acid (also known as 9-iodo-3,3-dimethyl-7-oxo-1H,7H-[1,3]oxazino[5,4,3-ij]quinoline-6-carboxylic acid, Intermediate 62) (364 mg, 0.47 mmol) in EtOH (5 ml), CuI (17.7 mg, 0.09 mmol) and Et₃N (0.66 ml, 4.7 mmol) were added and the reaction mixture was stirred at room temperature for 20 minutes under N₂ flow and then heated to 50° C. A solution of Intermediate 8 (4″-O-(3-prop-2-ynyloxy-propyl)azithromycin) (400 mg, 1.55 mmol) in EtOH (10 ml) and Pd(PPh₃)₂Cl₂ (16.5 mg, 0.023 mmol) were added and stirring was continued for 16 hours under N₂ flow at 50° C. After EtOH was evaporated, EtOAc (25 ml) was added and impurities removed by filtration. The filtrate was washed with saturated aqueous NaHCO₃ (2×20 ml) and EtOAc evaporated. To the residue DCM (20 ml) and water (30 ml) were added and pH was adjusted to 3. The water layer was extracted with DCM (20 ml) at pH 4. The pH of water layer was adjusted to 5.7 and extracted with DCM (2×20 ml). To the combined organic layers (pH 5.7) water was added (20 ml) and pH adjusted to 10.5. The organic layer at pH 10.5 was evaporated to give the foamy crude product which was purified by column chromatography (eluent DCM/MeOH:/NH₃=90:15:1.5) to give the title compound as a foamy white solid (100 mg).

MS (ES+) m/z: [MH₂]²⁺=551.9.

### Example 48

### 4″-O-{3-[3-(5-Carboxy-3,3-dimethyl-6-oxo-1H,6H-2-oxa-3a-aza-phenalen-8-yl)-propoxy]-propyl}-azithromycin

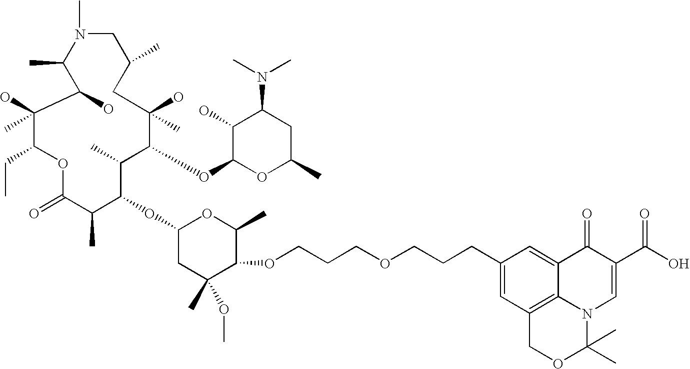

According to the procedure for Example 42 starting from Example 47 4″-O{3-[3-(5-carboxy-3,3-dimethyl-6-oxo-1H,6H-2-oxa-3a-aza-phenalen-8-yl)-prop-2-ynyloxy]-propyl}-azithromycin (100 mg, 0.09 mmol) and 10% Pd/C (20 mg) as catalyst the title compound was obtained as a white solid (60 mg).

MS (ES+) m/z: [MH]⁺=1106.8.

### Example 49

### 4″-O-{3-[4-(5-Carboxy-3,3-dimethyl-6-oxo-1H,6H-2-oxa-3a-aza-phenalen-8-yl)-but-3-ynyloxy]-propyl}-azithromycin

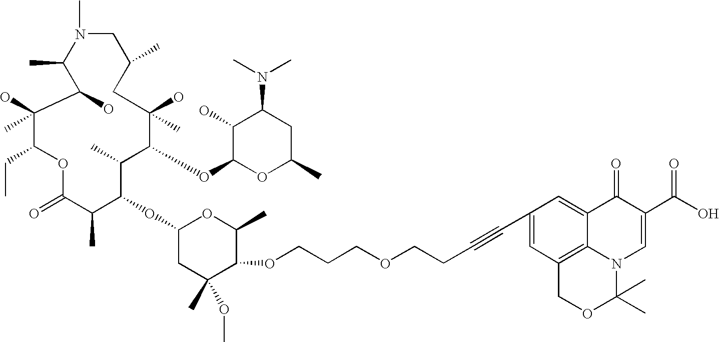

Starting from 4″-O-(3-but-3-ynyloxy-propyl)azithromycin (0.40 g, 0.47 mmol) and 8-iodo-3,3-dimethyl-6-oxo-1H,6H-2-oxa-3a-aza-phenalene-5-carboxylic acid (0.35 g, 0.94 mmol) according to procedure described for Example 47 the title compound was obtained as a white solid (52 mg).

MS (ES+) m/z: [MH]⁺=1116.8.

### Example 50

### 4″-O-{3-[4-(5-Carboxy-3,3-dimethyl-6-oxo-1H,6H-2-oxa-3a-aza-phenalen-8-yl)-butoxy]-propyl}-azithromycin

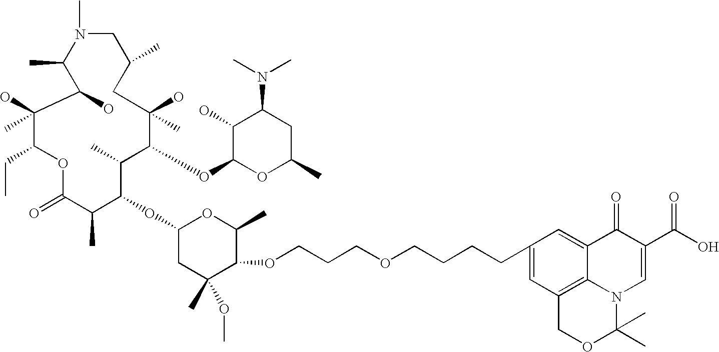

According to the procedure for Example 42 starting from 4″-O-{3-[4-(5-Carboxy-3,3-dimethyl-6-oxo-1H,6H-2-oxa-3a-aza-phenalen-8-yl)-but-3-ynyloxy]-propyl}-azithromycin (52 mg, 0.05 mmol) and 10% Pd/C (10 mg) as catalyst the title compound was obtained as a white solid (23 mg).

MS (ES+) m/z: [MH]⁺=1120.9.

### Examples 51, 52

### 2′-O-Acetyl-4″-O-{2-[3-(3-carboxy-1,4-dihydro-1-(2-methoxyethyl)-4-oxo-6-quinolinyl)prop-2-enyl]-oxyethyl}-azithromycin 11,12-carbonate

### 2′-O-Acetyl-4″-O-{2-[3-(3-carboxy-1,4-dihydro-1-(2-methoxyethyl)-4-oxo-6-quinolinyl)prop-1-enyl]-oxyethyl}-azithromycin 11,12-carbonate

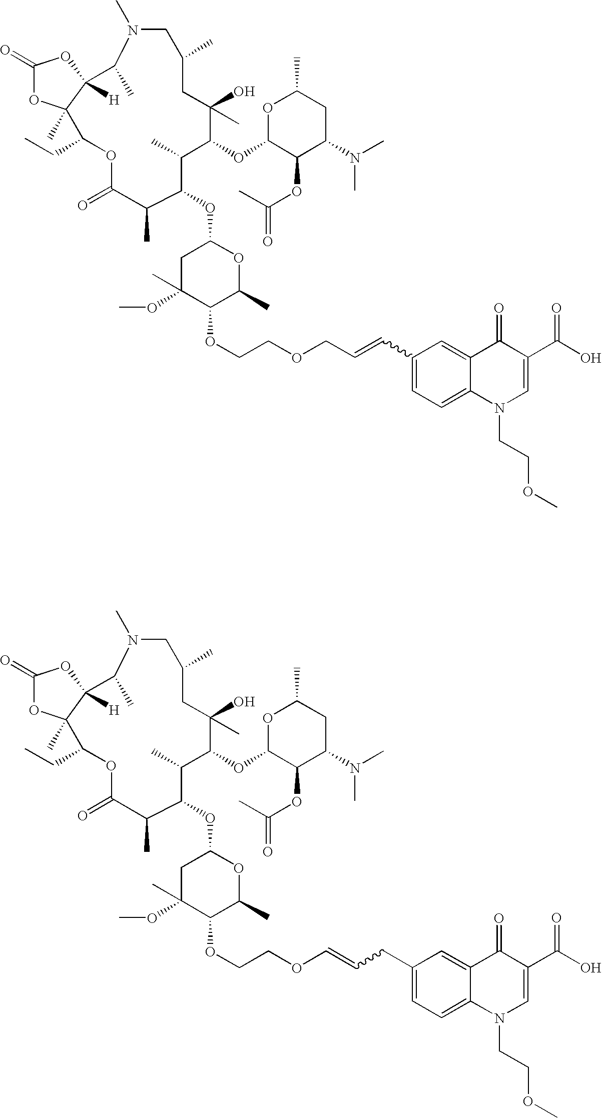

A solution of 2′-O-acetyl-4″-O-(2-allyloxyethyl)-azithromycin 11,12-carbonate (0.29 g, 0.32 mmol) in acetonitrile (6.4 mL) was degassed. Triethylamine (0.64 mL) was added followed by palladium (II) diacetate (0.03 g) and 6-iodo-1(2-methoxyethyl)-4-oxo-1,4-dihydro-quinoline-3-carboxylic acid (0.358 g, 0.96 mmol). The resultant mixture was heated at 80° C. After 2 h a further batch of 6-iodo-1(2-methoxyethyl)-4-oxo-1,4-dihydro-quinoline-3-carboxylic acid (0.109 g) was added and the mixture heated for another 1.5 h. The reaction mixture was then cooled, filtered and purified by chromatography over silica gel eluting with 0-10% (10:1 methanol/0.880 ammonia) in dichloromethane to give the title compounds as a mixture of isomers as a white solid (0.185 g); ESMS m/z 1146.8 [M+H]⁺.

### Example 53

### 2′-O-Acetyl-4″-O-{2-[3-(3-carboxy-1,4-dihydro-1-(2-methoxyethyl)-4-oxo-6-quinolinyl)prop-1-yl]-oxyethyl}-azithromycin 11,12-carbonate

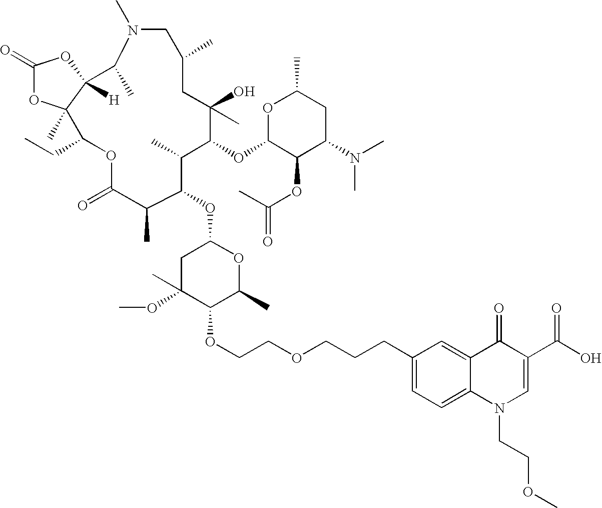

A solution of a mixture of 2′-O-acetyl-4″-O-{2-[3-(3-carboxy-1,4-dihydro-1-(2-methoxyethyl)-4-oxo-6-quinolinyl)prop-2-enyl]-oxyethyl}-azithromycin 11,12-carbonate and 2′-O-Acetyl-4″-O-{2-[3-(3-carboxy-1,4-dihydro-1-(2-methoxyethyl)-4-oxo-6-quinolinyl)prop-1-enyl]-oxyethyl}-azithromycin 11,12-carbonate (0.18 g, 0.15 mmol) in dichloromethane (10 ml) was hydrogenated over 10% palladium charcoal (0.1 g) at atmospheric pressure and room temperature. After 17 h the mixture was filtered and the filtrate purified by chromatography over silica gel eluting with 0-10% (10:1 methanol/0.880 ammonia) in dichloromethane to yield the title compound (0.17 g); ESMS m/z 1148.7 [M+H]⁺.

### Example 54

### 4″-O-{2-[3-(3-Carboxy-1,4-dihydro-1-(2-methoxyethyl)-4-oxo-6-quinolinyl)prop-1-yl]-oxyethyl}-azithromycin

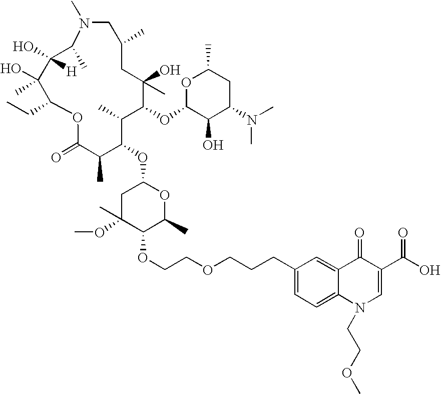

A mixture of acetonitrile (10 mL), 10% aqueous potassium carbonate solution (5 mL) and 2′-O-acetyl-4″-O-{2-[3-(3-carboxy-1,4-dihydro-1-(2-methoxyethyl)-4-oxo-6-quinolinyl)prop-2-enyl]-oxyethyl}-azithromycin 11,12-carbonate (0.17 g, 0.148 mmol) was heated at 80° C. After 9 h the mixture was cooled, partially concentrated and 10% aqueous citric acid added to achieve pH 6. The solution was then extracted with dichloromethane, dried and concentrated. The resultant crude product was purified by chromatography over silica gel eluting with 0-15% (10:1 methanol/0.880 ammonia) in dichloromethane to yield the title compound as a solid (0.085 g); ESMS m/z 540.8 [M+2H]⁺².

### Example 55

### 2′-O-Acetyl-4″-O-{2-[3-(3-carboxy-1,4-dihydro-1-methoxy-4-oxo-6-quinolinyl)prop-2-enyl]-oxyethyl}-azithromycin 11,12-carbonate

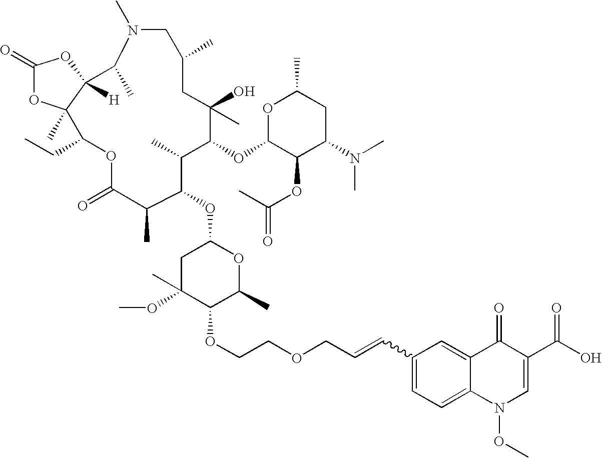

2′-O-Acetyl-4″-O-(2-allyloxyethyl)-azithromycin 11,12-carbonate (0.477 g, 0.53 mmol) was dissolved in degassed acetonitrile (10 ml). Degassed triethylamine (1.05 ml) was added followed by palladium (II) diacetate (0.05 g) and 6-iodo-1-methoxy-4-oxo-1,4-dihydro-quinoline-3-carboxylic acid (0.55 g, 1.59 mmol). The resultant mixture was heated at 80° C. After 1 h a further batch of palladium (II) diacetate (0.03 g) was added and the mixture heated for another 1.5 h. The reaction was then cooled, diluted with dichloromethane, filtered through celite and concentrated. The residue was purified by chromatography over silica gel eluting with 0-15% (10:1 methanol/0.880 ammonia) in dichloromethane to give the title compound and the isomeric compound where the double bond had migrated to be adjacent to the oxygen atom, as a white solid (0.343 g);

ESMS m/z 1118.7 [M+H]⁺.

### Example 56

### 2′-O-Acetyl-4″-O-{2-[3-(3-carboxy-1,4-dihydro-1-methoxy-4-oxo-6-quinolinyl)prop-1-yl]-oxyethyl}-azithromycin 11,12-carbonate

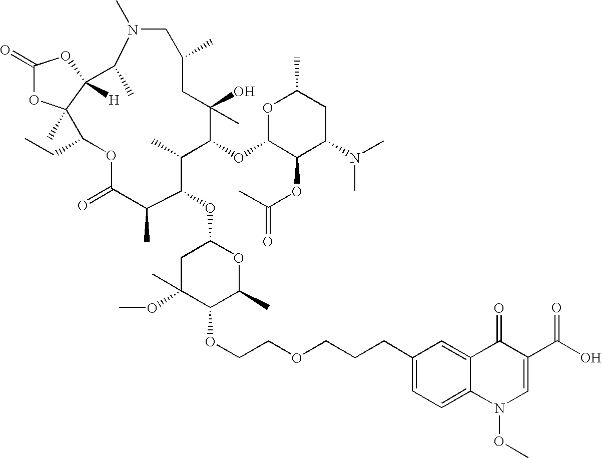

A solution of 2′-O-acetyl-4″-O-{2-[3-(3-carboxy-1,4-dihydro-1-methoxy-4-oxo-6-quinolinyl)prop-2-enyl]-oxyethyl}-azithromycin 11,12-carbonate (0.225 g, 0.2 mmol) in dichloromethane (20 mL) was hydrogenated over 10% palladium charcoal (0.1 g) at atmospheric pressure and room temperature. After 17 h the mixture was filtered and concentrated to yield the title compound (0.19 g); ESMS m/z 1120.7 [M+H]⁺.

### Example 57

### 4″-O-{2-[3-(3-Carboxy-1,4-dihydro-1-methoxy-4-oxo-6-quinolinyl)prop-1-yl]-oxyethyl}-azithromycin

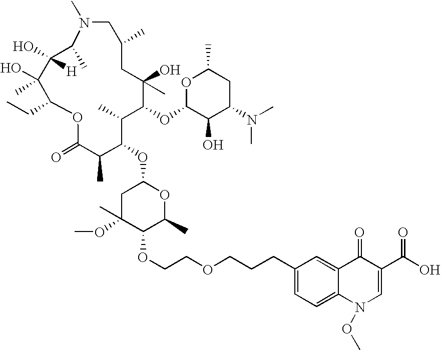

A mixture of acetonitrile (10 mL), 10% aqueous potassium carbonate solution (5 mL) and 2′-O-acetyl-4″-O-{2-[3-(3-carboxy-1,4-dihydro-1-methoxy-4-oxo-6-quinolinyl)prop-2-enyl]-oxyethyl}-azithromycin 11,12-carbonate (0.19 g, 0.17 mmol) was heated at 70° C. After 8 h the mixture was cooled, partially concentrated and 10% aqueous citric acid added to achieve pH 6. The solution was then extracted with dichloromethane and chloroform, dried and concentrated. The resultant crude product was purified by chromatography over silica gel eluting with 0-15% (10:1 methanol/0.880 ammonia) in dichloromethane to yield the title compound as a white solid (0.11 g); ESMS m/z 1052.6 [M+H]⁺.

### Example 58

### 4″-O-{2-[3-(3-carboxy-1,4-dihydro-1-propyl-4-oxo-6-quinolinyl)prop-2-enyl]-oxyethyl}-azithromycin

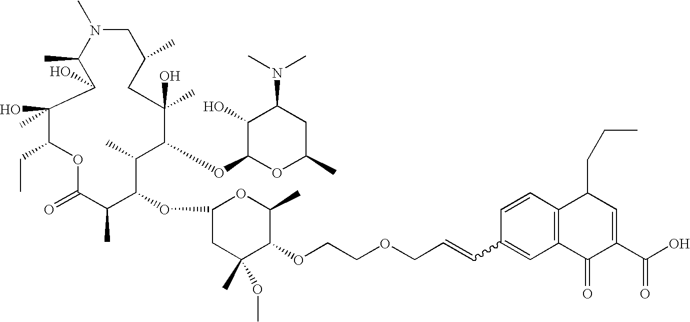

4″-O-(2-Allyloxyethyl)-azithromycin (5.80 g, 6.96 mmol) was dissolved in dimethylacetamide (10 mL) and treated with 6-iodo-1-propyl-4-oxo-1,4-dihydro-quinoline-3-carboxylic acid (1.66 g, 4.64 mmol), tributylamine (0.65 mL), trans-di μ-acetato bis[2-(di-o-tolylphosphino)benzyl]dipalladium (II) (end of spatula) and 2,5 di t-butyl-4-methylphenol (5 crystals). The reaction mixture was stirred under argon at 130° C. for 2 h then allowed to cool down and partitioned between water and dichloromethane. The organic phase was dried (Na₂SO₄) then concentrated and the resulting residue purified by chromatography silica gel eluting with 0-18% (10:1 methanol/0.880 ammonia) in dichloromethane to give the title compound as a white solid (1.79 g); ESMS m/z 531.9 [M+2H]²⁺.

### Example 59

### 4″-O-{2-[3-(3-carboxy-1,4-dihydro-1-propyl-4-oxo-6-quinolinyl)propyl]-oxyethyl}-azithromycin

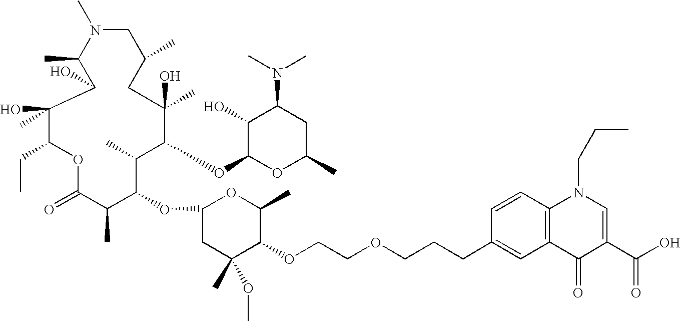

4″-O-{2-[3-(3-Carboxy-1,4-dihydro-1-propyl-4-oxo-6-quinolinyl)prop-1-enyl]-oxyethyl}-azithromycin (1.78 g, 1.68 mmol) in methanol (100 mL) was treated with 10% Pd/C (0.3 g) then hydrogenated at 20° C. and 1 atm for 16 h. The reaction mixture was filtered through celite then concentrated and the resulting residue purified by chromatography eluting with 0-18% (10:1 methanol/0.880 ammonia) in dichloromethane to give the title compound as a white solid (1.13 g); ESMS m/z 532.9 [M+2H]²⁺.

### Examples 60, 61

### 4″-O-{2-[3-(3-carboxy-1,4-dihydro-1-methyl-4-oxo-6-quinolinyl)prop-2-enyl]-oxyethyl}-azithromycin

### 4″-O-{2-[3-(3-carboxy-1,4-dihydro-1-methyl-4-oxo-6-quinolinyl)prop-1-enyl]-oxyethyl}-azithromycin

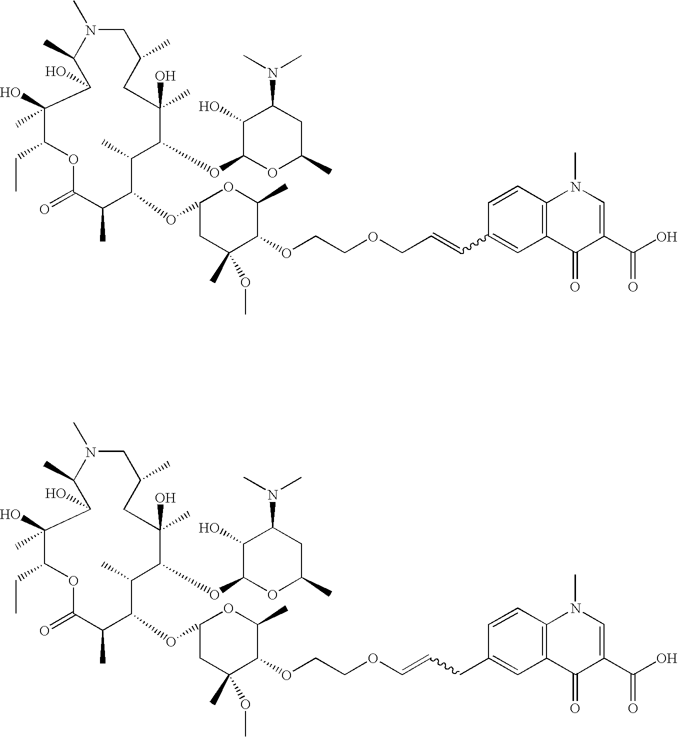

4″-O-(2-Allyloxyethyl)-azithromycin (0.38 g, 0.45 mmol) was dissolved in dimethylacetamide (0.9 mL) and treated with 6-iodo-1-methyl-4-oxo-1,4-dihydro-quinoline-3-carboxylic acid (0.1 g, 0.3 mmol), tributylamine (0.043 ml), trans-di μ-acetato bis[2-(di-o-tolylphosphino)benzyl]dipalladium (II) (end of spatula) and 2,5 di t-butyl-4-methylphenol (1 crystal). The reaction mixture was stirred under argon at 115° C. for 17 h then concentrated and the resulting residue purified by chromatography over silica gel eluting with 0-15% (10:1 methanol/0.880 ammonia) in dichloromethane to give the title compounds as a mixture of isomers as a white solid (0.164 g); ESMS m/z 1034.7 [M+H]⁺.

### Example 62

### 4″-O-{2-[3-(3-Carboxy-1,4-dihydro-1-methyl-4-oxo-6-quinolinyl)propyl]-oxyethyl}-azithromycin

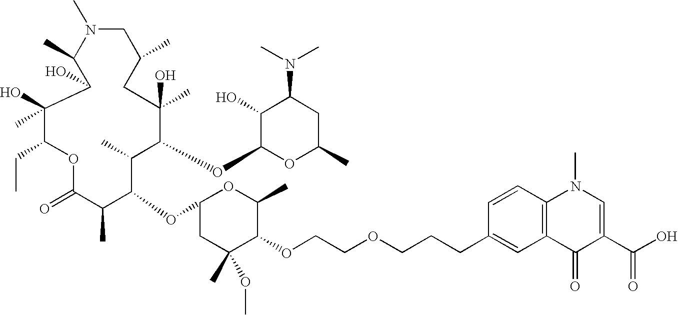

A mixture of 4″-O-{2-[3-(3-Carboxy-1,4-dihydro-1-methyl-4-oxo-6-quinolinyl)prop-2-enyl]-oxyethyl}-azithromycin and 4″-O-{2-[3-(3-carboxy-1,4-dihydro-1-methyl-4-oxo-6-quinolinyl)prop-1-enyl]-oxyethyl}-azithromycin (0.164 g, 0.158 mmol) in methanol (50 mL) was treated with 10% Pd/C (0.07 g). After two mins the catalyst was filtered off and replaced with a fresh portion of 10% Pd/C (0.07 g) then hydrogenated at 20° C. and 1 atm for 70 h. The reaction mixture was filtered then concentrated to give the title compound as a pale yellow solid (0.158 g); ESMS m/z 1036.8 [M+H]⁺.

### Examples 63, 64

### 4″-O-{2-[3-(3-Carboxy-1,4-dihydro-4-oxo-1-iso-propyl-6-quinolinyl)prop-2-enyl]-oxyethyl}-azithromycin

### 4″-O-{2-[3-(3-Carboxy-1,4-dihydro-4-oxo-1-iso-propyl-6-quinolinyl)prop-1-enyl]-oxyethyl}-azithromycin

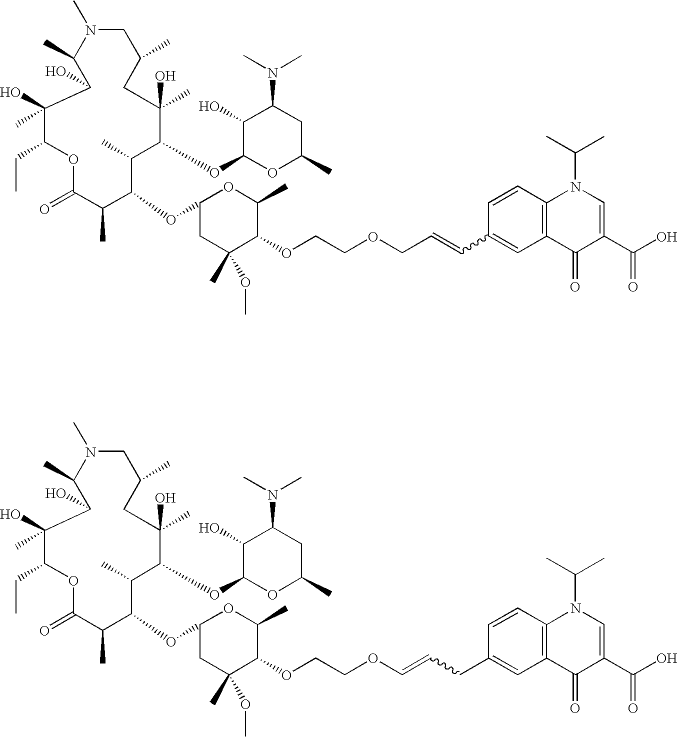

4″-O-(2-Allyloxyethyl)-azithromycin (0.38 g, 0.45 mmol) was dissolved in dimethylacetamide (0.9 mL) and treated with 6-iodo-4-oxo-1-iso-propyl-1,4-dihydro-quinoline-3-carboxylic acid (0.108 g, 0.3 mmol), tributylamine (0.043 mL), trans-di μ-acetato bis[2-(di-o-tolylphosphino)benzyl]dipalladium (II) (end of spatula) and 2,5 di t-butyl-4-methylphenol (1 crystal). The reaction mixture was stirred under argon at 100° C. for 2 h then at 105° C. for 17 h. Ethyl acetate (20 mL) was added and the solution extracted (3×20 mL) with an aqueous saturated solution of sodium bicarbonate. The organic phase was treated with an aqueous solution of acetic acid (pH˜5). The pH of the aqueous phase was increased to −9 by the addition of aqueous ammonia. The resulting aqueous phase was extracted with ethyl acetate (2×40 mL), the combined organic phases were dried (K₂CO₃) and concentrated. The resulting residue was purified by chromatography over silica gel eluting with 0-10% (10:1 methanol/0.880 ammonia) in dichloromethane to give the title compounds as a mixture of isomers as a white solid (0.2 g); ESMS m/z 531.8 [M+2H]²⁺.

### Example 65

### 4″-O-{2-[3-(3-Carboxy-1,4-dihydro-4-oxo-1-iso-propyl-6-quinolinyl)propyl]-oxyethyl}-azithromycin

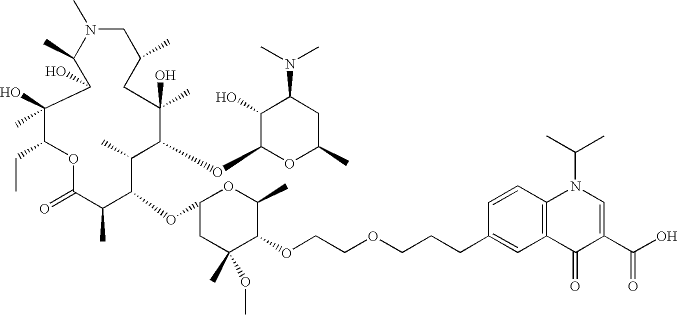

A mixture of 4″-O-{2-[3-(3-Carboxy-1,4-dihydro-4-oxo-1-iso-propyl-6-quinolinyl)prop-2-enyl]-oxyethyl}-azithromycin and 4″-O-{2-[3-(3-Carboxy-1,4-dihydro-4-oxo-1-iso-propyl-6-quinolinyl)prop-1-enyl]-oxyethyl}-azithromycin (0.2 g, 0.19 mmol) in methanol (40 mL) was treated with 10% Pd/C (0.06 g). After two mins the catalyst was filtered off and replaced with a fresh portion of 10% Pd/C (0.06 g) then hydrogenated at 20° C. and 1 atm for 17 h. The reaction mixture was filtered then concentrated to give the title compound as a white solid (0.18 g); ESMS m/z 532.8 [M+2H]²⁺.

### Examples 66, 67

### 4″-O{2-[3-(3-Carboxy-1,4-dihydro-4-oxo-1-cyclopropyl-6-quinolinyl)prop-2-enyl]-oxyethyl}-azithromycin

### 4″-O{2-[3-(3-Carboxy-1,4-dihydro-4-oxo-1-cyclopropyl-6-quinolinyl)prop-1-enyl]-oxyethyl}-azithromycin

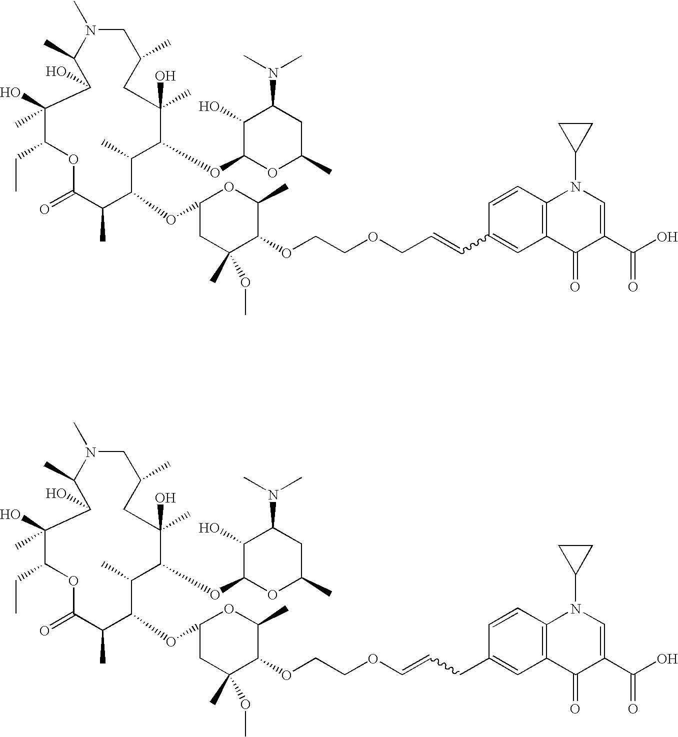

4″-O-(2-Allyloxyethyl)-azithromycin (0.318 g, 0.377 mmol) was dissolved in dimethylacetamide (0.75 mL) and treated with 6-iodo-4-oxo-1-cyclopropyl-1,4-dihydro-quinoline-3-carboxylic acid (0.90 g, 0.25 mmol), tributylamine (0.036 mL), trans-di μ-acetato bis[2-(di-o-tolylphosphino)benzyl]dipalladium (II) (end of spatula) and 2,5 di t-butyl-4-methylphenol (1 crystal). The reaction mixture was stirred under argon at 100° C. for 8 h. Dichloromethane and water were added. The aqueous phase was extracted with dichloromethane then the combined organic phases were dried (Na₂SO₄) and concentrated. The resulting residue was purified by chromatography over silica gel eluting with 0-10% (10:1 methanol/0.880 ammonia) in dichloromethane to give the title compound and the isomeric compound where the double bond had migrated to be adjacent to the oxygen atom, as a white solid (0.24 g); ESMS m/z 530.8 [M+2H]²⁺.

### Example 68

### 4″-O-{2-[3-(3-Carboxy-1,4-dihydro-1-cyclopropyl-4-oxo-6-quinolinyl)propyl]-oxyethyl}-azithromycin

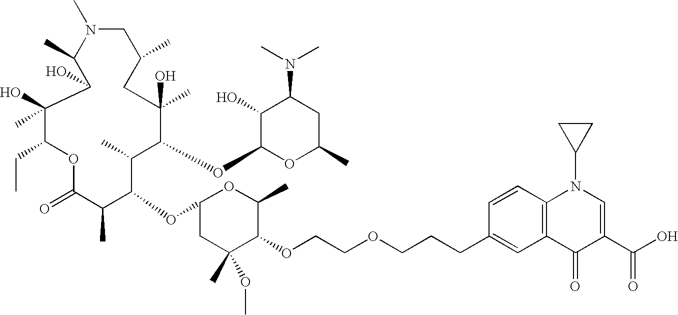

A mixture of 4″-O-{2-[3-(3-carboxy-1,4-dihydro-4-oxo-1-cyclopropyl-6-quinolinyl)prop-2-enyl]-oxyethyl}-azithromycin and 4″-O-{2-[3-(3-carboxy-1,4-dihydro-4-oxo-1-cyclopropyl-6-quinolinyl)prop-1-enyl]-oxyethyl}-azithromycin (0.24 g, 0.22 mmol) in methanol (80 mL) was treated with 10% Pd/C (0.08 g). After two mins the catalyst was filtered off and replaced with a fresh portion of 10% Pd/C (0.06 g) then hydrogenated at 20° C. and 1 atm for 17 h. The reaction mixture was filtered then concentrated to give the title compound as a white solid (0.195 g); ESMS m/z 531.7 [M+2H]²⁺.

### Examples 69, 70

### 4″-O-{2-[3-(2-Carboxy-6,7-dihydro-1H,5H-pyrido[3,2,1-ij]-1-oxo-9-quinolinyl)prop-2-enyl]-oxyethyl}-azithromycin

### 4″-O{2-[3-(2-Carboxy-6,7-dihydro-1H,5H-pyrido[3,2,1-ij]-1-oxo-9-quinolinyl)prop-1-enyl]-oxyethyl}-azithromycin

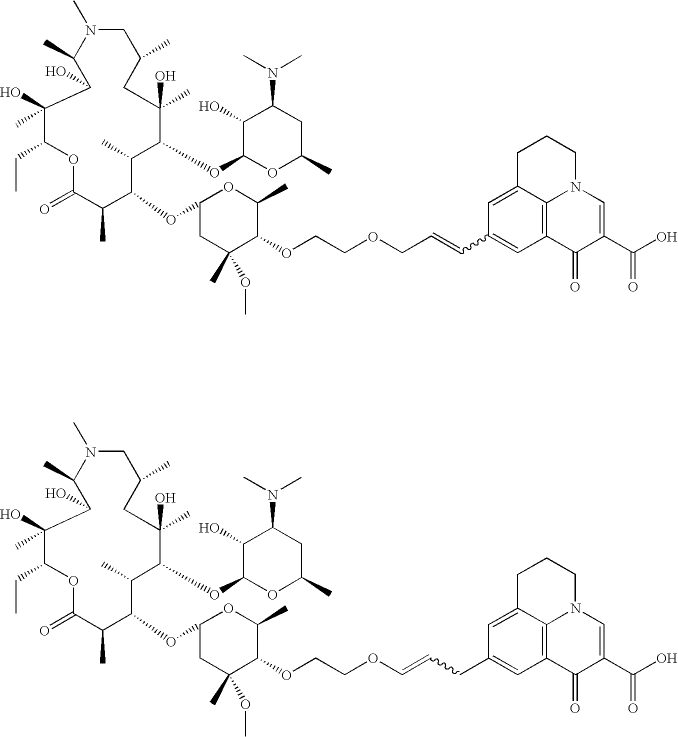

4″-O-(2-Allyloxyethyl)-azithromycin (0.424 g, 0.5 mmol) was dissolved in dimethylacetamide (1 mL) and treated with 9-bromo-1-oxo-6,7-dihydro-1H,5H-pyrido[3,2,1-ij]quinoline-2-carboxylic acid (0.104 g, 0.33 mmol), tributylamine (0.048 mL), trans-di μ-acetato bis[2-(di-o-tolylphosphino)benzyl]dipalladium (II) (end of spatula) and 2,5 di t-butyl-4-methylphenol (1 crystal). The reaction mixture was stirred under argon at 130° C. for 17 h. Dichloromethane and water were added. The aqueous phase was extracted with dichloromethane then the combined organic phases were dried (Na₂SO₄) and concentrated. The resulting residue was purified by chromatography silica gel eluting with 0-10% (10:1 methanol/0.880 ammonia) in dichloromethane to give the title compounds as an isomeric mixture as a white solid (0.2 g); ESMS m/z 530.9 [M+2H]²⁺.

### Example 71

### 4″-O-{2-[3-(2-Carboxy-6,7-dihydro-1H,5H-pyrido[3,2,1-ij]-1-oxo-9-quinolinyl)propyl]-oxyethyl}-azithromycin

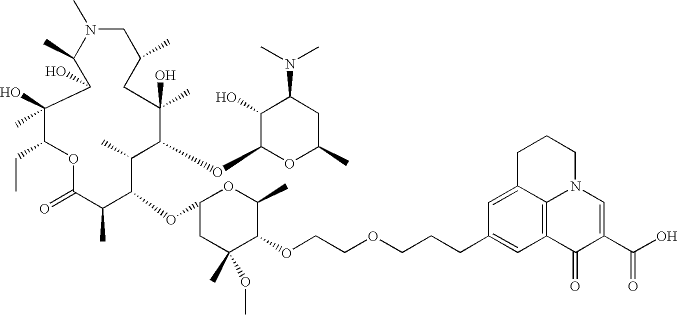

A mixture of 4″-O-{2-[3-(2-carboxy-6,7-dihydro-1H,5H-pyrido[3,2,1-ij]-1-oxo-9-quinolinyl)prop-2-enyl]-oxyethyl}-azithromycin and 4″-O-{2-[3-(2-carboxy-6,7-dihydro-1H,5H-pyrido[3,2,1-ij]-1-oxo-9-quinolinyl)prop-1-enyl]-oxyethyl}-azithromycin (0.2 g, 0.18 mmol) in methanol (40 mL) was treated with 10% Pd/C (0.07 g). After two mins the catalyst was filtered off and replaced with a fresh portion of 10% Pd/C (0.07 g) then hydrogenated at 20° C. and 1 atm for 17 h. The reaction mixture was filtered then concentrated to give the title compound as a white solid (0.17 g); ESMS m/z 531.8 [M+2H]²⁺.

### Examples 72, 73

### 4″-O-{2-[3-(6-Carboxy-7-oxo-2,3-dihydro-7H-[1,4]oxazino[2,3,4-ij]quinolin-9-yl) prop-2-enyl]-oxyethyl}-azithromycin

### 4″-O-{2-[3-(6-Carboxy-7-oxo-2,3-dihydro-7H-[1,4]oxazino[2,3,4-ij]quinolin-9-yl) prop-1-enyl]-oxyethyl}-azithromycin

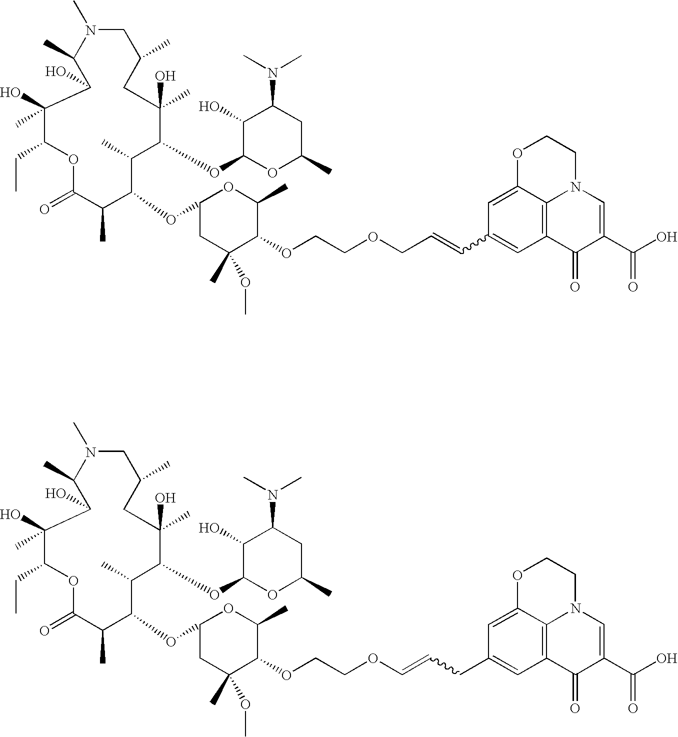

4″-O-(2-Allyloxyethyl)-azithromycin (0.38 g, 0.45 mmol) was dissolved in dimethylacetamide (0.9 mL) and treated with 9-iodo-7-oxo-2,3-dihydro-7H-[1,4]oxazino[2,3,4-ij]quinoline-6-carboxylic acid (0.107 g, 0.3 mmol), tributylamine (0.043 mL), trans-di μ-acetato bis[2-(di-o-tolylphosphino)-benzyl]dipalladium (II) (end of spatula) and 2,5 di t-butyl-4-methylphenol (1 crystal). The reaction mixture was stirred under argon at 110° C. for 17 h, cooled down then concentrated and the resulting residue purified by chromatography silica gel eluting with 0-15% (10:1 methanol/0.880 ammonia) in dichloromethane to give a mixture of the title compounds as a white solid (0.22 g);

ESMS m/z 1062.6 [M+H]⁺.

### Example 74

### 4″-O-{2-[3-(6-Carboxy-7-oxo-2,3-dihydro-7H-[1,4]oxazino[2,3,4-ij]quinolin-9-yl) propyl]-oxyethyl}-azithromycin

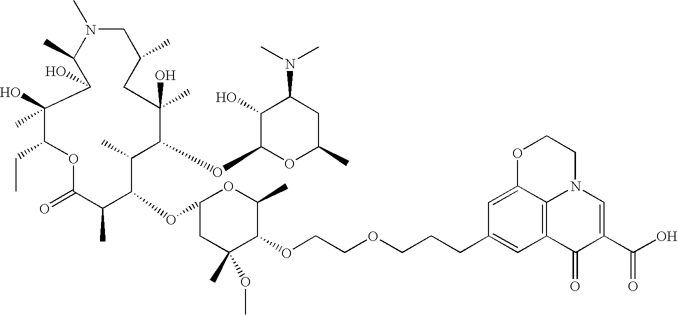

A mixture of 4″-O-{2-[3-(6-Carboxy-7-oxo-2,3-dihydro-7H-[1,4]oxazino[2,3,4-ij]quinolin-9-yl)prop-1-enyl]-oxyethyl}-azithromycin and 4″-O-{2-[3-(6-carboxy-7-oxo-2,3-dihydro-7H-[1,4]oxazino[2,3,4-ij]quinolin-9-yl)prop-2-enyl]-oxyethyl}-azithromycin (0.22 g, 0.2 mmol) in methanol (70 mL) was treated with 10% Pd/C (0.1 g) then hydrogenated at 20° C. and 1 atm for 70 h. The reaction mixture was filtered then concentrated to give the title compound as a white solid (0.19 g); ESMS m/z 532.9 [M+2H]²⁺.

### Examples 75, 76

### 4″-O{2-[3-(6-Carboxy-3,3-dimethyl-7-oxo-2,3-dihydro-7H-[1,4]oxazino[2,3,4-ij]quinolin-9-yl)prop-2-enyl]-oxyethyl}-azithromycin

### 4″-O{2-[3-(6-Carboxy-3,3-dimethyl-7-oxo-2,3-dihydro-7H-[1,4]oxazino[2,3,4-ij]quinolin-9-yl)prop-1-enyl]-oxyethyl}-azithromycin

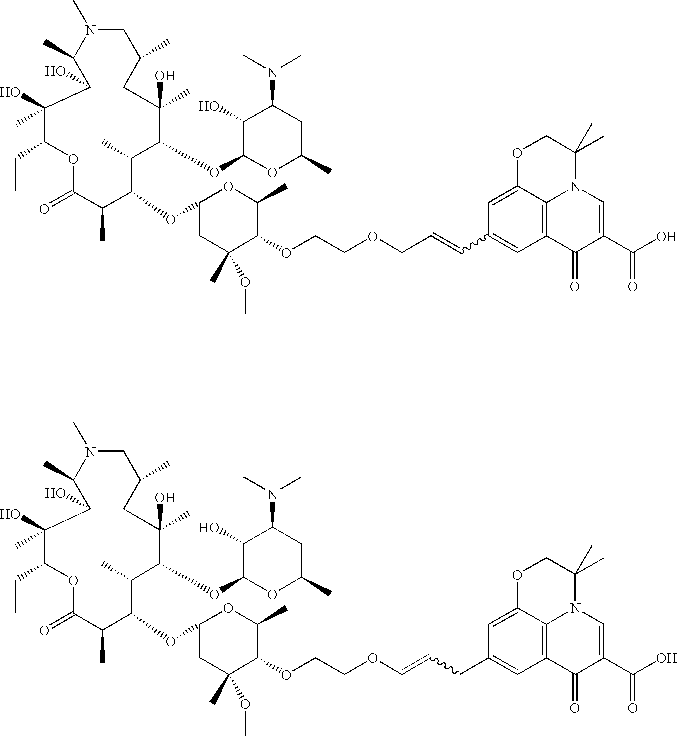

4″-O-(2-Allyloxyethyl)-azithromycin (0.38 g, 0.45 mmol) was dissolved in dimethylacetamide (0.9 mL) and treated with 9-iodo-3,3-dimethyl-7-oxo-2,3-dihydro-7H-[1,4]oxazino[2,3,4-ij]quinoline-6-carboxylic acid (0.115 g, 0.3 mmol), tributylamine (0.043 mL), trans-di μ-acetato bis[2-(di-o-tolylphosphino)benzyl]dipalladium (II) (end of spatula) and 2,5 di t-butyl-4-methylphenol (1 crystal). The reaction mixture was stirred under argon at 115° C. for 17 h, cooled down then concentrated and the resulting residue purified by chromatography over silica gel eluting with 0-15% (10:1 methanol/0.880 ammonia) in dichloromethane to give a mixture of the title compounds, as a white solid (0.23 g); ESMS m/z 545.8 [M+2H]²⁺.

### Example 77

### 4″-O-{2-[3-(6-Carboxy-3,3-dimethyl-7-oxo-2,3-dihydro-7H-[1,4]oxazino[2,3,4-ij]quinolin-9-yl)propyl]-oxyethyl}-azithromycin

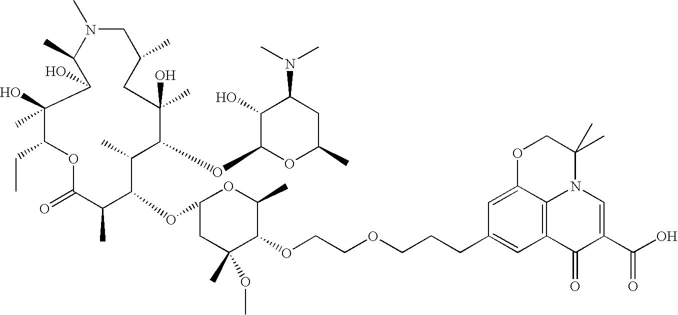

A mixture of 4″-O-{2-[3-(6-carboxy-3,3-dimethyl-7-oxo-2,3-dihydro-7H-[1,4]oxazino[2,3,4-ij]quinolin-9-yl)prop-2-enyl]-oxyethyl}-azithromycin and 4″-O{2-[3-(6-carboxy-3,3-dimethyl-7-oxo-2,3-dihydro-7H-[1,4]oxazino[2,3,4-ij]quinolin-9-yl)prop-1-enyl]-oxyethyl}-azithromycin (0.23 g, 0.21 mmol) in methanol (70 mL) was treated with 10% Pd/C (0.1 g) then hydrogenated at 20° C. and 1 atm for 17 h. The reaction mixture was filtered concentrated and the resulting residue purified by chromatography silica gel eluting with 0-15% (10:1 methanol/0.880 ammonia) in dichloromethane to give the title compound as a white solid (0.15 g); ESMS m/z 546.9 [M+2H]²⁺.

### Examples 78, 79

### 4″-O{2-[3-(3-Carboxy-1,4-dihydro-4-oxo-1-cyclo-propylmethyl-6-quinolinyl)prop-2-enyl]-oxyethyl}-azithromycin

### 4″-O{2-[3-(3-Carboxy-1,4-dihydro-4-oxo-1-cyclo-propylmethyl-6-quinolinyl)prop-1-enyl]-oxyethyl}-azithromycin

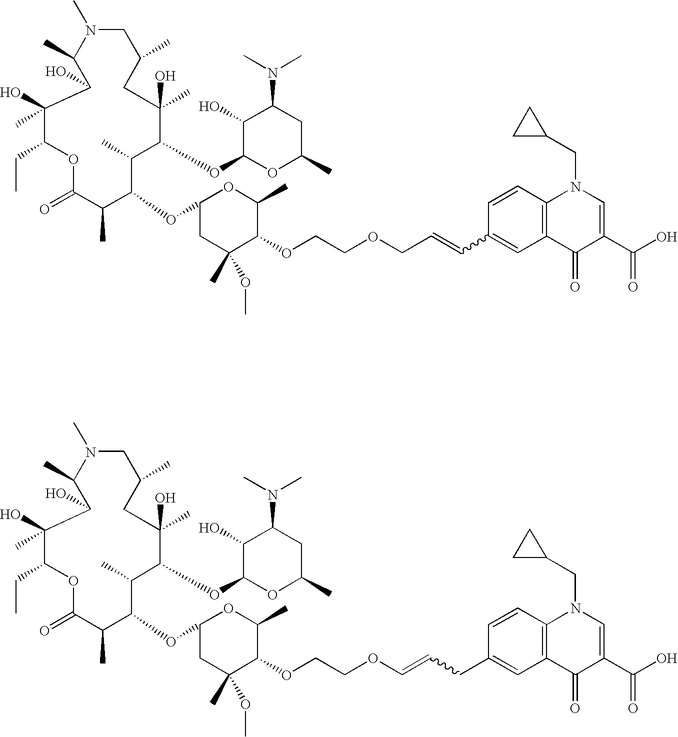

6-Iodo-4-oxo-1-cyclo-propylmethyl-1,4-dihydro-quinoline-3-carboxylic acid (0.443 g), tributylamine (0.175 ml) trans-di μ-acetato bis[2-(di-o-tolylphosphino)benzyl]dipalladium (II) (˜10 mg) and 2,5 di t-butyl-4-methylphenol (˜10 mg) dissolved in dimethylacetamide (10 ml) was de-oxygenated by stirring at 40° C. while a gentle stream of argon was bubbled through for 20 min. The argon stream was stopped, 4″-O-(2-allyloxyethyl)-azithromycin (1.0 g) added and the temperature increased to 125° C. After 16 h the mixture was cooled and water (30 mL) added to form a light brown precipitate which was dried under vacuum (4 mbar) overnight (0.3 g). The aqueous layer was extracted with DCM (×2) and the organic extracts dried and evaporated to yield a brown syrup. After standing under vacuum (4 mbar) overnight a glass had formed which was partitioned between ethyl acetate and water. The layers were separated and the aqueous phase extracted with fresh portion of ethyl acetate. The combined organic layers were then washed with water (×2), dried and evaporated to yield a beige solid (0.3 g). The two samples of crude product thus obtained were combined and purified by chromatography over silica gel eluting with 0-15% (10:1 methanol/0.880 ammonia) in dichloromethane to give a mixture of the title compounds as a white solid (0.163 g); ESMS m/z 537.9 [M+2H]²⁺.

### Example 80

### 4″-O-{2-[3-(3-Carboxy-1,4-dihydro-1-cyclo-propylmethyl-4-oxo-6-quinolinyl)propyl]-oxyethyl}-azithromycin

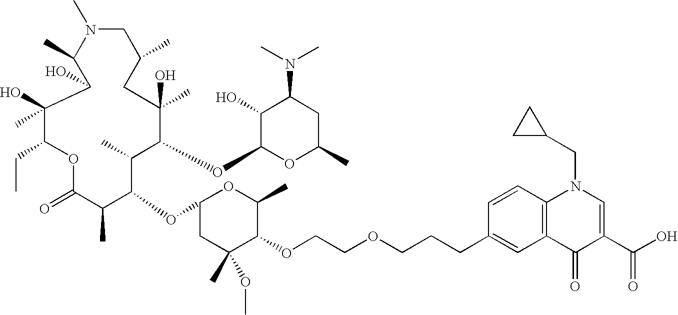

A mixture of isomers (4″-O-{2-[3-(3-Carboxy-1,4-dihydro-4-oxo-1-cyclo-propylmethyl-6-quinolinyl)prop-1-enyl]-oxyethyl}-azithromycin and 4″-O-{2-[3-(3-carboxy-1,4-dihydro-4-oxo-1-cyclo-propylmethyl-6-quinolinyl)prop-2-enyl]-oxyethyl}-azithromycin) (0.163 g) in methanol (10 ml) was treated with 10% Pd/C (˜0.1 g) and the resultant mixture hydrogenated at 20° C. and 1 atm for 48 h. The reaction mixture was filtered through then concentrated to give a yellow foam. The foam was dissolved in DCM (2 mL), treated with charcoal, filtered and evaporated to yield the title compound as a beige solid (0.937 g); ESMS m/z 1074.8 [M−H]⁻.

### Example 81

### 4″-O-{2-[3-(3-carboxy-1,4-dihydro-1-butyl-4-oxo-6-quinolinyl)prop-2-enyl]-oxyethyl}-azithromycin ethyl ester

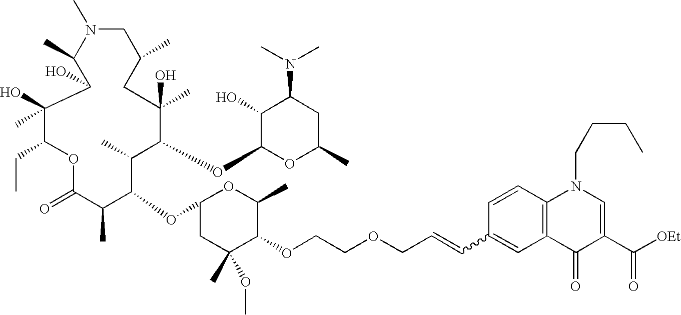

4″-O-(2-Allyloxyethyl)-azithromycin (0.70 g, 0.840 mmol) was dissolved in dimethylacetamide (3 mL) and treated with ethyl 1-butyl-6-iodo-4-oxo-1,4-dihydro-3-quinolinecarboxylate (0.34 g, 0.840 mmol), tributylamine (0.12 mL), trans-di μ-acetato bis[2-(di-o-tolylphosphino)benzyl]dipalladium (II) (1 crystal) and 2,5 di t-butyl-4-methylphenol (1 crystal). The reaction mixture was stirred under argon at 125° C. for 22 h then allowed to cool down and partitioned between water and dichloromethane(3×). The organic phase were combined and dried (MgSO₄) then concentrated and the resulting residue purified by silica gel chromatography eluting with 0-12% (10:1 methanol/0.880 ammonia) in dichloromethane to give the title compound as a white solid (0.71 g); ESMS m/z 552.9 [M+2H]²⁺.

### Example 82

### 4″-O-{2-[3-(3-carboxy-1,4-dihydro-1-butyl-4-oxo-6-quinolinyl)propyl]-oxyethyl}-azithromycin ethyl ester

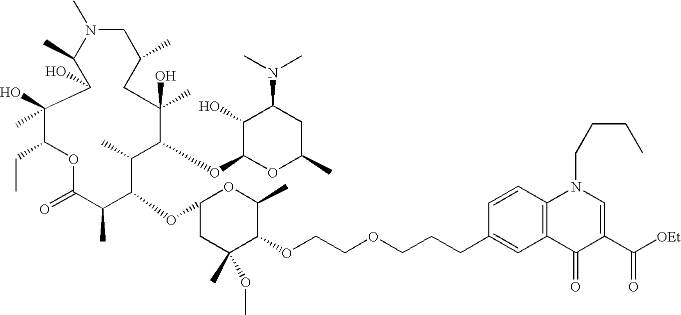

4″-O-{2-[3-(3-carboxy-1,4-dihydro-1-butyl-4-oxo-6-quinolinyl)prop-2-enyl]-oxyethyl}-azithromycin ethyl ester (0.70 g, 0.634 mmol) in methanol (30 mL) was treated with 10% Pd/C (0.8 g) then hydrogenated at 1 atm for 16 h. The reaction mixture was filtered through celite then concentrated and the resulting residue purified by silica gel chromatography eluting with 0-12% (10:1 methanol/0.880 ammonia) in dichloromethane to give the title compound as a pale yellow solid (0.50 g); ESMS m/z 553.9 [M+2H]²⁺.

### Example 83

### 4″-O-{2-[3-(3-carboxy-1,4-dihydro-1-butyl-4-oxo-6-quinolinyl)propyl]-oxyethyl}-azithromycin

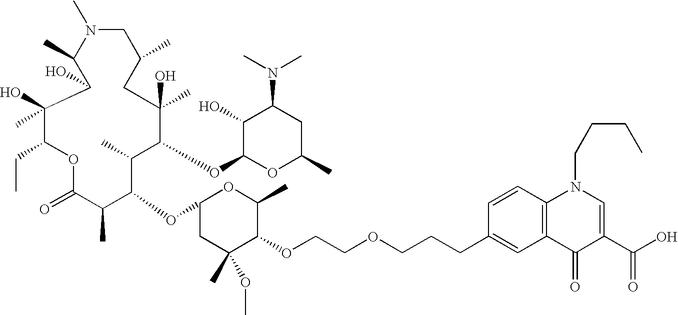

4″-O-{2-[3-(3-carboxy-1,4-dihydro-1-butyl-4-oxo-6-quinolinyl)propyl]-oxyethyl}-azithromycin ethyl ester (0.49 g, 0.443 mmol) was dissolved in THF (10 mL) and treated with 0.5M aqueous lithium hydroxide (1.8 mL, 0.9 mmol) then stirred at 20° C. for 16 h. 2M aqueous acetic acid (0.5 mL, 1.0 mmol) was added and the mixture concentrated. The resulting residue was purified by silica gel chromatography eluting with 0-18% (10:1 methanol/0.880 ammonia) in dichloromethane to give the title compound as a white solid (0.44 g); ESMS m/z 539.9 [M+2H]²⁺

### Example 84

### 2′-O-Acetyl-4″-O-{2-[3-(3-carboxy-1,4-dihydro-1-ethyl-4-oxo-6-quinolinyl)prop-1-enyl]-oxyethyl}-azithromycin 11,12-carbonate

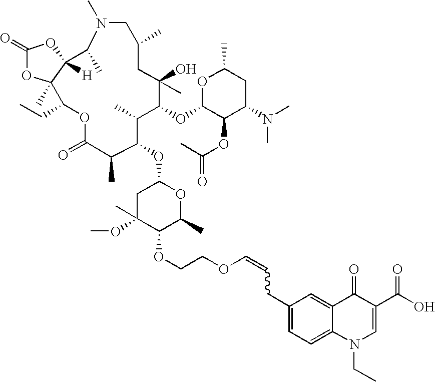

A solution of 2′-O-acetyl-4″-O-(2-allyloxyethyl)-azithromycin 11,12-carbonate (0.29 g, 0.32 mmol) in acetonitrile (6.4 mL) was degassed. Degassed triethylamine (0.64 mL) was added followed by palladium (II) diacetate (0.03 g) and 6-iodo-1-ethyl-4-oxo-1,4-dihydro-quinoline-3-carboxylic acid (0.329 g, 0.96 mmol). The resultant mixture was heated at 80° C. After 1.75 h a further batch of 6-iodo-1-ethyl-4-oxo-1,4-dihydro-quinoline-3-carboxylic acid (0.109 g) was added and the mixture heated for another 1.25 h. The reaction was then cooled, filtered, concentrated and purified by chromatography over silica gel eluting with 0-10% (10:1 methanol/0.880 ammonia) in dichloromethane to give the title compound (cis:trans=90:10) as a white solid (0.14 g); ESMS m/z 558.8 [M+2H]²⁺.

### Example 85

### 4″-O-{2-[3-(3-Carboxy-1,4-dihydro-1-(2-methoxyethyl)-4-oxo-6-quinolinyl)prop-1-enyl]-oxyethyl}-azithromycin

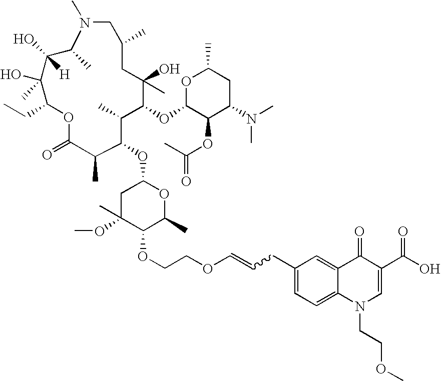

4″-O-(2-Allyloxyethyl)-azithromycin (0.23 g, 0.276 mmol) was dissolved in dimethylacetamide (0.56 ml) and treated with 6-iodo-1(2-methoxyethyl)-4-oxo-1,4-dihydro-quinoline-3-carboxylic acid (0.067 g, 0.18 mmol), tributylamine (0.026 ml), trans-di μ-acetato bis[2-(di-o-tolylphosphino)benzyl]dipalladium (II) (end of spatula) and 2,5 di t-butyl-4-methylphenol (1 crystal). The reaction mixture was stirred under argon at 115° C. for 17 h, cooled down then concentrated and the resulting residue purified by chromatography silica gel eluting with 0-15% (10:1 methanol/0.880 ammonia) in dichloromethane to give the title compound (trans:cis=80:20) as a white solid (0.1 g);

ESMS m/z 539.9 [M+2H]²⁺.

### Example 86

### 2′-O-Acetyl-4″-O-{2-[2-(3-carboxy-1,4-dihydro-1-ethyl-4-oxo-6-quinolinyl)-1-propenyl]-oxyethyl}-azithromycin 11,12-carbonate ethyl ester

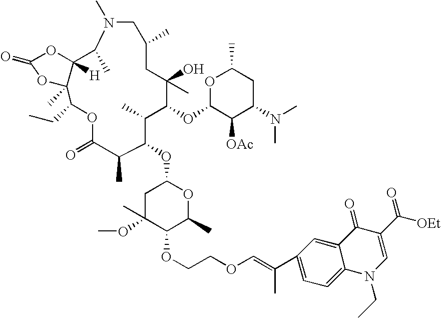

A solution of triethylamine (9.9 mL) in dry acetonitrile (100 mL) was degassed by bubbling argon while ultrasonicating for 5 min. To this solution was added ethyl 1-ethyl-6-iodo-4-oxo-1,4-dihydro-3-quinolinecarboxylate (5.27 g), triphenyl phosphine (0.123 g) and sodium bicarbonate (1.2 g). A degassed solution of 2′-O-acetyl-4″-O-(2-allyloxyethyl)-azithromycin 11,12-carbonate (6.4 g) in dry acetonitrile (100 mL) was added to the above solution followed by palladium diacetate (0.377 g). The reaction was refluxed for 2 h after which further palladium acetate (0.377 g) was added. The reaction was refluxed for a further 4 h, cooled, filtered through celite and evaporated under reduced pressure to dryness to give the crude product which was partially purified by chromatography over silica gel eluting with 0-8% (10:1 methanol/0.880 ammonia) in dichloromethane to yield the title compound in a mixture with compounds from example 19 (11 g); ESMS m/z 572.9 [M+2H]⁺⁺.

### Example 87

### 4″-O-{2-[2-(3-carboxy-1,4-dihydro-1-ethyl-4-oxo-6-quinolinyl)-prop-1-enyl]-oxyethyl}-azithromycin

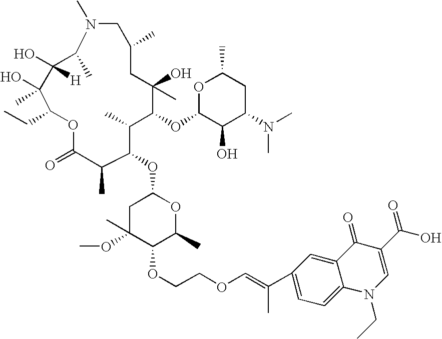

To a solution of 2′-O-acetyl-4″-O-{2-[2-(3-carboxy-1,4-dihydro-1-ethyl-4-oxo-6-quinolinyl)-1-propenyl]-oxyethyl}-azithromycin 11,12-carbonate ethyl ester (11 g) in dichloromethane (400 mL) was added 10% Pd/C (5 g). After 5 min the catalyst was filtered through celite and replaced with fresh 10% Pd/C (5 g) then hydrogenated at 20° C. and 1 atm for 67 h. The reaction was filtered through celite and concentrated. The residue was dissolved in acetonitrile (300 mL) and treated with 10% aqueous solution of potassium carbonate (150 mL). The reaction mixture was heated at 80° C. for 48 h then methanol (100 mL) was added and the reaction mixture heated for a further 24 h. The mixture was cooled down, the organic phase was separated then partially concentrated. The pH of both phases was adjusted to 6 by adding a 10% aqueous solution of citric acid followed by a dichloromethane extraction. The combined organic phases were dried (Na₂SO₄), concentrated and the residue chromatographed over silica gel eluting with 0-14% (10:1 methanol/0.880 ammonia) in dichloromethane. Further purifications over silica gel with 0-9% (10:1 methanol/0.880 ammonia) in dichloromethane followed by preparative reverse phase HPLC (MeCN/H₂O/0.1% HCO₂H eluent) and finally chromatography over silica gel eluting with 0-15% (10:1 methanol/0.880 ammonia) in dichloromethane gave the title compound as a white solid (0.033 g); ESMS m/z 524.7 [M+2H]⁺⁺.

### Example 88

### 4″-O-{3-[3-(3-Carboxy-1-ethyl-4-oxo-1,4-dihydro-quinolin-6-yl)-prop-2-ynyloxy]-propyl}-azithromycin

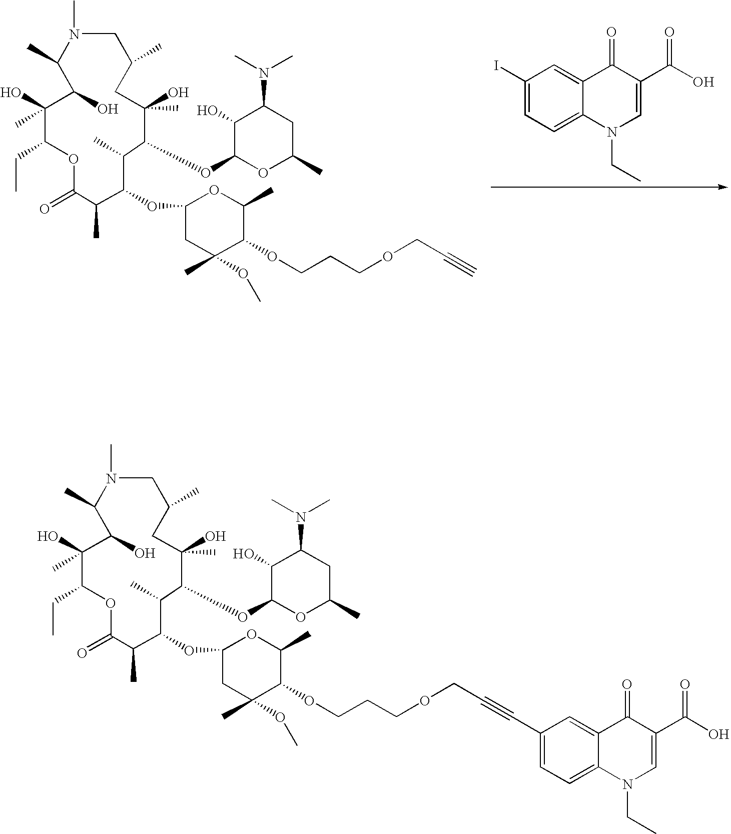

4″-O-[3-(Prop-2-ynyloxy)-propyl]-azithromycin (9.6 g, 7.3 mmol) was dissolved in MeCN (50 ml) at room temperature under N₂. CuI (280 mg, 20 mol %) was added and the mixture was stirred at room temperature for 10 minutes. 1-Ethyl-6-iodo-4-oxo-1,4-dihydro-quinoline-3-carboxylic acid (3.78 g, 11 mmol) was added followed by addition of Pd(PPh₃)₂Cl₂ (257 mg, 0.37 mol). The mixture was stirred at 50° C. over night. The mixture was filtered and the filtrate was concentrated. EtOAc (100 ml) was added, the suspension was put in ultrasound bath for 10 minutes and filtered. The filtrate was washed with sat. aq. NaHCO₃ (2×100 ml) and the product was extracted to water (100 ml) at pH=3.1. Aqueous layer was washed with DCM (50 ml) at pH=4.5. The product was extracted from aqueous layer with DCM (100 ml) at pH=5.7. Into the organic layer water (50 ml) was added and pH was adjusted to 9.4. The layers were separated and the organic layer was concentrated yielding 4.39 g of crude residue. Crude residue was dissolved in EtOAc (10 ml), n-hexane (60 ml) was added and the resulting suspension was left at 4° C. for 20 minutes. The residue was filtered off and purified by column chromatography (eluent DCM:MeOH:NH₃=900:150:15). Homogenous fractions were collected and evaporated to dryness yielding the title compound (1.51 g)

### Example 89

### 4″-O-{3-[3-(3-Carboxy-1-ethyl-4-oxo-1,4-dihydro-quinoline-6-yl)-propoxy]-propyl}-azithromycin

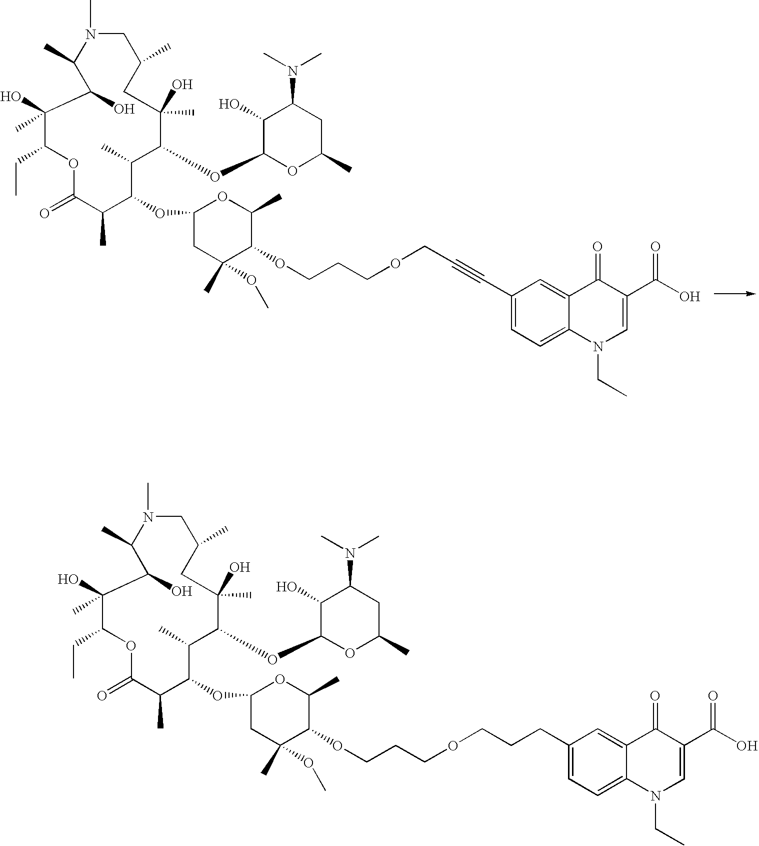

4″-O-{3-[3-(3-Carboxy-1-ethyl-4-oxo-1,4-dihydro-quinolin-6-yl)-prop-2-ynyloxy]-propyl}azithromycin (1.51 g) was dissolved in MeOH (50 ml) at room temperature. 10% Pd/C (260 mg) was added and the mixture was hydrogenated at 2 bar pressure at room temperature over night. The mixture was filtered and the filtrate was concentrated yielding 1.31 g of crude residue. The residue was gathered with additional batch of the title crude product (250 mg) and purified by column chromatography (eluent DCM:MeOH:NH₃=900:150:15) system. Homogenous fractions were collected and concentrated yielding 1.30 g. The crude residue was dissolved in EtOAc (4 ml) and the product was precipitated by addition of n-hexane (30 ml). The residue was filtered off and dried in vacuum at 45° C. for 4 hours yielding g of title compound (1.138).

### Example 90

### 4″-O-{3-[3-(3-Carboxy-1-cyclopropyl-4-oxo-1,4-dihydro-quinolin-6-yl)-prop-2-ynyloxy]-propyl}-azithromycin

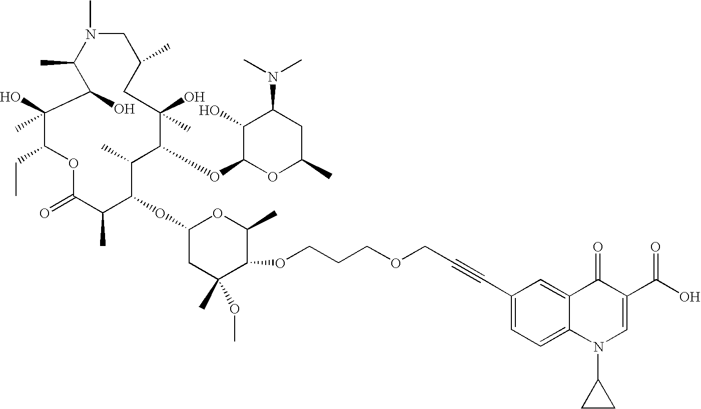

To a solution of 1-cyclopropyl-6-iodo-4-oxo-1,4-dihydro-quinoline-3-carboxylic acid (1.3 g, 3.8 mmol, 2 eq.) in MeCN (5 ml) CuI (71 mg, 0.37 mmol, 0.2 eq.) and Et₃N (2.6 ml, 19 mmol, 10 eq.) were added, the reaction was stirred at room temperature for 10 minutes under N₂ atmosphere and then heated to 50° C. Solution of 4″-O-(3-prop-2-ynyloxy-propyl)-azithromycin (1.65 g, 1.9 mmol, 3.3 g 50% pure by LC/MS) in MeCN (15 ml) and Pd(PPh₃)₂Cl₂ (66 mg, 0.09 mmol, 0.05 eq.) were added and the reaction mixture was stirred at 50° C. for 7 hours under N₂ atmosphere and at room temperature over night. EtOAc (30 ml) was added and impurities removed by filtration. Organic layer was extracted with sat. aq. NaHCO₃ (2×70 ml). To the organic layer H₂O (30 ml) was added and pH was adjusted to 3. At pH3 the product goes to the H₂O layer. Water layer was extracted with DCM (20 ml) first at pH 4.5 (according to the TLC product was still in water layer and some impurities in DCM) and then with fresh DCM (2×30 ml) at pH 5.5 (product in the DCM, pH was adjusted with NH₃/H₂O=1/1). Combined organic layers at pH 5.5 were extracted with water (20 ml) at pH 10 and evaporated in vacuum. Foamy product was precipitated from EtOAc:n-hexane yielding 1.76 g of the crude beige title product. The crude product was purified by column chromatography (eluent DCM:MeOH:NH₃=900:150:15) yielding the title compound as 1.35 g of a white foamy solid.

MS; m/z (ES): 1072.4 [MH]⁺

### Example 91

### 4″-O-{3-[3-(3-Carboxy-1-cyclopropyl-4-oxo-1,4-dihydro-quinolin-6-yl)-propoxy]-propyl}-azithromycin

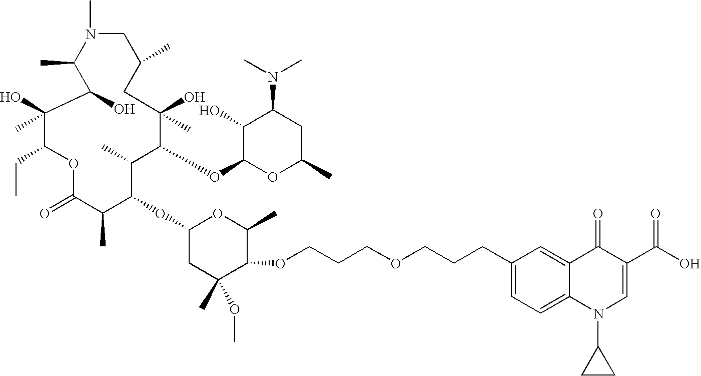

To a solution of 4″-O-{3-[3-(3-carboxy-1-cyclopropyl-4-oxo-1,4-dihydro-quinolin-6-yl)-prop-2-ynyloxy]-propyl}-azithromycin (470 mg, 0.44 mmol) in MeOH (30 ml), 10% Pd/C. (100 mg) was added and the mixture hydrogenated in a Parr apparatus at H₂-pressure of 2 bars for 18 hours. The catalyst was filtered off and the filtrate evaporated under vacuum. The product was purified by column chromatography (eluent DCM:MeOH:NH₃=90:9:1.5), precipitated from EtOAc:n-hexane and dried at 40° C. for 3 hours and at 45° C. for additional 5 hours yielding the title compound (197 mg) as a white solid.

MS; m/z (ES): 1076.4 [MH]⁺

### Example 92

### 4″-O-{2-[3-(3-Carboxy-1,4-dihydro-1-ethyl-4-oxo-6-quinolinyl)butyl]-oxyethyl}-azithromycin

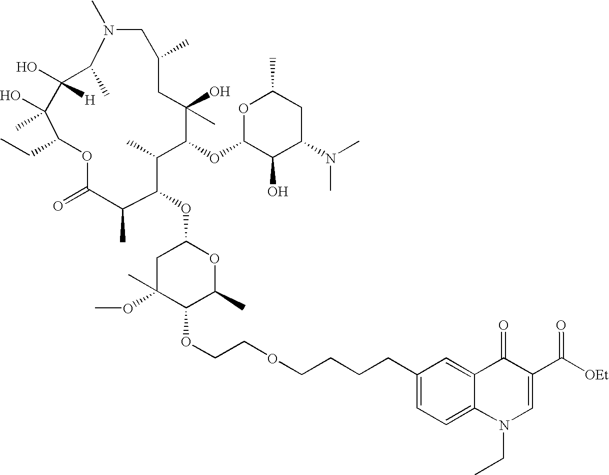

A suspension of 4″-O-(2-allyloxyethyl)-azithromycin (0.267 g, 0.32 mmol), 1-ethyl-6-allyl-4-oxo-1,4-dihydro-3-quinolinecarboxylic acid (0.092 g, 0.357 mmol) and benzylidene-bis(tricyclohexylphosphine)dichlororuthenium (60 mg) in dichloromethane (5 mL) was refluxed for 17 h. Another portion (60 mg) of benzylidene-bis(tricyclohexylphosphine)dichlororuthenium was added and the mixture refluxed for 17 h then cooled down before adding 10% Pd/C (0.15 g). After two mins the catalyst was filtered off over celite and replaced with another batch of 10% Pd/C (0.15 g) then hydrogenated at 20° C. and 1 atm for 5 h. The reaction mixture was filtered through celite, washed with methanol then concentrated. The residue was purified by preparative reverse phase HPLC (MeCN/H₂O/0.1% HCO₂H eluent) followed by chromatography on silica gel eluting with 0-10% (10:1 methanol/0.880 ammonia) in dichloromethane to give the title compound as a white solid (0.010 g); ESMS m/z 1064.8 [M+H]⁺.

### Example 93

### 4″-O-{2-[3-(3-Carboxy-1,4-dihydro-1-ethyl-4-oxo-7-quinolinyl)propyl]-oxyethyl}-azithromycin

Ethyl 1-ethyl-7-iodo-4-oxo-1,4-dihydro-quinoline-3-carboxylate was prepared from commercially available 1-ethyl-7-iodo-4-oxo-1,4-dihydro-quinoline-3-carboxylic acid in a manner analogous to the preparation of ethyl 1-ethyl-6-iodo-4-oxo-1,4-dihydro-quinoline-3-carboxylate (Intermediate 12). The reaction of ethyl 7-iodo-1-ethyl-4-oxo-1,4-dihydro-quinoline-3-carboxylate with 2′-O-acetyl-4″-O-(2-allyloxyethyl)-azithromycin 11,12-carbonate was conducted using a process analogous to that described in Example 16. Subsequent catalytic reduction of the thus formed alkene, according to the procedure described in Example 17, and removal of the protecting groups as described in Example 18, gave the title compound 4″-O-{2-[3-(3-carboxy-1,4-dihydro-1-ethyl-4-oxo-7-quinolinyl)propyl]-oxyethyl}-azithromycin. ESMS m/z 525.8 [M+2H]²⁺

### Example 94

### 4″-O-{2-[3-(3-Carboxy-1,4-dihydro-1-cyclopropyl-6-fluoro-4-oxo-7-quinolinyl)propyl]-oxyethyl}-azithromycin

Commercially available 6-fluoro-7-iodo-1-cyclopropyl-4-oxo-1,4-dihydro-quinoline-3-carboxylic acid was reacted with 4″-O-(2-allyloxyethyl)-azithromycin using a process analogous to that described in Example 58. Subsequent catalytic reduction of the alkene, according to the procedure described in Example 59, gave the title compound 4″-O-{2-[3-(3-carboxy-1,4-dihydro-1-cyclopropyl-6-fluoro-4-oxo-7-quinolinyl)propyl]-oxyethyl}-azithromycin. ESMS m/z 1080.7 [M+H]⁺

### Biological Data

Whole-cell antimicrobial activity was determined by broth microdilution using the Clinical and Laboratory Standards (CLSI) recommended procedure (Document M7-A6A7, Methods for Dilution Susceptibility Tests for Bacteria that Grow Aerobically). Compounds were dissolved in DMSO and diluted into water to produce a 640 μg/mL stock solution. The stock solution was further diluted 1:10 into Haemophilus Test Media to produce a 64 μg/mL working stock solution. A Microlab AT Plus 2 (Hamilton Co., Reno, Nev.) was used to prepare serial two-fold dilutions (50 μL aliquots) of the working stock in a 96 well microtitre plate. After the compounds were diluted, a 50 μL aliquot of the test isolate (˜1×106 cfu/ml) was added to each well of the microtitre plate. The final test concentrations ranged from 0.016-16 μg/mL. Inoculated plates were incubated at 35° C. in ambient air for 18 to 24 hours. The minimum inhibitory concentration (MIC) was determined as the lowest concentration of compound that inhibited visible growth. The compounds in the above examples gave minimum inhibitory concentrations (MICs) less than or equal to 2 microgram per millilitre against erythromycin-sensitive and erythromycin-resistant strains of Streptococcus pneumoniae and/or Streptococcus pyogenes. In a further aspect, the compounds in the above examples generally gave MICs less than 1 μg/mL against erythromycin-sensitive and erythromycin-resistant strains of Streptococcus pneumoniae and/or Streptococcus pyogenes.

However, it will appreciated by a person skilled in the art that compounds of the invention may have different levels of activity against different strains of the same bacteria.

The MICs of compounds can be determined against 42 strains of Haemophilus influenzae. The 90th percentile of these values is termed the MIC90, while the 50th percentile is termed the MIC50. The geometric mean of the 42 values may also be calculated.

### In Vivo Model of Respiratory Tract Infection (H.inf 128)

For these studies, CD-1 mice are acclimated to a restricted diet (8 grams/day) for 4 days prior to infection. Once on study, mice receive their daily ration each morning. An inoculum is obtained by placing 40 μl of Haemophilus influenzae H-128 onto Chocolate II agar plates and putting them into the CO₂ incubator for approximately 18 hours. 20 colonies are taken from the plates and put into 50 mls Mueller-Hinton broth with 5% Fildes and placed into CO₂ incubator for approximately 24 hours. The conical tube is centrifuged at 4000 rpms for 20 minutes at 37° C. The pellet is resuspended in 3 mls of Mueller Hinton broth with 5% Fildes. That tube is serially diluted 1:10 into saline to 10-7. 10-5-10-7 dilutions (200 μl each) are plated in triplicate for enumeration of the inoculum (done by scoring colony-forming units after growing overnight in CO₂ incubator). From the original conical tube, a 1:5 dilution of bacteria:melted nutrient agar is made and placed directly into 38-41° C. water bath.

For the infection, mice are anesthetized with isoflurane using an anesthesia machine. The anesthetized mice are then infected with 20 μl/mouse of bacterial suspension in molton nutrient agar by direct intra-bronchial instillation via intratrachial intubation. Mice are dosed at 1, 7, 24, 31, 48, 55, 72 and 79 hours post-infection with the corresponding compounds. Mice are sacrificed at 96 hours by CO₂ overdose and the lungs excised for enumeration of viable bacteria numbers after growing overnight in CO₂ incubator.

### Bioavailability/Oral Exposure

The bioavailability/oral exposure of compounds of the present invention may be tested using the following protocols in rat or mouse.

Rat Animal Model—Rats are surgically implanted with a Tygon catheter and are allowed a 4 day post-surgical recovery period prior to study initiation. Food (PMI 5002 certified diet) and water are provided ad-libitum until placed into a commercial auto sampling system (Culex, Bioanalytical Systems, West Lafayette, In.). Animals are acclimated to the caging and tethering system on the automated system for a minimum of 24 hours prior to study intiation. During acclimation and throughout the duration of the study, animals are given 5 food pellets/day (≈20 g) and free access to water.
Dose Preparation—Oral target doses in the rat are 50 mg/kg at 16 mL/kg and intravenous target doses are 5 mg/kg at 4 mL/kg. The intravenous dose is infused over a 1 hour period. The final dose Solution compositions are: 1.0% DMSO; 20% Encapsin (w/v) in water (po), or saline (iv) pH 3.5-4.0. Dose solution aliquots are taken (N=3, 50-μL) by weight and frozen at −80° C. until subsequent LC/MS/MS analysis along with blood/water samples.

Study regimen and sample collection—During the PO dose session, animals are orally dosed with a stainless steel gavage needle with a standard dose volume of 16 mL/kg. Rats are then allowed a 2 day washout period and crossed over for a second IV dose session in which animals are infused for 1 hour (4 mL/kg) into the femoral vein while blood samples are removed via the femoral artery catheter. When blood samples are removed from the femoral artery by the Culex instrument, samples are retained in a fraction collector at 33° F. in heparanized glass tubes. The standard timepoints at which blood samples are collected are as follows:

po: 0, 15, 30, 45, 60, 90, 120, 180, 240, 360, 480, 720, 960 and 1440 min. (75 uL per sample)
iv: 0, 20, 40, 60, 65, 75, 90, 120, 180, 240, 360, 480, 960, 1440 and 1800 min. (75 uL per sample)
Duplicate blood samples are aliquoted (25 uL) and 25 uL of water is added by a Tecan® robotic pipetting instrument. The blood/water lysates are then stored at −80° C. until subsequent quantitation by LC/MS/MS.
PK analysis—Non-compartmental pharmacokinetic analysis is performed using WinNonlin version 4.1; NCA and an internal DMPK software program, MPKR 1.74.
Mouse Animal Model—Mice are acclimated to a 2 pellets (≈8 g)/day (PMI 5002 certified diet) for a minimum of 3 days and water is provided ad-libitum. Mice are individually housed in polycarbonate shoeboxes.
Dose Preparation—Oral target doses in the mice are 300 mg/kg at 16 mL/kg. The final dose Solution compositions are: 1.0% DMSO; 20% Encapsin (w/v) in water (po), pH 3.5-5.5. Dose solution aliquots are taken (N=2-3, 50-μL) by weight and frozen at −80° C. until subsequent LC/MS/MS analysis along with blood/water samples.
Study regimen and sample collection—During the PO dose session, animals are orally dosed with a stainless steel gavage needle with a standard dose volume of 16 mL/kg. Blood samples are removed from the femoral artery via the lateral tail veins into lithium heparinized microfuge tubes. The standard timepoints at which blood samples are collected are as follows: po: 20, 40, 60, 90, 120, 180, 240, 360, 480, and 1440 (25 uL per sample). The blood is aliquoted (25 uL) and 25 uL of water is added immediately. The blood/water lysates are then stored at −80° C. until subsequent quantitation by LC/MS/MS.
PK analysis—Non-compartmental pharmacokinetic analysis is performed using WinNonlin version 4.1; NCA and an internal DMPK software program, MPKR 1.74.

Compounds of Formula (I) in which Z represents C₃₋₆alkenylene or C₃₋₆alkynylene have utility as intermediates in the preparation of compounds of Formula (I) in which Z represents C₃₋₆alkylene.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of formula (I) 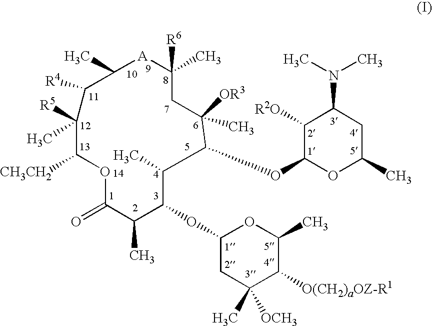
wherein:
Z represents C₃₋₆alkylene, C₃₋₆alkenylene or C₃₋₆alkynylene;
R¹ is a moiety: 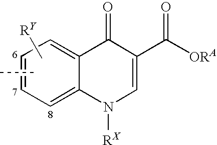
wherein
R^{A} is hydrogen, or C₁₋₆alkyl;
R^{X} represents —NR^{B}R^{C}, C₃₋₆cycloalkyl, C₁₋₆alkyl, C₃₋₆cycloalkylC₁₋₃alkyl, C₁₋₆alkoxy, —(CH₂)ₙC₁₋₄alkoxy or —(CH₂)ₙNR^{V}R^{W};
R^{Y} represents hydrogen or halogen;
R^{V} and R^{W} independently represent hydrogen or C₁₋₆alkyl;
R^{B} and R^{C} are each independently hydrogen, C₁₋₄alkyl or C₃₋₇cycloalkyl;
or a moiety 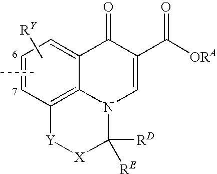
wherein:
R^{A} is hydrogen, or C₁₋₆alkyl;
R^{Y} represents hydrogen or halogen;
R^{D} and R^{E} independently represent hydrogen, or C₁₋₆alkyl such as methyl; and
Y and X independently represent O or CH₂,
with the proviso that when Y represents O, X represents CH₂ and when X represents O, Y represents CH₂;
R² is hydrogen or a hydroxyl protecting group;
R³ is hydrogen;
R⁴ is hydroxy, C₃₋₆alkenyloxy, or C₁₋₆alkoxy,
R⁵ is hydroxy, or
R⁴ and R⁵ taken together with the intervening atoms form a cyclic group: 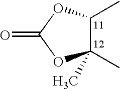
R⁶ is hydrogen;
A is a bivalent —N(R⁷)—CH₂— or —CH₂N(R⁷)—;
R⁷ is hydrogen or C₁₋₆alkyl;
a is an integer from 2 to 5
n is an integer from 2 to 4;
wherein the moiety R¹ is linked to the remainder of the molecule for the bicyclic moiety in the 6, 7 or 8 position or for the tricyclic moiety in the 6 or 7 position
and pharmaceutically acceptable derivatives thereof.

2. A compound as claimed in claim 1 wherein A is —N(R⁷)—CH₂—.

3. A compound as claimed in claim 1 wherein “a” represents 2 or 3

4. A compound as claimed in any of claim 1 wherein Z represents C₃₋₆alkylene.

5. A compound as claimed in any of claim 4 wherein Z represents n-propylene.

6. A compound as claimed in any of claim 1 wherein R^{D} represents methyl or hydrogen.

7. A compound as claimed in any of claim 1 wherein R^{E} represents methyl or hydrogen.

8. A compound as claimed in claim 1 wherein R^{X} represents —N(CH₃)₂—, methyl, ethyl, n-propyl, i-propyl, n-butyl or t-butyl, cyclopropyl, cyclopropylmethyl, methoxy, —CH₂CH₂OCH₃, —CH₂CH₂N(CH₃)₂.

9. A compound as claimed in claim 1 wherein R⁴ represents hydroxyl.

10. A compound as claimed in claim 1 wherein R⁵ represents hydroxyl.

11. A compound as claimed in claim 1 wherein R⁷ represents methyl.

12. A compound as claimed in claim 1 wherein the quinolone moiety is linked as shown below:

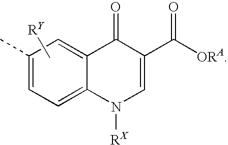

13. A compound as claimed in claim 1 wherein the tricyclic moiety is linked as shown below:

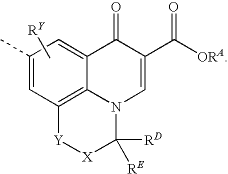

14. A compound of Formula (I) as claimed in claim 1, selected from:
4″-O-{3-[3-(3-Carboxy-1-dimethylamino-4-oxo-1,4-dihydro-quinolin-6-yl)-prop-2-en-1-yloxy]-propyl}-azithromycin;
4″-O-{3-[3-(3-Carboxy-1-dimethylamino-4-oxo-1,4-dihydro-quinolin-6-yl)-prop-2-ynyloxy]-propyl}-azithromycin;
4″-O-{3-[3-(3-Carboxy-1-dimethylamino-4-oxo-1,4-dihydro-quinolin-6-yl)-propoxy]-propyl}-azithromycin;
4″-O-{3-[3-(3-Carboxy-1-cyclopropyl-4-oxo-1,4-dihydro-quinolin-6-yl)-prop-2-en-1-yloxy]-propyl}-azithromycin;
4″-O-{3-[3-(3-Carboxy-1-ethyl-4-oxo-1,4-dihydro-quinolin-6-yl)-prop-2-en-1-yloxy]-propyl}-azithromycin;
4″-O-{3-[3-(3-Carboxy-1-methoxyeth-2-yl-4-oxo-1,4-dihydro-quinolin-6-yl)-prop-2-en-1-yloxy]-propyl}-azithromycin;
4″-O-{3-[3-(3-Carboxy-1-t-butyl-4-oxo-1,4-dihydro-quinolin-6-yl)-prop-2-en-1-yloxy]-propyl}-azithromycin;
4″-O-{3-[3-(3-Carboxy-1-i-propyl-4-oxo-1,4-dihydro-quinolin-6-yl)-prop-2-en-1-yloxy]-propyl}-azithromycin;
4″-O-{3-[3-(3-Carboxy-1-dimethylaminoeth-2-yl-4-oxo-1,4-dihydro-quinolin-6-yl)-prop-2-en-1-yloxy]-propyl}-azithromycin;
4″-O-{3-[3-(3-Carboxy-1-cyclopropyl-4-oxo-1,4-dihydro-quinolin-6-yl)-propoxy]-propyl}-azithromycin;
4″-O-{3-[3-(3-Carboxy-1-ethyl-4-oxo-1,4-dihydro-quinolin-6-yl)-propoxy]-propyl}-azithromycin;
4″-O-{3-[3-(3-Carboxy-1-methoxyeth-2-yl-4-oxo-1,4-dihydro-quinolin-6-yl)-propoxy]-propyl}-azithromycin;
4″-O-{3-[3-(3-Carboxy-1-t-butyl-4-oxo-1,4-dihydro-quinolin-6-yl)-propoxy]-propyl}-azithromycin;
4″-O-{3-[3-(3-Carboxy-1-i-propyl-4-oxo-1,4-dihydro-quinolin-6-yl)-propoxy]-propyl}-azithromycin;
4″-O-{3-[3-(3-Carboxy-1-dimethylaminoethyl-4-oxo-1,4-dihydro-quinolin-6-yl)-propoxy]-propyl}-azithromycin;
2′-O-Acetyl-4″-O-{2-[3-(3-carboxy-1,4-dihydro-1-dimethylamino-4-oxo-6-quinolinyl)prop-2-enyl]-oxyethyl}-azithromycin 11,12-carbonate ethyl ester;
2′-O-Acetyl-4″-O-{2-[3-(3-carboxy-1,4-dihydro-1-dimethylamino-4-oxo-6-quinolinyl)propyl]-oxyethyl}-azithromycin 11,12-carbonate ethyl ester;
4″-O-{2-[3-(3-Carboxy-1,4-dihydro-1-dimethylamino-4-oxo-6-quinolinyl)propyl]-oxyethyl}-azithromycin;
2′-O-Acetyl-4″-O-{2-[3-(3-carboxy-1,4-dihydro-1-ethyl-4-oxo-6-quinolinyl)prop-2-enyl]-oxyethyl}-azithromycin 11,12-carbonate ethyl ester;
2′-O-Acetyl-4″-O-{2-[3-(3-carboxy-1,4-dihydro-1-ethyl-4-oxo-6-quinolinyl)propyl]-oxyethyl}-azithromycin 11,12-carbonate ethyl ester;
4″-O-{2-[3-(3-Carboxy-1,4-dihydro-1-ethyl-4-oxo-6-quinolinyl)propyl]-oxyethyl}-azithromycin;
2′-O-Acetyl-4″-O-{5-[3-(3-carboxy-1-ethyl-4-oxo-1,4-dihydro-quinolin-6-yl)-prop-2-en-1-yloxy]-pentyl}-azithromycin-1,112-cyclic carbonate;
4″-O-{5-[3-(3-carboxy-1-ethyl-4-oxo-1,4-dihydro-quinolin-6-yl)-prop-2-en-1-yloxy]-pentyl}-azithromycin;
4″-O-{5-[3-(3-carboxy-1-ethyl-4-oxo-1,4-dihydro-quinolin-6-yl)-propyloxy]-pentyl}-azithromycin;
2′-O-Acetyl-4″-O-{2-[3-(6-carboxy-3,3-dimethyl-7-oxo-1H,7H-[1,3 oxazino]5,4,3-ij]-9-quinolinyl)prop-2-enyl]-oxyethyl}-azithromycin 11,12-carbonate ethyl ester;
2′-O-Acetyl-4″-O-{2-[3-(6-carboxy-3,3-dimethyl-7-oxo-1H,7H-[1,3 oxazino]5,4,3-ij]-9-quinolinyl)prop-1-enyl]-oxyethyl}-azithromycin 11,12-carbonate ethyl ester;
2′-O-Acetyl-4″-O-{2-[3-(6-carboxy-3,3-dimethyl-7-oxo-1H,7H-[1,3 oxazino]5,4,3-ij]-9-quinolinyl)prop-1-yl]-oxyethyl}-azithromycin 11,12-carbonate ethyl ester;
4″-O-{2-[3-(6-carboxy-3,3-dimethyl-7-oxo-1H,7H-[1,3 oxazino]5,4,3-ij]-9-quinolinyl)prop-1-yl]-oxyethyl}-azithromycin;
4″-O-{3-[4-(3-Carboxy-1-dimethylamino-4-oxo-1,4-dihydro-quinolin-6-yl)-but-3-ynyloxy]-propyl}-azithromycin;
4″-O-{3-[4-(3-Carboxy-1-ethyl-4-oxo-1,4-dihydro-quinolin-6-yl)-but-3-ynyloxy]-propyl}-azithromycin;
4″-O-{3-[5-(3-Carboxy-1-dimethylamino-4-oxo-1,4-dihydro-quinolin-6-yl)-pent-4-ynyloxy]-propyl}-azithromycin;
4″-O-{3-[5-(3-Carboxy-1-ethyl-4-oxo-1,4-dihydro-quinolin-6-yl)-pent-4-ynyloxy]-propyl}-azithromycin;
4″-O-{3-[4-(3-Carboxy-1-cyclopropyl-4-oxo-1,4-dihydro-quinolin-6-yl)-but-3-ynyloxy]-propyl}-azithromycin;
4″-O-{3-[4-(3-Carboxy-1-dimethylamino-4-oxo-1,4-dihydro-quinolin-6-yl)-butoxy]-propyl}-azithromycin;
4″-O-{3-[4-(3-Carboxy-1-ethyl-4-oxo-1,4-dihydro-quinolin-6-yl)-butoxy]-propyl}-azithromycin;
4″-O-{3-[5-(3-Carboxy-1-dimethylamino-4-oxo-1,4-dihydro-quinolin-6-yl)-pentoxy]-propyl}-azithromycin;
4″-O-{3-[5-(3-Carboxy-1-ethyl-4-oxo-1,4-dihydro-quinolin-6-yl)-pentoxy]-propyl}-azithromycin;
4″-O-{3-[4-(3-Carboxy-1-cyclopropyl-4-oxo-1,4-dihydro-quinolin-6-yl)-butoxy]-propyl}-azithromycin;
4″-O-{3-[3-(5-Carboxy-3,3-dimethyl-6-oxo-1H,6H-2-oxa-3a-aza-phenalen-8-yl)-prop-2-ynyloxy]-propyl}-azithromycin;
4″-O-{3-[3-(5-Carboxy-3,3-dimethyl-6-oxo-1H,6H-2-oxa-3a-aza-phenalen-8-yl)-propoxy]-propyl}-azithromycin;
4″-O-{3-[4-(5-Carboxy-3,3-dimethyl-6-oxo-1H,6H-2-oxa-3a-aza-phenalen-8-yl)-but-3-ynyloxy]-propyl}-azithromycin;
4″-O-{3-[4-(5-Carboxy-3,3-dimethyl-6-oxo-1H,6H-2-oxa-3a-aza-phenalen-8-yl)-butoxy]-propyl}-azithromycin;
2′-O-Acetyl-4″-O-{2-[3-(3-carboxy-1,4-dihydro-1-(2-methoxyethyl)-4-oxo-6-quinolinyl)prop-2-enyl]-oxyethyl}-azithromycin 11,12-carbonate;
2′-O-Acetyl-4″-O-{2-[3-(3-carboxy-1,4-dihydro-1-(2-methoxyethyl)-4-oxo-6-quinolinyl)prop-1-enyl]-oxyethyl}-azithromycin 11,12-carbonate;
2′-O-Acetyl-4″-O-{2-[3-(3-carboxy-1,4-dihydro-1-(2-methoxyethyl)-4-oxo-6-quinolinyl)prop-1-yl]-oxyethyl}-azithromycin 11,12-carbonate;
4″-O-{2-[3-(3-Carboxy-1,4-dihydro-1-(2-methoxyethyl)-4-oxo-6-quinolinyl)prop-1-yl]-oxyethyl}-azithromycin;
2′-O-Acetyl-4″-O-{2-[3-(3-carboxy-1,4-dihydro-1-methoxy-4-oxo-6-quinolinyl)prop-2-enyl]-oxyethyl}-azithromycin 11,12-carbonate;
2′-O-Acetyl-4″-O-{2-[3-(3-carboxy-1,4-dihydro-1-methoxy-4-oxo-6-quinolinyl)prop-1-yl]-oxyethyl}-azithromycin 11,12-carbonate;
4″-O-{2-[3-(3-Carboxy-1,4-dihydro-1-methoxy-4-oxo-6-quinolinyl)prop-1-yl]-oxyethyl}-azithromycin;
4″-O-{2-[3-(3-carboxy-1,4-dihydro-1-propyl-4-oxo-6-quinolinyl)prop-2-enyl]-oxyethyl}-azithromycin;
4″-O-{2-[3-(3-carboxy-1,4-dihydro-1-propyl-4-oxo-6-quinolinyl)propyl]-oxyethyl}-azithromycin;
4″-O-{2-[3-(3-carboxy-1,4-dihydro-1-methyl-4-oxo-6-quinolinyl)prop-2-enyl]-oxyethyl}-azithromycin;
4″-O-{2-[3-(3-carboxy-1,4-dihydro-1-methyl-4-oxo-6-quinolinyl)prop-1-enyl]-oxyethyl}-azithromycin;
4″-O-{2-[3-(3-Carboxy-1,4-dihydro-1-methyl-4-oxo-6-quinolinyl)propyl]-oxyethyl}-azithromycin;
4″-O-{2-[3-(3-Carboxy-1,4-dihydro-4-oxo-1-iso-propyl-6-quinolinyl)prop-2-enyl]-oxyethyl}-azithromycin;
4″-O-{2-[3-(3-Carboxy-1,4-dihydro-4-oxo-1-iso-propyl-6-quinolinyl)prop-1-enyl]-oxyethyl}-azithromycin;
4″-O-{2-[3-(3-Carboxy-1,4-dihydro-4-oxo-1-iso-propyl-6-quinolinyl)propyl]-oxyethyl}-azithromycin;
4″-O-{2-[3-(3-Carboxy-1,4-dihydro-4-oxo-1-cyclopropyl-6-quinolinyl)prop-2-enyl]-oxyethyl}-azithromycin;
4″-O-{2-[3-(3-Carboxy-1,4-dihydro-4-oxo-1-cyclopropyl-6-quinolinyl)prop-1-enyl]-oxyethyl}-azithromycin;
4″-O-{2-[3-(3-Carboxy-1,4-dihydro-1-cyclopropyl-4-oxo-6-quinolinyl)propyl]-oxyethyl}-azithromycin;
4″-O-{2-[3-(2-Carboxy-6,7-dihydro-1H,5H-pyrido[3,2,1-ij]-1-oxo-9-quinolinyl)prop-2-enyl]-oxyethyl}-azithromycin;
4″-O-{2-[3-(2-Carboxy-6,7-dihydro-1H,5H-pyrido[3,2,1-ij]-1-oxo-9-quinolinyl)prop-1-enyl]-oxyethyl}-azithromycin;
4″-O-{2-[3-(2-Carboxy-6,7-dihydro-1H,5H-pyrido[3,2,1-ij]-1-oxo-9-quinolinyl)propyl]-oxyethyl}-azithromycin;
4″-O-{2-[3-(6-Carboxy-7-oxo-2,3-dihydro-7H-[1,4]oxazino[2,3,4-ij]quinolin-9-yl) prop-2-enyl]-oxyethyl}-azithromycin;
4″-O-{2-[3-(6-Carboxy-7-oxo-2,3-dihydro-7H-[1,4]oxazino[2,3,4-ij]quinolin-9-yl) prop-1-enyl]-oxyethyl}-azithromycin;
4″-O-{2-[3-(6-Carboxy-7-oxo-2,3-dihydro-7H-[1,4]oxazino[2,3,4-ij]quinolin-9-yl) propyl]-oxyethyl}-azithromycin;
4″-O-{2-[3-(6-Carboxy-3,3-dimethyl-7-oxo-2,3-dihydro-7H-[1,4]oxazino[2,3,4-ij]quinolin-9-yl)prop-2-enyl]-oxyethyl}-azithromycin;
4″-O-{2-[3-(6-Carboxy-3,3-dimethyl-7-oxo-2,3-dihydro-7H-[1,4]oxazino[2,3,4-ij]quinolin-9-yl)prop-1-enyl]-oxyethyl}-azithromycin;
4″-O-{2-[3-(6-Carboxy-3,3-dimethyl-7-oxo-2,3-dihydro-7H-[1,4]oxazino[2,3,4-ij]quinolin-9-yl)propyl]-oxyethyl}-azithromycin;
4″-O-{2-[3-(3-Carboxy-1,4-dihydro-4-oxo-1-cyclo-propylmethyl-6-quinolinyl)prop-2-enyl]-oxyethyl}-azithromycin;
4″-O-{2-[3-(3-Carboxy-1,4-dihydro-4-oxo-1-cyclo-propylmethyl-6-quinolinyl)prop-1-enyl]-oxyethyl}-azithromycin;
4″-O-{2-[3-(3-Carboxy-1,4-dihydro-1-cyclo-propylmethyl-4-oxo-6-quinolinyl)propyl]-oxyethyl}-azithromycin;
4″-O-{2-[3-(3-carboxy-1,4-dihydro-1-butyl-4-oxo-6-quinolinyl)prop-2-enyl]-oxyethyl}-azithromycin ethyl ester;
4″-O-{2-[3-(3-carboxy-1,4-dihydro-1-butyl-4-oxo-6-quinolinyl)propyl]-oxyethyl}-azithromycin ethyl ester;
4″-O-{2-[3-(3-carboxy-1,4-dihydro-1-butyl-4-oxo-6-quinolinyl)propyl]-oxyethyl}-azithromycin;
2′-O-Acetyl-4″-O-{2-[3-(3-carboxy-1,4-dihydro-1-ethyl-4-oxo-6-quinolinyl)prop-1-enyl]-oxyethyl}-azithromycin 11,12-carbonate;
4″-O-{2-[3-(3-Carboxy-1,4-dihydro-1-(2-methoxyethyl)-4-oxo-6-quinolinyl)prop-1-enyl]-oxyethyl}-azithromycin;
2′-O-Acetyl-4″-O-{2-[2-(3-carboxy-1,4-dihydro-1-ethyl-4-oxo-6-quinolinyl)-1-propenyl]-oxyethyl}-azithromycin 11,12-carbonate ethyl ester;
4″-O-{2-[2-(3-carboxy-1,4-dihydro-1-ethyl-4-oxo-6-quinolinyl)-prop-1-enyl]-oxyethyl}-azithromycin;
4″-O-{3-[3-(3-Carboxy-1-ethyl-4-oxo-1,4-dihydro-quinolin-6-yl)-prop-2-ynyloxy]-propyl}-azithromycin;
4″-O-{3-[3-(3-Carboxy-1-cyclopropyl-4-oxo-1,4-dihydro-quinolin-6-yl)-prop-2-ynyloxy]-propyl}-azithromycin;
4″-O-{2-[3-(3-Carboxy-1,4-dihydro-1-ethyl-4-oxo-6-quinolinyl)butyl]-oxyethyl}-azithromycin;
4″-O-{2-[3-(3-Carboxy-1,4-dihydro-1-ethyl-4-oxo-7-quinolinyl)propyl]-oxyethyl}-azithromycin;
4″-O-{2-[3-(3-Carboxy-1,4-dihydro-1-cyclopropyl-6-fluoro-4-oxo-7-quinolinyl)propyl]-oxyethyl}-azithromycin;
or a pharmaceutically acceptable derivative thereof.

15. A compound of Formula (I), selected from:
4″-O-{4-[3-(3-carboxy-1-ethyl-4-oxo-1,4-dihydro-quinolin-6-yl)-prop-2-en-1-yloxy]-butyl}-azithromycin;
4″-O-{4-[3-(3-carboxy-1-cyclopropyl-4-oxo-1,4-dihydro-quinolin-6-yl)-prop-2-en-1-yloxy]-butyl}-azithromycin;
4″-O-{4-[3-(3-carboxy-1-dimethylamino-4-oxo-1,4-dihydro-quinolin-6-yl)-prop-2-en-1-yloxy]-butyl}-azithromycin;
4″-O-{4-[3-(3-carboxy-1-methoxyeth-2-yl-4-oxo-1,4-dihydro-quinolin-6-yl)-prop-2-en-1-yloxy]-butyl}-azithromycin;
4″-O-{4-[3-(3-carboxy-1-methoxyeth-2-yl-4-oxo-1,4-dihydro-quinolin-6-yl)-prop-2-en-1-yloxy]-butyl}-azithromycin;
4″-O-{4-[3-(3-carboxy-1-ethyl-4-oxo-1,4-dihydro-quinolin-6-yl)-propyloxy]-butyl}-azithromycin;
4″-O-{4-[3-(3-carboxy-1-cyclopropyl-4-oxo-1,4-dihydro-quinolin-6-yl)-propyloxy]-butyl}-azithromycin;
4″-O-{4-[3-(3-carboxy-1-dimethylamino-4-oxo-1,4-dihydro-quinolin-6-yl)-propyloxy]-butyl}-azithromycin;
4″-O-{4-[3-(3-carboxy-1-methoxyeth-2-yl-4-oxo-1,4-dihydro-quinolin-6-yl)-propyloxy]-butyl}-azithromycin;
or a pharmaceutically acceptable derivative thereof.

16-18. (canceled)

19. A method for the treatment of the human or non-human animal body to combat microbial infection comprising administration to a body in need of such treatment of an effective amount of a compound as claimed in claim 1, or a pharmaceutically acceptable derivative thereof.

20. A pharmaceutical composition comprising a compound as claimed in claim 1, or a pharmaceutically acceptable derivative thereof, in association with a pharmaceutically acceptable excipient, diluent and/or carrier.
